(19) United States
(12) Patent Application Publication
(10) Pub. No.: US 2026/0116958 A1
(43) Pub. Date: Apr. 30, 2026

(54) Antibodies to Botulinum Neurotoxins

(71) Applicant: The Regents of the University of California, Oakland, CA (US)

(72) Inventors: James D. Marks, San Francisco, CA (US); Jianlong Lou, San Francisco, CA (US); Yongfeng Fan, San Francisco, CA (US)

(21) Appl. No.: 18/877,574

(22) PCT Filed: Jun. 21, 2023
(86) PCT No.: PCT/US2023/068820

§ 371 Date: Dec. 20, 2024

Related U.S. Application Data
(60) Provisional application No. 63/354,551, filed on Jun. 22, 2022.

(51) Int. Cl. C07K 16/1282; A61K 39/00; A61P 31/04

(52) U.S. Cl. CPC C07K 16/1282; A61P 31/04; A61K 2039/505; C07K 2317/24; C07K 2317/76; C07K 2317/92

(57) ABSTRACT

The present disclosure provides antibodies that specifically bind to botulinum neurotoxin serotype BoNT/G. The antibodies can be used in methods to specifically bind and, in some embodiments, neutralize, botulinum neurotoxin and are therefore also useful in the treatment of pathologies associated with botulinum neurotoxin poisoning.

### CROSS-REFERENCE TO RELATED APPLICATIONS

This application claims priority to U.S. Provisional Application No. 63/354,551, filed on Jun. 22, 2022, the disclosure of which application is herein incorporated by reference in its entirety.

### STATEMENT AS TO FEDERALLY SPONSORED RESEARCH

This invention was made with government support under grant 75N93019C00057 awarded by The National Institutes of Health. The government has certain rights in the invention.

### REFERENCE TO AN ELECTRONIC SEQUENCE LISTING

The content of the electronic sequence listing titled UCSF-671WO_SEQ_LIST.xml having a size of 253,062 bytes and created on Jun. 20, 2023, is herein incorporated by reference in its entirety.

### INTRODUCTION

Botulism is caused by botulinum neurotoxin secreted by members of the genus Clostridium and is characterized by flaccid paralysis, which if not immediately fatal requires prolonged hospitalization in an intensive care unit and mechanical ventilation. Naturally occurring botulism is found in infants or adults whose gastrointestinal tracts become colonized by Clostridial bacteria (infant or intestinal botulism), after ingestion of contaminated food products (food botulism), or in anaerobic wound infections (wound botulism) (Center for Disease Control (1998) Botulism in the United States, 1899-1998. Handbook for epidemiologists, clinicians, and laboratory workers. Atlanta, Georgia U.S. Department of Health and Human Services, Public Health Service: downloadable at “bt(dot)cdc(dot)gov/agent/botulism/index(dot)asp”). Botulinum neurotoxins (BoNTs) are also classified by the Centers for Disease Control (CDC) as one of the six highest-risk threat agents for bioterrorism (the “Category A agents”), due to their extreme potency and lethality, ease of production and transport, and need for prolonged intensive care (Arnon et al. (2001) JAMA 285:1059-1070). As a result of these threats, specific pharmaceutical agents are needed for prevention and treatment of intoxication.

No specific small molecule drugs exist for prevention or treatment of botulism, but an investigational pentavalent toxoid vaccine is available from the CDC (Siegel (1988) J. Clin. Microbiol.26:2351-2356) and a recombinant vaccine is under development (Smith (1998) Toxicon 36:1539-1548). Regardless, mass civilian or military vaccination is unlikely due to the rarity of disease or exposure and the fact that vaccination would prevent subsequent medicinal use of BoNT. Toxin neutralizing antibody (Ab) can be used for pre- or post-exposure prophylaxis or for treatment (Franz et al. (1993) Pp. 473-476 In B. R. DasGupta (ed.), Botulinum and Tetanus Neurotoxins: Neurotransmission and Biomedical Aspects. Plenum Press, New York). Small quantities of both equine antitoxin and human botulinum immune globulin exist and are currently used to treat adult (Black and Gunn. (1980) Am. J. Med., 69:567-570; Hibbs et al. (1996) Clin. Infect. Dis., 23: 337-340) and infant botulism (Arnon (1993). Clinical trial of human botulism immune globulin, p. 477-482. In B. R. DasGupta (ed.), Botulinum and Tetanus Neurotoxins: Neurotransmission and Biomedical Aspects. Plenum Press, New York) respectively. Currently existing equine antitoxins include Botulism Antitoxin Heptavalent (A,B,C,D,E,F,G)—Equine (BAT), which is an FDA-Approved CDC stock for Anti-BoNT treatment following documented or suspective exposure to botulinum neurotoxin serotypes in adults and pediatric patients (Center for Biologics Evaluation and Research, Cangene Corporation, and U.S. Food and Drug Administration. “Fractionated Plasma Products—BAT (Botulism Antitoxin Heptavalent (A, B, C, D, E, F, G)—(Equine).” U.S. Food and Drug Administration Home Page. Center for Biologics Evaluation and Research, 16 Jul. 2016).

The development of monoclonal antibody (mAb) therapy for botulism is complicated by the fact that there are at least seven BoNT serotypes (A-G) (Hatheway (1995) Curr. Top. Microbio. Immunol, 195:55-75) that show little, if any, antibody cross-reactivity. While only four of the BoNT serotypes routinely cause human disease (A, B, E, and F), there has been one reported case of infant botulism caused by BoNT/C (Oguma et al. (1990) Lancet 336:1449-1450), one outbreak of foodborne botulism linked to BoNT/D (Demarchi, et al. (1958) Bull. Acad. Nat. Med., 142:580-582), and several cases of suspicious deaths where BoNT/G was isolated (Sonnabend et al. (1981) J. Infect. Dis., 143:22-27). Aerosolized BoNT/C, D, and G have also been shown to produce botulism in primates by the inhalation route (Middlebrook and Franz (1997) Botulinum Toxins, chapter 33. In F. R. Sidell, E. T. Takafuji, D. R. Franz (eds.), Medical Aspects of Chemical and Biological Warfare. TMM publications, Washington, D.C.), and would most likely also affect humans. Thus, it is likely that any one of the seven BoNT serotypes can be used as a biothreat agent.

Variability of the BoNT gene and protein sequence within serotypes has also been reported and there is evidence that such variability can affect the binding of monoclonal antibodies to BoNT/A (Kozaki et al. (1998) Infect. Immun., 66:4811-4816; Kozaki et al. (1995) Microbiol. Immunol., 39:767-774).

### SUMMARY

The present disclosure provides antibodies that specifically bind to botulinum neurotoxins. These antibodies can be used in methods to specifically bind and, in some embodiments, neutralize, botulinum neurotoxin and are therefore also useful in the treatment of botulism.

An antibody for Botulinum neurotoxin (BoNT) is provided herein may be a single chain Fv (scFv), a Fab, a F(ab)₂, an (ScFv)₂, and the like. The antibody may be an IgG. The antibody may also be in a composition comprising a pharmaceutically acceptable excipient (e.g., in a unit dosage formulation).

Methods of inhibiting and/or neutralizing the activity of BoNT in a mammal may involve administering to a mammal in need thereof a composition comprising at least one neutralizing anti-BoNT antibody as described herein. The composition may include at least two different antibodies, each of which binds to different epitopes on a BoNT subtype. The composition may include at least three, at least four, or more different antibodies, each of which may bind to different BoNT epitopes.

Compositions provided herein may comprise an antibody that specifically bind to a BoNT. The compositions typically include a first antibody that binds one or more BoNT epitopes, e.g., an antibody as described above. The compositions can optionally include a second antibody, a third antibody, or a fourth antibody, or more antibodies that bind one or more BoNT epitopes.

Nucleic acids provided herein encode one or more antibodies that are described herein. Cells containing such nucleic acids are also provided herein. Kits provided for neutralizing a BoNT may include a composition containing one or more antibodies as described herein. The kits optionally also include instructional materials teaching the use of the composition to specifically bind to a BoNT. The composition may be stored in a disposable syringe.

### BRIEF DESCRIPTION OF THE DRAWINGS

FIG. 1. Confirmation of the BoNT/G mAbs with inactive and active BoNT/G.

FIG. 2. Identification of the binding domain of the mAbs.

FIG. 3. Competition assay by flow cytometry for mAb epitope grouping.

FIG. 4. mAb clustering based on the overlap profile on BoNT/Gi.

FIG. 5. Sorting strategy and dot plots for hu6G6 affinity maturation.

FIG. 6. Sequences of colonies from the hu6G6 library sort (VH: SEQ ID Nos:225-229,13,231,13; VK: SEQ ID Nos:232,233,232; Linker: SEQ ID Nos:234,235).

FIG. 7. Affinity measurement of affinity matured Hu6G6.2 in the IgG format using KinExA. Detection used 6G10 labeled with Alexa 647.

FIG. 8. Sequences of Vk from 6G9 humanization library (SEQ ID Nos:236-245).

FIG. 9. 6G9 humanization library sorting.

FIG. 10. Sequence analysis of 6G9 variants (Top: SEQ ID Nos:246,247; Bottom: SEQ ID Nos:72,248-250,249).

FIG. 11. Measurement of KD values of 6G9 variants A2 and B9.

FIG. 12. Sorting strategy and dot plots starting with the hu6G9 error prone library.

FIG. 13. VK sequences of hu6G9 variants from error prone PCR library (Top: SEQ ID Nos:247,68; Middle: SEQ ID Nos:234,235,234; Bottom: SEQ ID Nos:251-253).

FIG. 14. Determination of KD values for hu6G9 variants.

FIG. 15. Sequences of Vk from 6G10 humanization library (SEQ ID Nos:254-264).

FIG. 16. Sorting strategy and dot plots for the hu6G10 library.

FIG. 17. Sequences of colonies from the hu6G10 library (Top: SEQ ID Nos:265,266; Bottom: SEQ ID Nos:267-271).

FIG. 18. Determination of K_{D} values for hu6G10 variants.

FIG. 19. Sorting strategy and dot plots for 6G7.1 humanization using 25 nM BoNT/Gi.

FIG. 20. Sorting hu6G7.1E random mutation library.

FIG. 21. Sequences of colonies from hu6G7.1E random mutation library (Top: SEQ ID Nos:272,46,273,274; Bottom: SEQ ID Nos:275-278).

FIG. 22. Measurement of KD values on yeast of hu6G7.1 colonies.

FIG. 23. Measurement of KD values on yeast for colonies A1 and A1ID.

FIG. 24. Affinity measurement of affinity matured variants in the IgG format using KinExA. Detection used 6G10 labeled with Alexa 647.

FIG. 25. Summary of humanization and affinity maturation strategies of four lead mAbs.

FIG. 26. Results from LD₅₀ determination for recombinant BoNT/G from Toxogen GmbH and determination of LD₅₀.

### DEFINITIONS

A “BoNT polypeptide” refers to a Botulinum neurotoxin polypeptide (e.g., a BoNT/A polypeptide, a BoNT/B polypeptide, a BoNT/C polypeptide, a BoNT/G polypeptide, and so forth). The BoNT polypeptide can refer to a full-length polypeptide or to a fragment thereof. Thus, for example, the term “BoNT/G polypeptide” refers to either a full-length BoNT/G (a neurotoxin produced by Clostridium botulinum of the type G serotype) or a fragment thereof (e.g., the heavy chain (HC) fragment).

A “BoNT serotype” refers one of the standard known BoNT serotypes (e.g., BoNT/A, BoNT/B, BoNT/C, BoNT/D, BoNT/E, BoNT/F, BoNT/G etc.).

The term “BoNT subtype” refers to botulinum neurotoxins of a particular serotype (e.g., A, B, C, D, E, F, G, etc.) that differ from each other sufficiently to produce differential antibody binding. BoNT serotypes A, B, E, and F all exist as multiple subtypes which differ from each other in amino acid sequence to an extent that impacts the ability of monoclonal antibodies (mAbs) and polyclonal serum to bind and neutralize the toxin.

A “mosaic BoNT”, as used herein, refers to a BoNT polypeptide that contains at least two contiguous amino acid sequences, each of which is derived from a different serotype or subtype.

“Derived from” in the context of an amino acid sequence or polynucleotide sequence (e.g., an amino acid sequence “derived from” BoNT/G) is meant to indicate that the polypeptide or nucleic acid has a sequence that is based on that of a reference polypeptide or nucleic acid (e.g., a naturally occurring BoNT/G or encoding nucleic acid), and is not meant to be limiting as to the source or method in which the protein or nucleic acid is made.

An “anti-BoNT antibody” refers to an antibody that binds a BoNT polypeptide, specifically binds a BoNT polypeptide with a K_{D} less than about 10⁻⁷ M, less than about 10⁻⁸ M, less than about 10⁻⁹ M, less than about 10⁻¹⁰ M, less than about 10⁻¹¹ M, or less than about 10⁻¹² M or less. In certain embodiments, “high affinity” antibodies have a K_{D} of 5 nM or less.

“Neutralization” refers to a measurable decrease in the toxicity and/or circulating level of a Botulinum neurotoxin (e.g., BoNT/G) in in vitro testing, animals, or human patient.

By “treatment” it is meant that at least an amelioration of the symptoms associated with the condition afflicting the host is achieved, where amelioration refers to at least a reduction in the magnitude of a parameter, e.g., symptom, associated with the condition being treated. As such, treatment includes situations where the condition, or at least symptoms associated therewith, are reduced or avoided. Treatment includes: (i) prevention, that is, reducing the risk of development of clinical symptoms, including causing the clinical symptoms not to develop, e.g., preventing disease progression to a harmful or otherwise undesired state; (ii) inhibition, that is, arresting the development or further development of clinical symptoms, e.g., mitigating or completely inhibiting an active disease.

“Potency” refers to the degree of protection from challenge with BoNT. This can be measured/quantified for example, as an increase in the LD₅₀ of a Botulinum neurotoxin (BoNT). In toxicology, the median lethal dose, LD₅₀ (abbreviation for “Lethal Dose, 50%”), or LCt₅₀ (Lethal Concentration & Time) of a toxic substance or radiation is the dose required to kill half the members of a tested population. The LD₅₀ usually expressed as the mass of substance administered per unit mass of test subject, such as grams of substance per kilogram of body mass. Stating it this way allows the relative toxicity of different substances to be compared, and normalizes for the variation in the size of the animals exposed (although toxicity does not always scale simply with body mass). Typically, the LD₅₀ of a substance is given in milligrams per kilogram of body weight. In the case of some toxins, the LD₅₀ may be more conveniently expressed as micrograms per kilogram (μg/kg) of body mass.

The term “high affinity” when used with respect to an antibody refers to an antibody that specifically binds to its target(s) with an affinity (K_{D}) of at least about 5 nM or less.

The following abbreviations are used herein: BoNT; Botulinum neurotoxin, BoNT/A; BoNT serotype A, BoNT/B; BoNT serotype B, BoNT/C; BoNT serotype C, BoNT/D; BoNT serotype D, BoNT/F; BoNT serotype F, BoNT/G; BoNT serotype G, Fc; fragment crystalizable, Fab′2; fragment, antigen binding, mAb; monoclonal antibody, IgG; immunoglobulin G, LD₅₀; lethal dose 50%, scFv; single chain variable fragment, VH; heavy chain variable region, Vx; kappa light chain variable region, PCR; polymerase chain reaction, AgaII or Aga2; yeast agglutinin receptor II, BoNT/A Lc; BoNT/A light chain, BoNT/B Lc; BoNT/B light chain, BoNT/B Hc; C-terminal domain of the BoNT/B heavy chain, pM; picomolar, fM; femtomolar, IU; International Unit, SD-CAA; selective dextrose casamino acids media, SG-CAA; selective galactose casamino acids media, CHO; Chinese hamster ovary cells, FACS; fluorescent activated cell sorting, K_{D}; equilibrium dissociation constant, kₒₙ; association rate constant, K_{off}; dissociation rate constant, MFI: mean fluorescent intensity.

The terms “polypeptide”, “peptide”, and “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids, which include, but are not limited to those listed in 37 CFR (§ 1.822(b)(4)). Furthermore, it should be noted that a dash at the beginning or end of an amino acid residue sequence indicates either a peptide bond to a further sequence of one or more amino acid residues or a covalent bond to a carboxyl or hydroxyl end group. However, the absence of a dash should not be taken to mean that such peptide bonds or covalent bond to a carboxyl or hydroxyl end group is not present, as it is conventional in representation of amino acid sequences to omit such.

The term “antibody” (also used interchangeably with “immunoglobulin”) encompasses polyclonal and monoclonal antibody preparations where the antibody may be of any class of interest (e.g., IgM, IgG, and subclasses thereof), as well as preparations including hybrid antibodies, altered antibodies, F(ab′)₂ fragments, F(ab) molecules, Fv fragments, scFv fragments, single chain antibodies, single domain antibodies, chimeric antibodies, humanized antibodies, and functional fragments thereof which exhibit immunological binding properties of the parent antibody molecule. The antibodies may be conjugated to other moieties, and/or may be bound to a support (e.g., a solid support), such as a polystyrene plate or bead, test strip, and the like.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region is composed of a “framework” region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, (1991) and Lefranc et al. IMGT, the international ImMunoGeneTics information System®. Nucl. Acids Res., (2005) 33:D593-D597)). A detailed discussion of the IMGTS system, including how the IMGTS system was formulated and how it compares to other systems, is provided on the World Wide Web at imgt.cines.fr/textes/IMGTScientificChart/Numbering/IMGTnumberingsTable.html. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to Kabat et al, supra, unless otherwise indicated. The three light chain CDRs, as used herein, are also referred to as “LCDR1”, “LCDR2”, and “LCDR3”. The three heavy chain variable CDRs, as used herein, are also referred to as “HCDR1”, “HCDR2”, and “HCDR3”.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies encompass intact immunoglobulins as well as a number of well characterized fragments produced by digestion with various peptidases. Thus, for example, pepsin digests an antibody below the disulfide linkages in the hinge region to produce F(ab)′₂, a dimer of Fab which itself is a light chain joined to VH-CHI by a disulfide bond. The F(ab)′, may be reduced under mild conditions to break the disulfide linkage in the hinge region thereby converting the F(ab′) 2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies, including, but are not limited to, Fab′₂, IgG, IgM, IgA, scFv, dAb, nanobodies, unibodies, and diabodies.

Antibodies and fragments of the present disclosure encompass those that are bispecific. Bispecific antibodies or fragments can be of several configurations. For example, bispecific antibodies may resemble single antibodies (or antibody fragments) but have two different antigen binding sites (variable regions). Bispecific antibodies may be produced by chemical techniques (Kranz et al. (1981) Proc. Natl. Acad. Sci., USA, 78:5807), by “polydoma” techniques (see, e.g., U.S. Pat. No. 4,474,893), or by recombinant DNA techniques. Bispecific antibodies may have binding specificities for at least two different epitopes, at least one of which is an epitope of BoNT. The BoNT binding antibodies and fragments can also be heteroantibodies. Heteroantibodies are two or more antibodies, or antibody binding fragments (e.g., Fab) linked together, each antibody or fragment having a different specificity.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three-dimensional space to form an antigen binding “surface”. This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, MD (1987).

As used herein, the terms “immunological binding” and “immunological binding properties” refer to the non-covalent interactions of the type which occur between an immunoglobulin molecule and an antigen for which the immunoglobulin is specific. The strength or affinity of immunological binding interactions can be expressed in terms of the dissociation constant (K_{D}) of the interaction, wherein a smaller Kp represents a greater affinity. Immunological binding properties of selected polypeptides can be quantified using methods well known in the art. One such method entails measuring the rates of antigen binding site/antigen complex formation and dissociation, wherein those rates depend on the concentrations of the complex partners, the affinity of the interaction, and on geometric parameters that equally influence the rate in both directions. Thus, both the “on rate constant” (kₒₙ) and the “off rate constant” (K_{off}) can be determined by calculation of the concentrations and the actual rates of association and dissociation. The ratio of k_{off}/kₒₙ enables cancellation of all parameters not related to affinity and is thus equal to the equilibrium dissociation constant K_{D} (see, generally, Davies el al. Ann. Rev. Biochem. 1990, 59:439-15 473).

An “anti-BoNT antibody” refers to an antibody that binds to a Botulinum neurotoxin(s) (e.g., BoNT/G, BoNT/C, etc.). Thus, for example the term “anti-BoNT/G antibody”, as used herein refers to an antibody that specifically binds to a BoNT/G polypeptide. An example of an antibody of the present disclosure may bind to an LC domain of a BoNT/G polypeptide.

Antibodies derived from anti-BoNT antibodies have a binding affinity of about 1.6×10⁻⁸ M or better and can be derived by screening libraries of single chain Fv fragments displayed on phage or yeast constructed from heavy (V_{H}) and light (V_{L}) chain variable region genes obtained from mammals, including mice, immunized with botulinum toxoid, toxin, or BoNT fragments. Antibodies can also be derived by screening phage or yeast display libraries in which a known BoNT-neutralizing variable heavy (V_{H}) chain is expressed in combination with a multiplicity of variable light (V_{L}) chains or conversely a known BoNT-neutralizing variable light chain is expressed in combination with a multiplicity of variable heavy (V_{H}) chains. BoNT-neutralizing antibodies also include those antibodies produced by the introduction of mutations into the variable heavy or variable light complementarity determining regions (CDR1, CDR2 or CDR3) as described herein. Finally, BoNT-neutralizing antibodies include those antibodies produced by any combination of these modification methods as applied to the BoNT-neutralizing antibodies described herein and their derivatives.

An “epitope” is a site on an antigen (e.g., BoNT) to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

A neutralizing epitope refers to the epitope specifically bound by a neutralizing antibody.

“Isolated” refers to an entity of interest that is in an environment different from that in which the compound may naturally occur. An “isolated” compound (e.g., an “isolated” antibody) is separated from all or some of the components that accompany it in nature and may be substantially enriched, e.g., may be purified so that the compound is at least about 70% pure, at least about 80% pure, at least about 90% pure, at least about 95% pure, at least about 98% pure, at least about 99%, or greater than 99% pure, or free of impurities, contaminants, and/or components other than the compound. “Isolated” also refers to the state of a compound separated from all or some of the components that accompany it during manufacture (e.g., chemical synthesis, recombinant expression, culture medium, and the like).

A single chain Fv (“scFv”) polypeptide is a covalently linked V_{H}:V_{L} heterodimer which may be expressed from a nucleic acid including V_{H}- and V_{L}-encoding sequences either joined directly or joined by a peptide-encoding linker (Huston, et al. (1988) Proc. Nat. Acad. Sci. USA, 85:5879-5883). A number of structures are available for converting the light and heavy polypeptide chains from an antibody V region into an scFv molecule which will fold into a three-dimensional structure substantially similar to the structure of an antigen-binding site. See, e.g., U.S. Pat. Nos. 5,091,513 and 5,132,405 and 4,956,778.

Recombinant design methods may be used to develop suitable chemical structures (linkers) for converting two heavy and light polypeptide chains from an antibody variable region into a scFv molecule which will fold into a three-dimensional structure that is substantially similar to native antibody structure.

Design criteria include determination of the appropriate length to span the distance between the C-terminal of one chain and the N-terminal of the other, wherein the linker is generally formed from small hydrophilic amino acid residues that do not tend to coil or form secondary structures. Such methods have been described in the art. See, e.g., U.S. Pat. Nos. 5,091,513 and 5,132,405 to Huston et al.; and U.S. Pat. No. 4,946,778 to Ladner et al.

In this regard, the first general step of linker design involves identification of plausible sites to be linked. Appropriate linkage sites on each of the V_{H} and V_{L} polypeptide domains include those which will result in the minimum loss of residues from the polypeptide domains, and which will necessitate a linker comprising a minimum number of residues consistent with the need for molecule stability. A pair of sites defines a “gap” to be linked. Linkers connecting the C-terminus of one domain to the N-terminus of the next generally comprise hydrophilic amino acids which assume an unstructured configuration in physiological solutions and may be free of residues having large side groups which might interfere with proper folding of the V_{H} and V_{L} chains. Thus, suitable linkers generally comprise polypeptide chains of alternating sets of glycine and serine residues, and may include glutamic acid and lysine residues inserted to enhance solubility. One particular linker has the amino acid sequence (Gly₄Ser)₃ (SEQ ID NO: 221). Another example of a suitable linker is a linker that has the amino acid sequence comprising 2 or 3 repeats of [(Ser)₄Gly] (SEQ ID NO: 222), such as [(Ser)₄Gly]₃ (SEQ ID NO: 223), and the like. Nucleotide sequences encoding such linker moieties can be readily provided using various oligonucleotide synthesis techniques known in the art (see, e.g., Sambrook, supra.).

The phrase “specifically binds to” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to a protein under such conditions may require an antibody that is selected for its specificity for a particular protein. For example, BoNT/G-neutralizing antibodies can be raised to BoNT/G protein(s) that specifically bind to BoNT/G protein(s), and not to other proteins present in a sample. A variety of immunoassay formats may be used to select antibodies specifically immunoreactive with a particular protein. For example, solid-phase enzyme-linked immunosorbent assay (ELISA) immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

The term “conservative substitution” is used in reference to proteins or peptides to reflect amino acid substitutions that do not substantially alter the activity (specificity or binding affinity) of the molecule. Typically, conservative amino acid substitutions involve substituting one amino acid for another amino acid with similar chemical properties (e.g., charge or hydrophobicity). The following six groups each contain amino acids that are typical conservative substitutions for one another: 1) Alanine (A), Serine(S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### DETAILED DESCRIPTION

The present disclosure provides antibodies that specifically bind to botulinum neurotoxins. The antibodies and derivatives thereof can be used in methods to specifically bind and, in some embodiments, neutralize, botulinum neurotoxin and are therefore also useful in the treatment of botulism.

Botulinum neurotoxin is produced by the anaerobic bacterium Clostridium botulinum. Botulinum neurotoxin poisoning (botulism) arises in a number of contexts including, but not limited to food poisoning (food borne botulism), infected wounds (wound botulism), “infant botulism” from ingestion of spores and production of toxin in the intestine of infants, and as a chemical/biological warfare agent. Botulism is a paralytic disease that typically begins with cranial nerve involvement and progresses caudally to involve the extremities. In acute cases, botulism can prove fatal. For each BoNT serotype, there can be multiple subtypes of BoNT. Antibodies of the present disclosure encompass antibodies that specifically bind BoNT/G.

In certain aspects, antibodies that are particularly efficient in the neutralization of a botulism neurotoxin (BoNT) serotype G are disclosed. In certain aspects, these antibodies have a high affinity for BoNT/G and each of the various antibodies is highly specific for an epitope of BoNT/G. In certain embodiments, neutralizations of BoNT may also be accomplished by using one, two, three, four, or more different antibodies directed against each of the epitopes, or by bispecific or polyspecific antibodies with specificities for two, three, or four or more BoNT epitopes.

Compositions comprising at least two, at least three, at least four of the antibodies disclosed herein are also provided. In certain embodiments, the composition may comprise two or more antibodies that bind overlapping (partial or complete overlapping) or non-overlapping epitopes on the BoNT.

Compositions provided herein may further comprise antibodies selected from those described in PCT Pub. Nos. WO 07/094754, WO 05/016232, WO 09/008916, and WO 2010/014854), each of which PCT publication is herein incorporated by reference in its entirety.

As indicated above, the antibodies provided by the present disclosure bind to botulinum neurotoxin serotype G and in some embodiments, neutralize the neurotoxin. Neutralization, in this context, refers to a measurable decrease in the toxicity and/or circulating level of the target neurotoxin. Such a decrease in toxicity can also be measured in vitro by a number of methods well known to those of skill in the art. One such assay involves measuring the time to a given percentage (e.g., 50%) twitch tension reduction in a hemidiaphragm preparation. Toxicity reduction can be determined in vivo, e.g., as an LD₅₀ in a test animal (e.g., mouse) BoNT in the presence of one or more putative neutralizing antibodies. The neutralizing antibody or antibody combination can be combined with the botulinum neurotoxin prior to administration, or the animal can be administered the antibody prior to, simultaneous with, or after administration of the neurotoxin. The rate of clearance of BoNT mediated by a test antibody, or combination of test antibodies, can be measured (e.g., in mice) by administering labeled BoNT (e.g., radiolabeled BoNT) and measuring the levels of BoNT in the serum and the liver and other organs over time in the presence or absence of test antibody or antibodies (see, e.g., Ravichandran et al. (2006) J Pharmacol Exp Ther 318:1343-1351).

As the antibodies of the present disclosure act to specifically bind to, and in some embodiments, neutralize botulinum neurotoxins, they are useful in the treatment of pathologies associated with botulinum neurotoxin poisoning. The treatments comprise administering to the poisoned subject (e.g., human or non-human mammal) a quantity of one or more neutralizing antibodies sufficient to neutralize (e.g., mitigate or eliminate) symptoms of BoNT poisoning.

Such treatments are most desired and efficacious in acute cases (e.g., where vital capacity is less than 30-40 percent of predicted and/or paralysis is progressing rapidly and/or hypoxemia with absolute or relative hypercarbia is present. These antibodies can also be used to treat early cases with symptoms milder than indicated (to prevent progression) or even prophylactically (a use the military envisions for soldiers going in harm's way). Treatment with the neutralizing antibody can be provided as an adjunct to other therapies (e.g., antibiotic treatment).

The antibodies provided by this disclosure can also be used for the rapid detection/diagnosis of botulism and thereby supplement and/or replace previous laboratory diagnostics.

This disclosure also provides the epitopes specifically bound by botulinum neurotoxin antibodies described herein.

### I. Botulinum Neurotoxin (BoNT)-Binding Antibodies

Anti-BoNT antibodies may be defined based on their affinity to one or more BoNT serotypes/subtypes. Numbering system used herein for toxins is based on Lacy et al. (1999) J. Mol. Biol. 291:1091-1104. A number of subtypes are known for each BoNT serotype. Thus, for example, BoNT/A subtypes include, but are not limited to, BoNT/A1, BoNT/A2, BoNT/A3, and the like. It is also noted, for example, that the BoNT/A1 subtype includes, but is not limited to 62A, NCTC 2916, ATCC 3502, and Hall hyper (Hall Allergan) and are identical (99.9-100% identity at the amino acid level) and have been classified as subtype A1. The BoNT/A2 sequences (Kyoto-F and FRI-A2H) (Willems, et al. (1993) Res. Microbiol. 144:547-556) are 100% identical at the amino acid level. Another BoNT/A subtype, e.g. A3, is produced by a strain called Loch Maree that killed a number of people in an outbreak in Scotland. Similarly, a number of subtypes are known for BoNT/F. These include, but are not limited to, BoNT/F1, BoNT/F2, BoNT/F3, and the like, with amino acid sequence difference between subtypes as high as 36%. The subject antibodies encompass antibodies with high affinity to one or more BoNT serotypes/subtypes (e.g., BoNT/C, BoNT/D, and/or BoNT/G).

Serotypes that can be bound by the subject antibodies include BoNT/G (UniProt: Q60393).

Moreover, without being bound to a particular theory, these antibodies can be more efficient in neutralizing Botulinum neurotoxin, particularly when used in combination with one or more different neutralizing antibodies.

The antibodies of the present disclosure can be used individually, and/or in combination with each other, and/or in combination with other known anti-BoNT antibodies (see, e.g., Application Pub. No: 20080124328, 20020155114, 20040175385, 20020155114, and PCT Pub. Nos. WO 07/094754, WO 05/016232, WO 09/008916, and WO 2010/014854, which are incorporated herein by reference for all purposes). These antibodies can be used individually, and/or in combination with each other, and/or in combination with other known anti-BoNT antibodies to form bispecific or polyspecific antibodies.

Amino acid sequences of CDRs, Framework regions, VH, and VL chains of various antibodies disclosed herein are provided in Tables 1-6. CDRs listed in Tables 1-6 are defined according to Kabat et al., Sequences of Proteins of Immunological Interest,” E. Kabat et al., U.S. Department of Health and Human Services, (1991). It will be appreciated that the amino acid sequence of a CDR can also be defined using alternative systems, which will be readily apparent to and applied by the ordinarily skilled artisan (see, e.g., Lefranc et al. IMGT, the international ImMunoGeneTics information system. Nucl. Acids Res., (2005) 33:D593-D597)). A detailed discussion of the IMGTS system, including how the IMGTS system was formulated and how it compares to other systems, is provided on the World Wide Web at imgt.cines.fr/textes/IMGTScientificChart/Numbering/IMGTnumberingsTable.html.

Using the teachings and the sequence information provided herein, the variable light and variable heavy chains can be joined directly or through a linker (e.g., (Gly₄Ser)₃, SEQ ID NO: 221) to form a single-chain Fv antibody. The various CDRs and/or framework regions can be used to form human antibodies, chimeric antibodies, antibody fragments, polyvalent antibodies, and the like.

An antibody encompassed by the present disclosure may have the binding specificity (i.e., in this context, the same CDRs, or substantially the same CDRs) of an antibody set forth in Tables 1-6. An antibody encompassed by the present disclosure may therefore include one or more CDRs as set forth in a V_{H} or V_{L} sequence shown in Tables 1-6 and, additionally, may have at least 80%, 85%, 90%, 95%, 96%, 97%, 98%, 99% identity, or up to 100% to a full-length V_{H} or V_{L} sequence shown in Tables 1-6. In certain embodiments, an antibody of the present disclosure may include a VH chain comprising the HCDRs1-3 of an antibody listed in Tables 1-6 and heavy chain framework regions 1-4, where each framework region has a sequence identity of at least 80%, 85%, 90%, 95%, 96%, 97%, 98%, 99% to the sequence of a corresponding framework region listed in Tables 1-6. In certain embodiments, an antibody of the present disclosure may include a VL chain comprising the LCDRs1-3 of an antibody listed in Tables 1-6 and light chain framework regions 1-4, where each framework region has a sequence identity of at least 80%, 85%, 90%, 95%, 96%, 97%, 98%, 99% to the sequence of a corresponding framework region listed in Tables 1-6.

Anti-BoNT antibodies of the present disclosure have a binding affinity (K_{D}) for a BoNT protein of 10⁻⁶ M or lower, of 10⁻⁷ M or lower, 10⁻⁸ M or lower, 10⁻⁹ M or lower, 10⁻¹⁰ M or lower, 10⁻¹¹ M or lower, or 10⁻¹² M or lower. Some examples of K_{D}s for BoNT/G fall in the following ranges: between about 2×10⁻¹² M to about 5×10⁻¹⁰ M, between about 5×10⁻¹⁰ to about 1×10⁻⁹ M, between 1×10⁻⁹ to 5×10⁻⁹ M, between 5×10⁻⁹ to 1×10⁻⁸ M, between 1×10⁻⁸ to 5×10⁻⁸ M, between 5×10⁻⁸ to 1×10⁻⁷ M, between 1×10⁻⁷ to 5×10⁻⁶ M.

In some cases, an antibody of the present disclosure has a K_{D} with a Botulinum neurotoxin of about 5 nM or less. In some cases, the antibody binds to BoNT/G with high affinity

An antibody encompassed by the present disclosure may have a binding affinity (K_{D}) for a BoNT/G protein between about 1×10⁻¹² M to about 1×10⁻⁶ M.

An antibody encompassed by the present disclosure may comprise a VH chain comprising HCDRs1-3 and H-FRs1-4 and a VL chain comprising LCDRs1-3 and L-FRs1-4 of a pair of VH chain and a LH chain of an antibody listed in Table 1 and may have binding affinity for BoNT/G in the range of about 4×10⁻¹¹ M to 3×10⁻⁸ M. In some embodiments, an antibody of the present disclosure may comprise a VH chain comprising HCDRs1-3 and H-FRs1-4 and a VL chain comprising LCDRs1-3 and L-FRs1-4 of the Hu6G6.2 antibody and may have a binding affinity for BoNT/G in the range of about 4×10⁻¹¹ M to 3×10⁻¹⁰ M.

An antibody encompassed by the present disclosure may comprise a VH chain comprising HCDRs1-3 and H-FRs1-4 and a VL chain comprising LCDRs1-3 and L-FRs1-4 of a pair of VH chain and a LH chain of an antibody listed in Table 2 and may have binding affinity for BoNT/G in the range of about 2×10⁻¹¹ M to 4×10⁻⁹ M. In some embodiments, an antibody of the present disclosure may comprise a VH chain comprising HCDRs1-3 and H-FRs1-4 and a VL chain comprising LCDRs1-3 and L-FRs1-4 of the Hu6G7.2 antibody and may have a binding affinity for BoNT/G in the range of about 2×10⁻⁹ M to 5×10⁻¹¹ M.

An antibody encompassed by the present disclosure may comprise a VH chain comprising HCDRs1-3 and H-FRs1-4 and a VL chain comprising LCDRs1-3 and L-FRs1-4 of a pair of VH chain and a LH chain of an antibody listed in Table 3 and may have binding affinity for BoNT/G in the range of about 2×10⁻¹¹ M to 2×10⁻⁸ M. In some embodiments, an antibody of the present disclosure may comprise a VH chain comprising HCDRs1-3 and H-FRs1-4 and a VL chain comprising LCDRs1-3 and L-FRs1-4 of the Hu6G9.1 antibody and may have a binding affinity for BoNT/G in the range of about 2×10⁻¹¹ M to 3×10⁻⁹ M.

An antibody encompassed by the present disclosure may comprise a VH chain comprising HCDRs1-3 and H-FRs1-4 and a VL chain comprising LCDRs1-3 and L-FRs1-4 of a pair of VH chain and a LH chain of an antibody listed in Table 4 and may have binding affinity for BoNT/G in the range of about 8×10⁻¹² M to 3×10⁻⁹ M. In some embodiments, an antibody of the present disclosure may comprise a VH chain comprising HCDRs1-3 and H-FRs1-4 and a VL chain comprising LCDRs1-3 and L-FRs1-4 of the Hu6G10 antibody and may have a binding affinity for BoNT/G in the range of about 8×10⁻¹² M to 6×10⁻¹⁰ M.

An antibody encompassed by the present disclosure may comprise a VH chain comprising HCDRs1-3 and H-FRs1-4 and a VL chain comprising LCDRs1-3 and L-FRs1-4 of a pair of VH chain and a LH chain of an antibody listed in Table 5 and may have binding affinity for BoNT/G in the range of about 4×10⁻¹¹ M to 2×10⁻⁷ M. In some embodiments, an antibody of the present disclosure may comprise a VH chain comprising HCDRs1-3 and H-FRs1-4 and a VL chain comprising LCDRs1-3 and L-FRs1-4 of the Hu6G11.2 antibody and may have a binding affinity for BoNT/G in the range of about 4×10⁻¹¹ M to 6×10⁻¹⁰ M.

An antibody encompassed by the present disclosure may comprise a VH chain comprising HCDRs1-3 and H-FRs1-4 and a VL chain comprising LCDRs1-3 and L-FRs1-4 of a pair of VH chain and a LH chain of an antibody listed in Table 6 and may have binding affinity for BoNT/G in the range of about 1×10⁻¹¹ to 3×10⁻⁷ M.

The antibody of the present disclosure may also be defined by the epitope or the domain of BoNT bound by the antibody. The antibodies provided here may encompass those that bind to one or more epitopes or a specific domain of a BoNT to which an antibody containing one or more of the CDRs set forth in Tables 1-6 bind. Epitopes bound by an antibody may be described by a specific BoNT domain and/or the residues therein that contribute to the interaction between the antibody and a BoNT protein. Domains bound by the certain antibodies are identified in Table 7 and in the example section.

In some cases, the antibody of the present disclosure binds to BoNT/G. In some cases, the antibody of the present disclosure binds to an epitope of BoNT/G. The 6G6 antibody and its derivatives (e.g., Hu6G6, Hu6G6.1, and Hu6G6.2) bind to an epitope that is part of the GLC domain epitope cluster. The 6G7 antibody and its derivative (e.g., 6G7.1, Hu6G7.2, and Hu6G7.1) bind to an epitope that is part of the GH_{N} domain epitope cluster. The 103D12 antibody binds to a second epitope that is part of the GH_{N} domain epitope cluster. The 6G9 antibody and its derivatives (e.g., Hu6G9.1), 101D2 antibody, and 102B3 antibody bind to a first epitope of the GH_{C} domain epitope cluster. The 6G10 antibody and its derivatives (e.g., Hu6G10), 102C12 antibody, and 103F7 antibody bind to a second epitope of the GH_{C} domain epitope cluster. The 6G11 antibody and its derivatives (e.g., Hu6G11, Hu6G11.1, Hu6G11.2, and Hu6G11.3) bind to a third epitope of the GH_{C} domain epitope cluster.

The subject antibody may also be defined by the epitope shared by one or more antibodies. The ability of a particular antibody to recognize the same and/or overlapping epitope as another antibody can be determined by the ability of one antibody to competitively inhibit binding of the second antibody to the antigen. Competitive inhibition of binding may also be referred to as cross-reactivity of antibodies. Any of a number of competitive binding assays can be used to measure competition between two antibodies to the same antigen. For example, a sandwich ELISA assay can be used for this purpose. Additional methods for assaying for cross-reactivity are described later below.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays used to assess competitive binding.

Accordingly, the present disclosure provides antibodies that bind to a Botulinum neurotoxin G. The antibodies provided by the present disclosure also encompass those that compete with antibodies of the present disclosure for binding to a BoNT/G.

In some cases, the antibody that binds to a Botulinum neurotoxin comprises a VH chain comprising a CDR1, CDR2, and CDR3 and a V_{L} chain comprising a CDR1, CDR2 and CDR3 of a pair of V_{H} chain and V_{L} chain of an antibody listed in Tables 1-6. In some cases, the V_{H} chain further comprises a FR1, FR2, FR3, and FR4 and the V_{L} chain further comprises a FR1, FR2, FR3, and FR4 of a pair of V_{H} chain and V_{L} chain of an antibody listed in Tables 1-6. In some cases, the antibody listed in Tables 1-6 is the Hu6G6.2 antibody, the Hu6G7.2 antibody, the Hu6G9.1 antibody, the Hu6G10 antibody, or the Hu6G11.2 antibody.

In some cases, the antibody comprises a VH chain comprising a CDR1, CDR2, and CDR3 of an antibody listed in Tables 1-6. In some cases, the VH chain further comprises a FRI, FR2, FR3, and FR4 of an antibody listed in Tables 1-6. In some cases, the antibody listed in Tables 1-6 is the Hu6G6.2 antibody, the Hu6G7.2 antibody, the Hu6G9.1 antibody, the Hu6G10 antibody, or the Hu6G11.2 antibody.

In some cases, the antibody comprises a V_{L} chain comprising a CDR1, CDR2, and CDR3 of an antibody listed in Tables 1-6. In some cases, the V_{L} chain further comprises a FRI, FR2, FR3, and FR4 of an antibody listed in Tables 1-6. In some cases, the antibody listed in Tables 1-6 is the Hu6G6.2 antibody, the Hu6G7.2 antibody, the Hu6G9.1 antibody, the Hu6G10 antibody, or the Hu6G11.2 antibody.

In some cases, the antibody that binds to a Botulinum neurotoxin comprises a VH chain comprising the amino acid sequence of the V_{H} chain and a V_{L} chain comprising the amino acid sequence of the V_{L} chain of an antibody listed in Tables 1-6. In some cases, the antibody comprises a V_{H} chain comprising an amino acid sequence having at least 80%, at least 90%, or at least 95% sequence identity to a V_{H} chain and a V_{L} chain comprising an amino acid sequence having at least 80%, at least 90%, or at least 95% sequence identity to a V_{L} chain of an antibody listed in Tables 1-6. In some cases, the antibody listed in Tables 1-6 is the Hu6G6.2 antibody, the Hu6G7.2 antibody, the Hu6G9.1 antibody, the Hu6G10 antibody, or the Hu6G11.2 antibody.

In some cases, the antibody that binds to a Botulinum neurotoxin (e.g., BoNT/G) comprises a V_{H} chain comprising a HCDR1 having the amino acid sequence set forth in SEQ ID NO:3, a HCDR2 having the amino acid sequence set forth in SEQ ID NO:14, and a HCDR3 having the amino acid sequence set forth in SEQ ID NO:7; and a V_{L} chain comprising a LCDR1 having the amino acid sequence set forth in SEQ ID NO:28, a LCDR2 having the amino acid sequence set forth in SEQ ID NO:30, and a LCDR3 having the amino acid sequence set forth in SEQ ID NO:32. In some cases, the antibody comprises a V_{H} chain having the amino acid sequence set forth in SEQ ID NO:16. In some cases, the antibody comprises a V_{L} chain having amino acid sequence set forth in SEQ ID NO:26. In some cases, the antibody is the Hu6G6.2 antibody.

In some cases, the antibody that binds to a Botulinum neurotoxin (e.g., BoNT/G) comprises a VH chain comprising a HCDR1 having the amino acid sequence set forth in SEQ ID NO:36, a HCDR2 having the amino acid sequence set forth in SEQ ID NO:38, and a HCDR3 having the amino acid sequence set forth in SEQ ID NO:47; and a VL chain comprising a LCDR1 having the amino acid sequence set forth in SEQ ID NO:50, a LCDR2 having the amino acid sequence set forth in SEQ ID NO:52, and a LCDR3 having the amino acid sequence set forth in SEQ ID NO:54. In some cases, the antibody comprises a V_{H} chain having the amino acid sequence set forth in SEQ ID NO:46. In some cases, the antibody comprises a V_{L} chain having amino acid sequence set forth in SEQ ID NO:56. In some cases, the antibody is the Hu6G7.2 antibody.

In some cases, the antibody that binds to a Botulinum neurotoxin (e.g., BoNT/G) comprises a VH chain comprising a HCDR1 having the amino acid sequence set forth in SEQ ID NO:63, a HCDR2 having the amino acid sequence set forth in SEQ ID NO:65, and a HCDR3 having the amino acid sequence set forth in SEQ ID NO:67; and a VL chain comprising a LCDR1 having the amino acid sequence set forth in SEQ ID NO:79, a LCDR2 having the amino acid sequence set forth in SEQ ID NO:81, and a LCDR3 having the amino acid sequence set forth in SEQ ID NO:83. In some cases, the antibody comprises a V_{H} chain having the amino acid sequence set forth in SEQ ID NO:68. In some cases, the antibody comprises a V_{L} chain having amino acid sequence set forth in SEQ ID NO:77. In some cases, the antibody is the Hu6G9.1 antibody.

In some cases, the antibody that binds to a Botulinum neurotoxin (e.g., BoNT/G) comprises a VH chain comprising a HCDR1 having the amino acid sequence set forth in SEQ ID NO:87, a HCDR2 having the amino acid sequence set forth in SEQ ID NO:89, and a HCDR3 having the amino acid sequence set forth in SEQ ID NO:91; and a V_{L} chain comprising a LCDR1 having the amino acid sequence set forth in SEQ ID NO:105, a LCDR2 having the amino acid sequence set forth in SEQ ID NO:106, and a LCDR3 having the amino acid sequence set forth in SEQ ID NO:108. In some cases, the antibody comprises a V_{H} chain having the amino acid sequence set forth in SEQ ID NO:92. In some cases, the antibody comprises a V_{L} chain having amino acid sequence set forth in SEQ ID NO:103. In some cases, the antibody is the Hu6G10 antibody.

In some cases, the antibody that binds to a Botulinum neurotoxin (e.g., BoNT/G) comprises a VH chain comprising a HCDR1 having the amino acid sequence set forth in SEQ ID NO:63, a HCDR2 having the amino acid sequence set forth in SEQ ID NO:65, and a HCDR3 having the amino acid sequence set forth in SEQ ID NO:113; and a VL chain comprising a LCDR1 having the amino acid sequence set forth in SEQ ID NO:117, a LCDR2 having the amino acid sequence set forth in SEQ ID NO:125, and a LCDR3 having the amino acid sequence set forth in SEQ ID NO:121. In some cases, the antibody comprises a V_{H} chain having the amino acid sequence set forth in SEQ ID NO:115. In some cases, the antibody comprises a V_{L} chain having amino acid sequence set forth in SEQ ID NO:124. In some cases, the antibody is the Hu6G11.2 antibody.

TABLE 1a
Amino acid sequence of VH and VL chains of anti-BoNT 6G6 antibody derivatives

| | Clone | Variable Chain Sequence |
|---|---|---|
| VH | 6G6 | EVQLQQSGAELVKPGASVKLSCTASGFNIKDTYMHWVKQRPEQGLEWIGRIDPANGNTK |
| | | YDPKFQGKATITADTSSNTAYLQLSSLTSEDTAVYYCASGPNYYGTTWFAYWGQGTTVT |
| | | VSS (SEQ ID NO: 1) |
| | IIu6G6 | QVQLVQSGAEVKKPGASVKLSCKASGFNIKDTYMHWVRQAPGQGLEWIGRIDPANGNT |
| | | KYDPKFQGRATITADTSINTAYLELSRLRSDDTA VYYCASGPNYYGTTWFA YWGQGTTV |
| | | TVSS (SEQ ID NO: 9) |
| | Hu6G6.1 | QVQLVQSGAEVKKPGASVKLSCKASGFNIKDTYMHWVRQAPGQGLEWIGRIDPENGNT |
| | | KYDPKFQGRATITAYKSINTAYLELSRLRSDDTAVYYCASGPNYYGTTWFAYWGQGTTV |
| | | TVSS (SEQ ID NO: 13) |
| | Hu6G6.2 | QVQLVQSGAEVKKPGASVKLSCKASGFNIKDTYMHWVRQAPGQGLEWIGRIDPENGNT |
| | | KYDPKFQGRATITAYKSINRAYLELSRLRSDDTAVYYCASGPNYYGTTWFAYWGQGTT |
| | | VTVSS (SEQ ID NO: 16) |
| VI | 6G6 | QIVLTQSPAILSASPGEKVTMTCRASSSVSYMHWYQQKPGSSPRLWIYRTSNLASGVPAR |
| | | FSGSGSGTSYSLTISSMEAEDAATYYCQQYHSYPRTFGGGTKLEIKR (SEQ ID NO: 18) |
| | Hu6G6 | EIVLTQSPSTLSASVGDRVTITCRASHGISNYLAWYQQKPGRAPKLLIYKASTLHIGVPSRF |
| | | SGSGSGTEFALTISSLQPDDESTYYCQQYNSYLPHTFGQGTRLEIKR (SEQ ID NO: 26) |
| | Hu6G6.1 | EIVLTQSPSTLSASVGDRVTITCRASHGISNYLAWYQQKPGRAPKLLIYKASTLHIGVPSRF |
| | | SGSGSGTEFALTISSLQPDDFSTYYCQQYNSYLPHTFGQGTRLEIKR (SEQ ID NO: 26) |
| | Hu6G6.2 | EIVLTQSPSTLSASVGDRVTITCRASHGISNYLAWYQQKPGRAPKLLIYKASTLHIGVPSRF |
| | | SGSGSGTEFALTISSLQPDDESTYYCQQYNSYLPHTFGQGTRLEIKR (SEQ ID NO: 26) |

TABLE 1b
Amino acid sequence of VH and VL CDR and Framework regions of anti-BONT 6G6
derivative antibodies

| | Clone | Framework1 | CDR1 | Framework2 | CDR2 | Framework3 | CDR3 | Framework4 |
|---|---|---|---|---|---|---|---|---|
| VH | 6G6 | EVQLQQSGA | DTYMH | WVKQRPEQ | RIDPANG | KATITADTSS | GPNYY | WGQGT |
| | | ELVKPGASV | (SEQ ID | GLEWIG | NTKYDPK | NTAYLQLSS | GTTWF | TVTVSS |
| | | KLSCTASGEN | NO: 3) | (SEQ ID NO: | FQG (SEQ | LTSEDTAVY | AY | (SEQ ID |
| | | IK (SEQ ID | | 4) | ID NO: 5) | YCAS (SEQ | (SEQ ID | NO: 8) |
| | | NO: 2) | | | | ID NO: 6) | NO: 7) | |
| | Hu6G6 | QVQLVQSGA | DTYMH | WVRQAPGQ | RIDPANG | RATITADTSI | GPNYY | WGQGT |
| | | EVKKPGASV | (SEQ ID | GLEWIG | NTKYDPK | NTAYLELSR | GTTWF | TVTVSS |
| | | KLSCKASGF | NO: 3) | (SEQ ID NO: | FQG (SEQ | LRSDDTAVY | AY | (SEQ ID |
| | | NIK (SEQ ID | | 11) | ID NO: 5) | YCAS (SEQ | (SEQ ID | NO: 8) |
| | | NO: 10) | | | | ID NO: 12) | NO: 7) | |
| | Hu6G6.1 | QVQLVQSGA | DTYMH | WVRQAPGQ | RIDPENG | RATITAYKSI | GPNYY | WGQGT |
| | | EVKKPGASV | (SEQ ID | GLEWIG | NTKYDPK | NTAYLELSR | GTTWF | TVTVSS |
| | | KLSCKASGF | NO: 3) | (SEQ ID NO: | FQG (SEQ | LRSDDTAVY | AY | (SEQ ID |
| | | NIK (SEQ ID | | 11) | ID NO: 14) | YCAS (SEQ | (SEQ ID | NO: 8) |
| | | NO: 10) | | | | ID NO: 15) | NO: 7) | |
| | Hu6G6.2 | QVQLVQSGA | DTYMH | WVRQAPGQ | RIDPENG | RATITAYKSI | GPNYY | WGQGT |
| | | EVKKPGASV | (SEQ ID | GLEWIG | NTKYDPK | NRAYLELSR | GTTWF | TVTVSS |
| | | KLSCKASGF | NO: 3) | (SEQ ID NO: | FQG (SEQ | LRSDDTAVY | AY | (SEQ ID |
| | | NIK (SEQ ID | | 11) | ID NO: 14) | YCAS (SEQ | (SEQ ID | NO: 8) |
| | | NO: 10) | | | | ID NO: 17) | NO: 7) | |
| VL | 6G6 | QIVLTQSPAI | RASSSV | WYQQKPGS | RTSNLAS | GVPARFSGS | QQYHS | FGGGT |
| | | LSASPGEKVT | SYMH | SPRLWIY | (SEQ ID | GSGTSYSLTI | YPRT | KLEIKR |
| | | MTC (SEQ ID | (SEQ ID | (SEQ ID NO: | NO: 22) | SSMEAEDAA | (SEQ ID | (SEQ ID |
| | | NO: 19) | NO: 20) | 21) | | TYYC (SEQ | NO: 24) | NO: 25) |
| | | | | | | ID NO: 23) | | |
| | Hu6G6 | EIVLTQSPST | RASHGI | WYQQKPGR | KASTLHI | GVPSRFSGSG | QQYNS | FGQGT |
| | | LSASVGDRV | SNYLA | APKLLIY | (SEQ ID | SGTEFALTIS | YLPHT | RLEIKR |
| | | TITC (SEQ ID | (SEQ ID | (SEQ ID NO: | NO: 30) | SLQPDDFSTY | (SEQ ID | (SEQ ID |
| | | NO: 27) | NO: 28) | 29) | | YC (SEQ ID | NO: 32) | NO: 33) |
| | | | | | | NO: 31) | | |
| | Hu6G6.1 | EIVLTQSPST | RASHGI | WYQQKPGR | KASTLHI | GVPSRFSGSG | QQYNS | FGQGT |
| | | LSASVGDRV | SNYLA | APKLLIY | (SEQ ID | SGTEFALTIS | YLPHT | RLEIKR |
| | | TITC (SEQ ID | (SEQ ID | (SEQ ID NO: | NO: 30) | SLQPDDFSTY | (SEQ ID | (SEQ ID |
| | | NO: 27) | NO: 28) | 29) | | YC (SEQ ID | NO: 32) | NO: 33) |
| | | | | | | NO: 31) | | |
| | Hu6G6.2 | EIVLTQSPST | RASHGI | WYQQKPGR | KASTLHI | GVPSRFSGSG | QQYNS | FGQGT |
| | | LSASVGDRV | SNYLA | APKLLIY | (SEQ ID | SGTEFALTIS | YLPHT | RLEIKR |
| | | TITC (SEQ ID | (SEQ ID | (SEQ ID NO: | NO: 30) | SLQPDDFSTY | (SEQ ID | (SEQ ID |
| | | NO: 27) | NO: 28) | 29) | | YC (SEQ ID | NO: 32) | NO: 33) |
| | | | | | | NO: 31) | | |

TABLE 2a
Amino acid sequence of VH and VL chains of anti-BoNT 6G7 derivative antibodies

| | Clone | Variable Chain Sequence |
|---|---|---|
| VH | 6G7.1 | DVQLQESGPELVKPGASVKVSCKASDYSFTDYNIYWVKQSHGKSLEWIGYIDPYNGGTS |
| | | YNQKFKGKATLTVDKSSSTAFMYLNSLTSEDSAVYYCALYGYDPDYIDYWGQGTTLTV |
| | | SS (SEQ ID NO: 34) |
| | Hu6G7.1 | QVQLVQSGAEVKKPGASVKVSCKASDYSFTDYNIYWVRQTPGQGLEWIGYIDPYNGGT |
| | | SYNQKFKGRATLTVDKSISTAYMELSRLRSDDTAVYYCALYGYDPDYIDYWGQGTTLT |
| | | VSS (SEQ ID NO: 42) |
| | Hu6G7.2 | QVQLVQSGAEVKKPGASVKVSCKASDYSFTDYNIYWVRQTPGQGLEWIGYIDPYNGGT |
| | | SYNQKFKGRATLTVDKSISTAYMELSRLRSDDTAVYYCALYGYDPDYTEYWGQGTTLT |
| | | VSS (SEQ ID NO: 46) |
| VL | 6G7.1 | QIVLTQSPAIMSASPGEKVTITCSASSSVSYLHWFQQKPGTSPKL WIYSTSNLASGVPARFS |
| | | GSGSGTSYSLTISRMEAEDAATYYCQQRSSYPYTFGGGTKLEIKA (SEQ ID NO: 48) |
| | Hu6G7.1 | DIVLTQSPASMAVSPGQRVTITCSASSSVSYLHWFQQKPGQSPKLWIYSTSNLASGVPARF |
| | | SGSGSGTDYTLTINPMEAEDTATYYCQQRSSYPYTFGQGTKLEIKR (SEQ ID NO: 56) |
| | Hu6G7.2 | DIVLTQSPASMAVSPGQRVTITCSASSSVSYLHWFQQKPGQSPKLWIYSTSNLASGVPARF |
| | | SGSGSGTDYTLTINPMEAEDTATYYCQQRSSYPYTFGQGTKLEIKR (SEQ ID NO: 56) |

TABLE 2b
Amino acid sequence of VH and VL CDR and Framework regions of anti-BONT 6G7
derivative antibodies

| | Clone | Framework1 | CDR1 | Framework2 | CDR2 | Framework3 | CDR3 | Framework4 |
|---|---|---|---|---|---|---|---|---|
| VH | 6G7.1 | DVQLQESGPE | DYNIY | WVKQSHG | YIDPYNG | KATLTVDKSS | YGYDP | WGQGTTLT |
| | | LVKPGASVK | (SEQ ID | KSLEWIG | GTSYNQ | STAFMYLNSL | DYIDY | VSS (SEQ ID |
| | | VSCKASDYSF | NO: 36) | (SEQ ID NO: | KFKG | TSEDSAVYYC | (SEQ ID | NO: 41) |
| | | T (SEQ ID NO: | | 37) | (SEQ ID | AL (SEQ ID | NO: 40) | |
| | | 35) | | | NO: 38) | NO: 39) | | |
| | Hu6G7.1 | QVQLVQSGA | DYNIY | WVRQTPGQ | YIDPYNG | RATLTVDKSIS | YGYDP | WGQGTTLT |
| | | EVKKPGASV | (SEQ ID | GLEWIG | GTSYNQ | TAYMELSRLR | DYIDY | VSS (SEQ ID |
| | | KVSCKASDY | NO: 36) | (SEQ ID NO: | KFKG | SDDTAVYYCA | (SEQ ID | NO: 41) |
| | | SFT (SEQ ID | | 44) | (SEQ ID | L (SEQ ID NO: | NO: 40) | |
| | | NO: 43) | | | NO: 38) | 45) | | |
| | Hu6G7.2 | QVQLVQSGA | DYNIY | WVRQTPGQ | YIDPYNG | RATLTVDKSIS | YGYDP | WGQGTTLT |
| | | EVKKPGASV | (SEQ ID | GLEWIG | GTSYNQ | TAYMELSRLR | DYTEY | VSS (SEQ ID |
| | | KVSCKASDY | NO: 36) | (SEQ ID NO: | KFKG | SDDTAVYYCA | (SEQ ID | NO: 41) |
| | | SFT (SEQ ID | | 44) | (SEQ ID | L (SEQ ID NO: | NO: 47) | |
| | | NO: 43) | | | NO: 38) | 45) | | |
| VL | 6G7.1 | QIVLTQSPAI | SASSSV | WFQQKPGT | STSNLAS | GVPARFSGSG | QQRSS | FGGGTKLEI |
| | | MSASPGEKV | SYLH | SPKLWIY | (SEQ ID | SGTSYSLTISR | YPYT | KA (SEQ ID |
| | | TITC (SEQ ID | (SEQ ID | (SEQ ID NO: | NO: 52) | MEAEDAATY | (SEQ ID | NO: 55) |
| | | NO: 49) | NO: 50) | 51) | | YC (SEQ ID | NO: 54) | |
| | | | | | | NO: 53) | | |
| | Hu6G7.1 | DIVLTQSPAS | SASSSV | WFQQKPGQ | STSNLAS | GVPARFSGSG | QQRSS | FGQGTKLEI |
| | | MAVSPGQRV | SYLH | SPKLWIY | (SEQ ID | SGTDYTLTINP | YPYT | |
| | | TITC (SEQ ID | (SEQ ID | (SEQ ID NO: | NO: 52) | MEAEDTATYY | (SEQ ID | KR (SEQ ID |
| | | NO: 57) | NO: 50) | 58) | | C (SEQ ID NO: | NO: 54) | NO: 60) |
| | | | | | | 59) | | |
| | Hu6G7.2 | DIVLTQSPAS | SASSSV | WFQQKPGQ | STSNLAS | GVPARFSGSG | QQRSS | FGQGTKLEI |
| | | MAVSPGQRV | SYLH | SPKLWIY | (SEQ ID | SGTDYTLTINP | YPYT | |
| | | TITC (SEQ ID | (SEQ ID | (SEQ ID NO: | NO: 52) | MEAEDTATYY | (SEQ ID | KR (SEQ ID |
| | | NO: 57) | NO: 50) | 58) | | C (SEQ ID NO: | NO: 54) | NO: 60) |
| | | | | | | 59) | | |

TABLE 3a
Amino acid sequence of VH and VL chains of anti-BONT 6G9 derivative antibodies

| | Clone | Variable Chain Sequence |
|---|---|---|
| VH | 6G9 | EVQLQQSGDDLVKPGASVKMSCKASGYTFTSYWMHWVKQRPGQGLEWIGYINPSTGYTEYN |
| | | QKFKDKATLTADKSSSTAYMQLSSLTSEDSA VYYCARGGNYVDYWGQGTTLTVSS (SEQ ID |
| | | NO: 61) |
| | Hu6 | QVQLVQSGAEVKKPGASVKMSCKASGYTFTSYWMHWVRQAPGQGLEWVGYINPSTGYTEY |
| | G9.1 | NQKFKDRATLTADKSTSTAYMELRSLRSDDTAVYYCARGGNYVDYWGQGTTLTVSS (SEQ |
| | | ID NO: 68) |
| VL | 6G9 | EIVLTQSPAIMSASPGEKVTISCSASSSVSYMYWYQQKPGSSPKPWIYRTSNLASGVPARFSGS |
| | | GSGTSYSLTISSMEAEDAATYYCQQWSSNPYTFGGGTKLEIKR (SEQ ID NO: 72) |
| | Hu6 | DIQMTQSPSTLSASVGERVTITCRASHGISSWLA WYQQKPGKAPKRLIYAVSTLQTGVPSRFSD |
| | G9.1 | SGSGTDLTLTINSLQPEDFATYYCQDLNSYPLSFGGGTKVEIKR (SEQ ID NO: 77) |

TABLE 3b
Amino acid sequence of VH and VL CDR and Framework regions of anti-BONT 6G9
derivative antibodies

| | Clone | Framework1 | CDR1 | Framework2 | CDR2 | Framework3 | CDR3 | Framework4 |
|---|---|---|---|---|---|---|---|---|
| VH | 6G9 | EVQLQQSGD | SYWMH | WVKQRPGQ | YINPSTGY | KATLTADKS | GGNY | WGQGTTLT |
| | | DLVKPGASV | (SEQ ID | GLEWIG | TEYNQKF | SSTAYMQLS | VDY | VSS (SEQ ID |
| | | KMSCKASGY | NO: 63) | (SEQ ID NO: | KD (SEQ | SLTSEDSAV | (SEQ | NO: 41) |
| | | TFT (SEQ ID | | 64) | ID NO: 65) | YYCAR (SEQ | ID NO: | |
| | | NO: 62) | | | | ID NO: 66) | 67) | |
| | Hu6 | QVQLVQSGA | SYWMH | WVRQAPGQ | YINPSTGY | RATLTADKS | GGNY | WGQGTTLT |
| | G9.1 | EVKKPGASV | (SEQ ID | GLEWVG | TEYNQKF | TSTAYMELR | VDY | VSS (SEQ ID |
| | | KMSCKASGY | NO: 63) | (SEQ ID NO: | KD (SEQ | SLRSDDTAV | (SEQ | NO: 41) |
| | | TFT (SEQ ID | | 70) | ID NO: 65) | YYCAR (SEQ | ID NO: | |
| | | NO: 69) | | | | ID NO: 71) | 67) | |
| VL | 6G9 | EIVLTQSPAIM | SASSSV | WYQQKPGS | RTSNLAS | GVPARFSGS | QQWS | FGGGTKLEI |
| | | SASPGEKVTIS | SYMY | SPKPWIY | (SEQ ID | GSGTSYSLTI | SNPYT | KR (SEQ ID |
| | | C (SEQ ID NO: | (SEQ ID | (SEQ ID NO: | NO: 22) | SSMEAEDAA | (SEQ | NO: 25) |
| | | 73) | NO: 74) | 75) | | TYYC (SEQ | ID NO: | |
| | | | | | | ID NO: 23) | 76) | |
| | Hu6 | DIQMTQSPST | RASHGI | WYQQKPGK | AVSTLQT | GVPSRFSDS | QDLNS | FGGGTKVEI |
| | G9.1 | LSASVGERVT | SSWLA | APKRLIY | (SEQ ID | GSGTDLTLTI | YPLS | KR (SEQ ID |
| | | ITC (SEQ ID | (SEQ ID | (SEQ ID NO: | NO: 81) | NSLOPEDFA | (SEQ | NO: 84) |
| | | NO: 78) | NO: 79) | 80) | | TYYC (SEQ | ID NO: | |
| | | | | | | ID NO: 82) | 83) | |

TABLE 4a
Amino acid sequence of VH and VL chains of anti-BoNT 6G10 derivative antibodies

| | Clone | Variable Chain Sequence |
|---|---|---|
| VH | 6G10 | EVQLQQSGPELVKPGASVKISCKASGYSFTGYFMHWVKQSHVKSLEWIGRINPYNGDIIYN |
| | | QNFKDKASLTVDKSSSTAYMELHSLTSEDSAVYYCASLFGLYYFDYWGQGTTLTVSS (SEQ |
| | | ID NO: 85) |
| | Hu6G10 | QVQLVQSGAEVKKPGATVKISCKASGYSFTGYFMHWVRQAPGKGLEWIGRINPYNGDIIY |
| | | NQNFKDRATLTVDKSTSTAYMELSSLRSEDTAVYYCASLFGLYYFDYWGQGTTLTVSS |
| | | (SEQ ID NO: 92) |
| VL | 6G10 | EIVLTQSPAIMSASPGEKVTMTCSASSSVSYMHWYQQKSGTSPKRWIYDTSKLASGVPARF |
| | | SGSGSGTSYSLTISSMEAEDAATYYCQQWSSNPLTFGAGTKLELKR (SEQ ID NO: 96) |
| | Hu6G10 | DVVMTQSPSSVSASVGDRVTITCRASQRISNHLNWYQQKPGKAPKRLIYAASSLESGVPSRF |
| | | SGSGSGTEFTLTISSLQPEDFATYFCLQHNSYPYTFGQGTKVEIKR (SEQ ID NO: 103) |

TABLE 4b
Amino acid sequence of VH and VL CDR and Framework regions of anti-BONT 6G10
derivative antibodies

| | Clone | Framework1 | CDR1 | Framework2 | CDR2 | Framework3 | CDR3 | Framework4 |
|---|---|---|---|---|---|---|---|---|
| VH | 6G10 | EVQLQQSGP | GYFMH | WVKQSHVK | RINPYN | KASLTVDKSS | LFGLY | WGQGTTLT |
| | | ELVKPGASV | (SEQ ID | SLEWIG | GDIIYNQ | STAYMELHS | YFDY | VSS (SEQ ID |
| | | KISCKASGY | NO: 87) | (SEQ ID NO: | NFKD | LTSEDSAVYY | (SEQ ID | NO: 41) |
| | | SFT (SEQ ID | | 88) | (SEQ ID | CAS (SEQ ID | NO: 91) | |
| | | NO: 86) | | | NO: 89) | NO: 90) | | |
| | Hu6G10 | QVQLVQSG | GYFMH | WVRQAPGK | RINPYN | RATLTVDKST | LFGLY | WGQGTTLT |
| | | AEVKKPGAT | (SEQ ID | GLEWIG | GDIIYNQ | STAYMELSSL | YFDY | VSS (SEQ ID |
| | | VKISCKASG | NO: 87) | (SEQ ID NO: | NFKD | RSEDTAVYY | (SEQ ID | NO: 41) |
| | | YSFT (SEQ | | 94) | (SEQ ID | CAS (SEQ ID | NO: 91) | |
| | | ID NO: 93) | | | NO: 89) | NO: 95) | | |
| VL | 6G10 | EIVLTQSPAI | SASSSV | WYQQKSGT | DTSKLA | GVPARFSGSG | QQWSS | FGAGTKLE |
| | | MSASPGEKV | SYMH | SPKRWIY | S (SEQ ID | SGTSYSLTISS | NPLT | |
| | | TMTC (SEQ | (SEQ ID | (SEQ ID NO: | NO: 100) | MEAEDAATY | (SEQ ID | LKR (SEQ |
| | | ID NO: 97) | NO: 98) | 99) | | YC (SEQ ID | NO: 101) | ID NO: 102) |
| | | | | | | NO: 23) | | |
| | Hu6G10 | DVVMTQSPS | RASQRI | WYQQKPGK | AASSLES | GVPSRFSGSG | LOHNS | FGQGTKVE |
| | | SVSASVGDR | SNHLN | APKRLIY | (SEQ ID | SGTEFTLTISS | YPYT | IKR (SEQ ID |
| | | VTITC (SEQ | (SEQ ID | (SEQ ID NO: | NO: 106) | LQPEDFATYF | (SEQ ID | NO: 109) |
| | | ID NO: 104) | NO: 105) | 80) | | C (SEQ ID NO: | NO: 108) | |
| | | | | | | 107) | | |

TABLE 5a
Amino acid sequence of VH and VL chains of anti-BONT 6G11 derivative antibodies

| | Clone | Variable Chain Sequence |
|---|---|---|
| VH | Hu6G11 | QVQLVQSGAEVAKPGASVKMSCKASGYTFTSYWMHWVRQAPGQGLEWIGYINPSTGY |
| | | TEYNQKFKDRATLTADKSTSTAYMELSSLRSEDTAVYYCARGYFSFAYWGQGTLVTVS |
| | | S (SEQ ID NO: 110) |
| | Hu6G11.1 | QVQLVQSGAEVAKPGASVKMSCKASGYTFTSYWMHWVRQAPGQGLEWIGYINPSTGY |
| | | TEYNQKFKDRATLTADKSKSTAYMELSSLRSEDTAVYYCARGYFSFAYWGQGTLVTVS |
| | | S (SEQ ID NO: 115) |
| | Hu6G11.2 | QVQLVQSGAEVAKPGASVKMSCKASGYTFTSYWMHWVRQAPGQGLEWIGYINPSTGY |
| | . | TEYNQKFKDRATLTADKSKSTAYMELSSLRSEDTA VYYCARGYFSFAYWGQGTLVTVS |
| | | S (SEQ ID NO: 115) |
| | Hu6G11.3 | QVQLVQSGAEVAKPGASVKMSCKASGYTFTSYWMHWVRQAPGQGLEWIGYINPSTGY |
| | | TEYNQKFKDRATLTADKSTSTAYMELSSLRSEDTAVYYCARGYFSFAYWGQGTLVTVS |
| | | S (SEQ ID NO: 110) |
| VL | Hu6G11 | DIVMTQSPSSLSASVRDRVTITCQASHDIKNFLNWFQQKPGKAPKLLVYDASNLQVGVP |
| | | SRFSASGYGTEFTLTISSLQPEDFATYYCQQANSFPRTFGQGTKVEIKR (SEQ ID NO: 115) |
| | Hu6G11.1 | DIVMTQSPSSLSASVRDRVTITCQASHDIKNFLNWFQQKPGKAPKLLVYDASYLQVGVP |
| | | SRFSASGYGTEFTLTISSLQPEDFATYYCQQANSFPRTFGQGTKVEIKR (SEQ ID NO: 122) |
| | Hu6G11.2 | DIVMTQSPSSLSASVRDRVTITCQASHDIKNFLNWFQQKPGKAPKLLVYDASHLQVGVP |
| | . | SRFSASGYGTEFTLTISSLQPEDFATYYCQQANSFPRTFGQGTKVEIKR (SEQ ID NO: 124) |
| | Hu6G11.3 | EIVLTQSPSTLSASIGDRVTITCRASEGIYHWLAWYQQKPGKAPKLLIYKASSLASGAPSR |
| | | FSGSGSGTDFSLTISSLQPEDSATYFCQQAHSFPPTFGGGTKVDIKR (SEQ ID NO: 126) |

TABLE 5b
Amino acid sequence of VH and VL CDR and Framework regions of anti-BONT 6G11
derivative antibodies

| | Clone | Framework1 | CDR1 | Framework3 | CDR2 | Framework3 | CDR3 | Framework4 |
|---|---|---|---|---|---|---|---|---|
| VH | Hu6G11 | QVQLVQS | SYWM | WVRQAPG | YINPST | RATLTADKST | GYFSF | WGQGTLV |
| | | GAEVAKP | H (SEQ | QGLEWIG | GYTEY | STAYMELSSL | AY | TVSS (SEQ |
| | | GASVKMS | ID NO: | (SEQ ID | NQKFK | RSEDTAVYYC | (SEQ ID | ID NO: 114) |
| | | CKASGYTF | 63) | NO: 11) | D (SEQ | AR (SEQ ID | NO: | |
| | | T (SEQ ID | | | ID NO: | NO: 112) | 113) | |
| | | NO: 111) | | | 65) | | | |
| | Hu6G11.1 | QVQLVQS | SYWM | WVRQAPG | YINPST | RATLTADKSK | GYFSF | WGQGTLV |
| | | GAEVAKP | H (SEQ | QGLEWIG | GYTEY | STAYMELSSL | AY | TVSS (SEQ |
| | | GASVKMS | ID NO: | (SEQ ID | NQKFK | RSEDTAVYYC | (SEQ ID | ID NO: 114) |
| | | CKASGYTF | 63) | NO: 11) | D (SEQ | AR (SEQ ID | NO: | |
| | | T (SEQ ID | | | ID NO: | NO: 116) | 113) | |
| | | NO: 111) | | | 65) | | | |
| | Hu6G11.2 | QVQLVQS | SYWM | WVRQAPG | YINPST | RATLTADKSK | GYFSF | WGQGTLV |
| | | GAEVAKP | H (SEQ | QGLEWIG | GYTEY | STAYMELSSL | AY | TVSS (SEQ |
| | | GASVKMS | ID NO: | (SEQ ID | NQKFK | RSEDTAVYYC | (SEQ ID | ID NO: 114) |
| | | CKASGYTF | 63) | NO: 11) | D (SEQ | AR (SEQ ID | NO: | |
| | | T (SEQ ID | | | ID NO: | NO: 116) | 113) | |
| | | NO: 111) | | | 65) | | | |
| | Hu6G11.3 | QVQLVQS | SYWM | WVRQAPG | YINPST | RATLTADKST | GYFSF | WGQGTLV |
| | | GAEVAKP | H (SEQ | QGLEWIG | GYTEY | STAYMELSSL | AY | TVSS (SEQ |
| | | GASVKMS | ID NO: | (SEQ ID | NQKFK | RSEDTAVYYC | (SEQ ID | ID NO: 114) |
| | | CKASGYTF | 63) | NO: 11) | D (SEQ | AR (SEQ ID | NO: | |
| | | T (SEQ ID | | | ID NO: | NO: 112) | 113) | |
| | | NO: 111) | | | 65) | | | |
| VL | Hu6G11 | DIVMTQSP | QASHD | WFQQKPG | DASNL | GVPSRFSASG | QQANS | FGQGTKVE |
| | | SSLSASVR | IKNFLN | KAPKLLVY | QV | YGTEFTLTISS | FPRT | IKR (SEQ |
| | | DRVTITC | (SEQ ID | (SEQ ID | (SEQ ID | LQPEDFATYY | (SEQ ID | ID NO: 109) |
| | | (SEQ ID | NO: | NO: 118) | NO: | C (SEQ ID NO: | NO: | |
| | | NO: 116) | 117) | | 119) | 120) | 121) | |
| | Hu6G11.1 | DIVMTQSP | QASHD | WFQQKPG | DASYL | GVPSRFSASG | QQANS | FGQGTKVE |
| | | SSLSASVR | IKNFLN | KAPKLLVY | QV | YGTEFTLTISS | FPRT | IKR (SEQ |
| | | DRVTITC | (SEQ ID | (SEQ ID | (SEQ ID | LQPEDFATYY | (SEQ ID | ID NO: 109) |
| | | (SEQ ID | NO: | NO: 118) | NO: | C (SEQ ID NO: | NO: | |
| | | NO: 116) | 117) | | 123) | 120) | 121) | |
| | Hu6G11.2 | DIVMTQSP | QASHD | WFQQKPG | DASHL | GVPSRFSASG | QQANS | FGQGTKVE |
| | | SSLSASVR | IKNFLN | KAPKLLVY | QV | YGTEFTLTISS | FPRT | IKR (SEQ |
| | | DRVTITC | (SEQ ID | (SEQ ID | (SEQ ID | LQPEDFATYY | (SEQ ID | ID NO: 109) |
| | | (SEQ ID | NO: | NO: 118) | NO: | C (SEQ ID NO: | NO: | |
| | | NO: 116) | 117) | | 125) | 120) | 121) | |
| | Hu6G11.3 | EIVLTQSPS | RASEGI | WYQQKPG | KASSL | GAPSRFSGSG | QQAHS | FGGGTKV |
| | | TLSASIGD | YHWLA | KAPKLLIY | AS | SGTDFSLTISS | FPPT | DIKR (SEQ |
| | | RVTITC | (SEQ ID | (SEQ ID | (SEQ ID | LQPEDSATYF | (SEQ ID | ID NO: 133) |
| | | (SEQ ID | NO: | NO: 129) | NO: | C (SEQ ID NO: | NO: | |
| | | NO: 127) | 128) | | 130) | 131) | 132) | |

TABLE 6a
Amino acid sequence of VH and VL chains of anti-BoNT/G antibodies

| | Clone | Variable Chain Sequence |
|---|---|---|
| VH | 100G7 | QVQLKQSGAELVKPGASVKLSCTASGFNIKDTYMHWVKQRPEQGLEWIARIDPANGNTK |
| | | YDPKFQGKATMTADTSSNTAYLQLSSLTSEDIAVYYCVRESPYGLYTLDYWGQGTSVTVS |
| | | S (SEQ ID NO: 134) |
| | 101D2 | QIQLQQSGAELVKPGASVKLSCKASGYTFTSSWMHWAKQRPGQGLEWIGEIHPNSGNTN |
| | | YNEKFKGKATLTVDTSSSTAYVDLSSLTSEDSAVYYCARDWDYWYFDVWGAGTSVTVS |
| | | S (SEQ ID NO: 140) |
| | 102B3 | QIQLLQSGAELAKPGASVKMSCKASGYTFTSYWMHWVKQRPGQGLEWIGYINPSTGYTE |
| | | YNQKFKDKATLTADKSSSTAYMQLSSLTSEDSAVYYCARGGYVYFDYWGQGTTLTVSS |
| | | (SEQ ID NO: 148) |
| | 102C12 | QVQLKQSGSELVRPGASVKLSCKASGYTFTSYWINWIKQRPGQGLEWIGRIAPGSGINYY |
| | | NEMFKGKATLTVDTSSSTAYIQLSSLSSEDSAVYFCAGFITKATGAMDYWGQGTSVTVSS |
| | | (SEQ ID NO: 150) |
| | 103D12 | EVKLVESGAELVKPGASVKISCKASGYSFTGYYMHWVKQSHGKSLEWIGRVNPNNGGTT |
| | | YNQKFKGKAILTVDKSSNTAYMELRSLTSEDSAVYYCAKNWGPYGSSYFDYWGQGTTLT |
| | | VSS (SEQ ID NO: 157) |
| | 103F7 | EVQLQQSGPELVKPGASVKISCKASGYSFTGYFMNWVKQSHGKSLEWIGRINPYNGDTV |
| | | YNQKFKDKATLTADKSSTTAHMELRSLASEDSAVYFCARWDGNAWFGYWGQGTLVTVS |
| | | A (SEQ ID NO: 163) |
| | 103F10 | EVQLQQSGAELAKPGASVKMSCKASGYTFTSYWMHWVKQRPGQGLEWIGYINPSTGYT |
| | | EYNQKFKDRATLTADKSSSTAYMQLTSLTSEDSAVYYCARGYFSFAYWGQGTLVTVSA |
| | | (SEQ ID NO: 169) |
| | 108bD9 | EVQLQQSGAELVRPGTSVTLSCKASGYTESDYEMHWVKQTPVHGLEWIGAIDPETGATA |
| | | YNQKFKGEATLTADKSSNTAYMELRSLTSEDSAVYYFTRCGYRYDVGEGTSFDYWGQGT |
| | | SVTVSA (SEQ ID NO: 172) |
| VL | 100G7 | DIVMSQSPAIMSASPGEKVTMTCSASSSVSYMYWYQQKPGSSPRLLIYDTSNLASGVPVRF |
| | | SGSGSGTSYSLTISRMEAEDAATYYCQQWSSYPLTFGGGTKLEIKA (SEQ ID NO: 180) |
| | 101D2 | EIVLTQSPAILSASPGEKVTMTCRARSSVSYIHWYQQKPGSSPKPWIYATSNLASGVPDRES |
| | | GSGSGTSYSLTISRVEAEDAATYYCQQWSSTPPTFGAGTKLEIKA (SEQ ID NO: 186) |
| | 102B3 | DIVMSQSPASLA VSLGQRATISCRASGNVNSYGNSFMHWYQQKPGQPPKLLIYRASNLES |
| | | GIPARFSGSGSRTDFTLTINPVEADDVATYYCQQSNEDPYTFGGGTKLEIKA (SEQ ID NO: |
| | | 193) |
| | 102C12 | DIVMSQSPTSLAVSLGQRATISCRASESVDSYGNSFMHWYQQKPGQPPKLLIYLASNLESG |
| | | VPARFSGSGSRTDFTLTIDPVEADDAATYYCQQSNEDPYTFGGGTKLEIKA (SEQ ID NO: |
| | | 200) |
| | 103D12 | DIVLTQSPASLAVSLGQRATISCRASESVDSYGNSFMHWYQQKPGQPPKLLIYLASNLESG |
| | | VPARFSGSGSRTDFTLTIDPVEADDAATYYCQQNNEDPRTFGGGTKLEINA (SEQ ID NO: |
| | | 205) |
| | 103F7 | DIVLTQSPAIMSASLGER VTMTCTASSSVSSSYLHWYQQKPGSSPKLWIYSTSNLASGVPA |
| | | RFSGSGSGTSYSLTISSVEAEDAATYYCQQYSGYPYTFGGGTKLEIKA (SEQ ID NO: 209) |
| | 103F10 | DIMMTQSPAILSASPGEKVTMTCSASSSVSYMYWYQQKPGSSPRLLIYDTSELASGVPARF |
| | | SGSGSGTSYSLTISSMEAEDAATYYCQQWSSNPYTFGGGTKLEIKA (SEQ ID NO: 215) |
| | 108bD9 | DIVLTQSPAIMSASPGEKVTITCSASSSVSYMHWPQQKPGTSPKLWIYSTSNLASGVPARIS |
| | | GSGSGTSYSLTISSVEAEDAATYYCHQRSSYPWTFGGGTKLEIKA (SEQ ID NO: 218) |

TABLE 6b
Amino acid sequence of VH and VL CDR and Framework regions
of anti-BoNT/G antibodies

| | Clone | Framework1 | CDR1 | Framework2 | CDR2 | Framework3 | CDR3 | Framework4 |
|---|---|---|---|---|---|---|---|---|
| VH | 100G7 | QVQLKQSG | DTYMH | WVKQRPEQ | RIDPAN | KATMTADTS | ESPYGL | WGQGTSVT |
| | | AEL VKPGA | (SEQ ID | GLEWIA | GNTKYD | SNTAYLQLSS | YTLDY | VSS (SEQ ID |
| | | SVKLSCTAS | NO: 3) | (SEQ ID NO: | PKFQG | LTSEDIAVYY | (SEQ ID | NO: 139) |
| | | GFNIK (SEQ | | 136) | (SEQ ID | CVR (SEQ ID | NO: 138) | |
| | | ID NO: 135) | | | NO: 5) | NO: 137) | | |
| | 101D2 | QIQLQQSG | SSWMH | WAKQRPG | EIHPNSG | KATLTVDTSS | DWDYW | WGAGTSVT |
| | | AEL VKPGA | (SEQ ID | QGLEWIG | NTNYNE | STAYVDLSSL | YFDV | VSS (SEQ ID |
| | | SVKLSCKA | NO: 142) | (SEQ ID NO: | KFKG | TSEDSAVYY | (SEQ ID | NO: 147) |
| | | SGYTFT | | 143) | (SEQ ID | CAR (SEQ ID | NO: 146) | |
| | | (SEQ ID NO: | | | NO: 144) | NO: 145) | | |
| | | 141) | | | | | | |
| | 102B3 | QIQLLQSGA | SYWMH | WVKQRPG | YINPSTG | KATLTADKSS | GGYVYF | WGQGTTLT |
| | | ELAKPGAS | (SEQ ID | QGLEWIG | YTEYNQ | STAYMQLSSL | DY (SEQ | VSS (SEQ ID |
| | | VKMSCKAS | NO: 63) | (SEQ ID NO: | KFKD | TSEDSAVYY | ID NO: | NO: 41) |
| | | GYTFT (SEQ | | 64) | (SEQ ID | CAR (SEQ ID | 149) | |
| | | ID NO: 149) | | | NO: 65) | NO: 66) | | |
| | 102C12 | QVQLKQSG | SYWIN | WIKQRPGQ | RIAPGSG | KATLTVDTSS | FITKATG | WGQGTSVT |
| | | SELVRPGAS | (SEQ ID | GLEWIG | INYYNE | STAYIQLSSLS | AMDY | VSS (SEQ ID |
| | | VKLSCKAS | NO: 152) | (SEQ ID NO: | MFKG | SEDSAVYFCA | (SEQ ID | NO: 139) |
| | | GYTFT (SEQ | | 153) | (SEQ ID | G (SEQ ID NO: | NO: 156) | |
| | | ID NO: 151) | | | NO: 154) | 155) | | |
| | 103D12 | EVKLVESG | GYYMH | WVKQSHG | RVNPNN | KAILTVDKSS | NWGPY | WGQGTTLT |
| | | AEL VKPGA | (SEQ ID | KSLEWIG | GGTTYN | NTAYMELRS | GSSYFD | VSS (SEQ ID |
| | | SVKISCKAS | NO: 159) | (SEQ ID NO: | QKFKG | LTSEDSAVYY | Y (SEQ | NO: 41) |
| | | GYSFT (SEQ | | 37) | (SEQ ID | CAK (SEQ ID | ID NO: | |
| | | ID NO: 158) | | | NO: 160) | NO: 161) | 162) | |
| | 103F7 | EVQLQQSG | GYFMN | WVKQSHG | RINPYN | KATLTADKSS | WDGNA | WGQGTLVT |
| | | PELVKPGAS | (SEQ ID | KSLEWIG | GDTVYN | TTAHMELRS | WFGY | VSA (SEQ |
| | | VKISCKASG | NO: 164) | (SEQ ID NO | QKFKD | LASEDSAVYF | (SEQ ID | ID NO: 168) |
| | | YSFT (SEQ | | 37) | (SEQ ID | CAR (SEQ ID | NO: 167) | |
| | | ID NO: 86) | | | NO: 165) | NO: 166) | | |
| | 103F10 | EVQLQQSG | SYWMH | WVKQRPG | YINPSTG | RATLTADKSS | GYFSFA | WGQGTLVT |
| | | AELAKPGA | (SEQ ID | QGLEWIG | YTEYNQ | STAYMQLTS | Y (SEQ | VSA (SEQ |
| | | SVKMSCKA | NO: 63) | (SEQ ID NO: | KFKD | LTSEDSAVYY | ID NO: | ID NO: 168) |
| | | SGYTFT | | 64) | (SEQ ID | CAR (SEQ ID | 113) | |
| | | (SEQ ID NO: | | | NO: 65) | NO: 171) | | |
| | | 170) | | | | | | |
| | 108bD9 | EVQLQQSG | DYEMH | WVKQTPV | AIDPETG | EATLTADKSS | CGYRYD | WGQGTSVT |
| | | AEL VRPGTS | (SEQ ID | HGLEWIG | ATAYNQ | NTAYMELRS | VGEGTS | VSA (SEQ |
| | | VTLSCKAS | NO: 174) | (SEQ ID NO: | KFKG | LTSEDSAVYY | FDY | ID NO: 179) |
| | | GYTFS (SEQ | | 175) | (SEQ ID | FTR (SEQ ID | (SEQ ID | |
| | | ID NO: 173) | | | NO: 176) | NO: 177) | NO: 178) | |
| VL | 100G7 | DIVMSQSPA | SASSSV | WYQQKPGS | DTSNLA | GVPVRFSGSG | QQWSSY | FGGGTKLEI |
| | | IMSASPGEK | SYMY | SPRLLIY | S (SEQ | SGTSYSLTISR | PLT (SEQ | KA (SEQ ID |
| | | VTMTC | (SEQ ID | (SEQ ID NO: | ID NO: | MEAEDAATY | ID NO: | NO: 55) |
| | | (SEQ ID NO: | NO: 74) | 182) | 183) | YC (SEQ ID | 185) | |
| | | 181) | | | | NO: 184) | | |
| | 101D2 | EIVLTQSPAI | RARSS | WYQQKPGS | ATSNLA | GVPDRFSGSG | QQWSST | FGAGTKLEI |
| | | LSASPGEKV | VSYIH | SPKPWIY | S (SEQ | SGTSYSLTISR | PPT (SEQ | KA (SEQ ID |
| | | TMTC (SEQ | (SEQ ID | (SEQ ID NO: | ID NO: | VEAEDAATY | ID NO: | NO: 192) |
| | | ID NO: 187) | NO: 188) | 75) | 189) | YC (SEQ ID | 191) | |
| | | | | | | NO: 190) | | |
| | 102B3 | DIVMSQSPA | RASGN | WYQQKPG | RASNLE | GIPARFSGSG | QQSNED | FGGGTKLEI |
| | | SLAVSLGQ | VNSYG | QPPKLLIY | S (SEQ | SRTDFTLTINP | PYT | KA (SEQ ID |
| | | RATISC | NSFMH | (SEQ ID NO: | ID NO: | VEADDVATY | (SEQ ID | NO: 55) |
| | | (SEQ ID NO: | (SEQ ID | 196) | 197) | YC (SEQ ID | NO: 199) | |
| | | 194) | NO: 195) | | | NO: 198) | | |
| | 102C12 | DIVMSQSPT | RASESV | WYQQKPG | LASNLE | GVPARFSGSG | QQSNED | FGGGTKLEI |
| | | SLAVSLGQ | DSYGN | QPPKLLIY | S (SEQ | SRTDFTLTIDP | PYT | KA (SEQ ID |
| | | RATISC | SFMH | (SEQ ID NO: | ID NO: | VEADDAATY | (SEQ ID | NO: 55) |
| | | (SEQ ID NO: | (SEQ ID | 196) | 203) | YC (SEQ ID | NO: 199) | |
| | | 201) | NO: 202) | | | NO: 204) | | |
| | 103D12 | DIVLTQSPA | RASESV | WYQQKPG | LASNLE | GVPARFSGSG | QQNNED | FGGGTKLEI |
| | | SLAVSLGQ | DSYGN | QPPKLLIY | S (SEQ | SRTDFTLTIDP | PRT (SEQ | NA (SEQ ID |
| | | RATISC | SFMH | (SEQ ID NO: | ID NO: | VEADDAATY | ID NO: | NO: 208) |
| | | (SEQ ID NO: | (SEQ ID | 196) | 203) | YC (SEQ ID | 207) | |
| | | 206) | NO: 202) | | | NO: 204) | | |
| | 103F7 | DIVLTQSPA | TASSSV | WYQQKPGS | STSNLA | GVPARFSGSG | QQYSGY | FGGGTKLEI |
| | | IMSASLGER | SSSYLH | SPKLWIY | S (SEQ | SGTSYSLTISS | PYT | KA (SEQ ID |
| | | VTMTC | (SEQ ID | (SEQ ID NO: | ID NO: | VEAEDAATY | (SEQ ID | NO: 55) |
| | | (SEQ ID NO: | NO: 211) | 212) | 52) | YC (SEQ ID | NO: 214) | |
| | | 210) | | | | NO: 213) | | |
| | 103F10 | DIMMTQSP | SASSSV | WYQQKPGS | DTSELA | GVPARFSGSG | QQWSSN | FGGGTKLEI |
| | | AILSASPGE | SYMY | SPRLLIY | S (SEQ | SGTSYSLTISS | PYT | KA (SEQ ID |
| | | KVTMTC | (SEQ ID | (SEQ ID NO: | ID NO: | MEAEDAATY | (SEQ ID | NO: 55) |
| | | (SEQ ID NO: | NO: 74) | 182) | 217) | YC (SEQ ID | NO: 76) | |
| | | 216) | | | | NO: 23) | | |
| | 108bD9 | DIVLTQSPA | SASSSV | WFQQKPGT | STSNLA | GVPARFSGSG | HQRSSY | FGGGTKLEI |
| | | IMSASPGEK | SYMH | SPKLWIY | S (SEQ | SGTSYSLTISS | PWT | KA (SEQ ID |
| | | VTITC (SEQ | (SEQ ID | (SEQ ID NO: | ID NO: | VEAEDAATY | (SEQ ID | NO: 55) |
| | | ID NO: 219) | NO: 98) | 51) | 52) | YC (SEQ ID | NO: 220) | |
| | | | | | | NO: 213) | | |

TABLE 7
Summary of properties of anti-BoNT/G antibodies

| | | | Binding | Epitope | KD scFv | |
|---|---|---|---|---|---|---|
| mAbs | | VHCDR3 | Domain | Group | on yeast | IgG K_{D} |
| 6G6 | hu6G6 | GPNYYGTTWFA | GLC | I | ~23 nM | 8.37 nM |
| | hu6G6.1 | Y (SEQ ID NO: 7) | | | 3.4 nM | 0.47 nM |
| | Hu6G6.2 | | | | 0.22 nM | 42.2 pM |
| 6G7 | 6G7.1 | YGYDPDYIDY | HN(GLCHN) | III | 0.27 nM | 26.6 pM |
| | | (SEQ ID NO: 40) | | | | |
| | Hu6G7.2 | YGYDPDYTEY | | | 1.42 nM | 51.01 pM |
| | | (SEQ ID NO: 47) | | | | |
| | Hu6G7.1 | YGYDPDYIDY | | | 3.20 nM | 1.19 nM |
| | | (SEQ ID NO: 40) | | | | |
| 6G9 | 103B3 (6G9) | GGNYVDY (SEQ | GHc | V | 18.8 nM | 1.59 nM |
| | Hu6G9.1 | ID NO: 67) | | | 2.04 nM | 27.48 PM |
| 6G10 | 103B6 (6G10) | LFGLYYFDY | GHc | VI | 2.48 nM | 0.33 nM |
| | Hu6G10 | (SEQ ID NO: 91) | | | 0.52 nM | 8.36 pM |
| 6G11 | 103F10 | GYFSFAY (SEQ | GHc | VII | 104.9 nM | Not determined |
| | (6G11) | | | | | |
| | hu6G11 | ID NO: 113) | | | 4.5 nM | 531 pM |
| | Hu6G11.1 | | | | 0.32 nM | 100.7 pM |
| | Hu6G11.2 | | | | 0.56 nM | 48 PM |
| | Hu6G11.3 | | | | 0.32 nM | Not determined |

K_{D} values of scFv determined using FACS, K_{D} values of IgG determined using KinExA

### II. Potency of Botulinum Neurotoxin (BoNT)-Binding Antibodies

Without being bound to a particular theory, it is believed that the current antitoxins used to treat botulism (horse and human) have a potency of about 5000 mouse LD₅₀s/mg (human) and 55,000 mouse LD₅₀s/mg (horse).

Based on calculation, a commercially desirable antitoxin may generally have a potency greater than about 10,000 to 100,000 LD₅₀s/mg. Combinations of the antibodies described herein (e.g., two antibodies, or three antibodies) can meet this potency. Thus, this disclosure provides a combination of antibodies (e.g., two or three antibodies) that specifically bind to BoNT, and in some embodiments neutralize BoNT, at a potency of at least about 10,000 mouse LD₅₀s/mg of antibody, at least about 15,000 mouse LD₅₀s/mg of antibody, or at least about 20,000 mouse LD₅₀s/mg of antibody, at least about 25,000 mouse LD₅₀s/mg of antibody.

### III. Preparation of Anti-BoNT Antibodies

### Recombinant Expression of Anti-BoNT Antibodies

Using the information provided herein, the botulinum neurotoxin binding antibodies of the present disclosure are prepared using standard techniques well known to those of skill in the art.

For example, the polypeptide sequences provided herein (see, e.g., Tables 1-6) can be used to determine appropriate nucleic acid sequences encoding the anti-BoNT antibodies and the nucleic acids sequences then used to express one or more BoNT-neutralizing antibodies. The nucleic acid sequence(s) can be optimized to reflect particular codon “preferences” for various expression systems according to standard methods well known to those of skill in the art.

Using the sequence information provided, the nucleic acids may be synthesized according to a number of standard methods known to those of skill in the art. Oligonucleotide synthesis can be carried out on commercially available solid phase oligonucleotide synthesis machines (Needham-VanDevanter et al. (1984) Nucleic Acids Res. 12:6159-6168) or manually synthesized using, for example, the solid phase phosphoramidite triester method described by Beaucage et al. (1981) Tetrahedron Letts. 22(20): 1859-1862.

Once a nucleic acid encoding an anti-BoNT antibody is synthesized it can be amplified and/or cloned according to standard methods. Molecular cloning techniques to achieve these ends are known in the art. A wide variety of cloning and in vitro amplification methods suitable for the construction of recombinant nucleic acids are known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, CA (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel). Methods of producing recombinant immunoglobulins are also known in the art. See, Cabilly, U.S. Pat. No. 4,816,567; and Queen et al. (1989) Proc. Nat'l Acad. Sci. USA 86:10029-10033.

Examples of techniques sufficient to direct persons of skill through in vitro amplification methods, including the polymerase chain reaction (PCR), the ligase chain reaction (LCR), QB-replicase amplification and other RNA polymerase mediated techniques are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, CA (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; and Barringer et al. (1990) Gene 89, 117. Improved methods of cloning in vitro amplified nucleic acids are described in Wallace et al., U.S. Pat. No. 5,426,039.

Once the nucleic acid for an anti-BoNT antibody is isolated and cloned, one can express the gene in a variety of recombinantly engineered cells known to those of skill in the art. Examples of such cells include bacteria, yeast, filamentous fungi, insect (especially employing baculoviral vectors), plant, and mammalian cells. It is expected that those of skill in the art are knowledgeable in the numerous expression systems available for expression of antibodies.

In brief summary, the expression of natural or synthetic nucleic acids encoding anti-BoNT antibodies will typically be achieved by operably linking a nucleic acid encoding the antibody to a promoter (which is either constitutive or inducible), and incorporating the construct into an expression vector. The vectors can be suitable for replication and integration in prokaryotes, eukaryotes, or both. Typical cloning vectors contain transcription and translation terminators, initiation sequences, and promoters useful for regulation of the expression of the nucleic acid encoding the anti-BoNT antibody. The vectors optionally comprise generic expression cassettes containing at least one independent terminator sequence, sequences permitting replication of the cassette in both eukaryotes and prokaryotes, i.e., shuttle vectors, and selection markers for both prokaryotic and eukaryotic systems. See Sambrook et al (1989) supra.

To obtain high levels of expression of a cloned nucleic acid it is common to construct expression plasmids which typically contain a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky (1984) J. Bacteriol., 158:1018-1024, and the leftward promoter of phage lambda (P_{L}) as described by Herskowitz and Hagen (1980) Ann. Rev. Genet., 14:399-445 and the L-arabinose (araBAD) operon (Better (1999) Gene Exp Systems pp 95-107 Academic Press, Inc., San Diego, CA). The inclusion of selection markers in DNA vectors transformed in E. coli is also useful. Examples of such markers include genes specifying resistance to ampicillin, tetracycline, or chloramphenicol. See Sambrook et al (1989) supra for details concerning selection markers, e.g., for use in E. coli.

Expression systems for expressing anti-BoNT antibodies are available using, for example, E. coli, Bacillus sp. (see, e.g., Palva, et al. (1983) Gene 22:229-235; Mosbach et al. (1983) Nature, 302:543-545), and Salmonella. E. coli systems may also be used.

The anti-BoNT antibodies produced by prokaryotic cells may require exposure to chaotropic agents for proper folding. During purification from, e.g., E. coli, the expressed protein is optionally denatured and then renatured. This can be accomplished, e.g., by solubilizing the bacterially produced antibodies in a chaotropic agent such as guanidine HCl. The antibody is then renatured, either by slow dialysis or by gel filtration (see, e.g., U.S. Pat. No. 4,511,503). Alternatively, nucleic acid encoding the anti-BoNT antibodies may be operably linked to a secretion signal sequence such as pelB so that the anti-BoNT antibodies are secreted into the medium in correctly-folded form (Better et al (1988) Science 240:1041-1043).

Methods of transfecting and expressing genes in mammalian cells are known in the art (see e.g., Birch and Racher Adv. Drug Deliv. Rev. 2006, 58:671-685). Transducing cells with nucleic acids can involve, for example, incubating viral vectors containing anti-BoNT nucleic acids with cells within the host range of the vector (see. e.g., Goeddel (1990) Methods in Enzymology, vol. 185, Academic Press, Inc., San Diego, CA or Krieger (1990) Gene Transfer and Expression—A Laboratory Manual, Stockton Press, New York, N.Y. and the references cited therein).

The culture of cells used in the present disclosure, including cell lines and cultured cells from tissue or blood samples is well known in the art (see, e.g., Freshney (1994) Culture of Animal Cells, a Manual of Basic Technique, third edition, Wiley-Liss, N. Y. and the references cited therein).

Techniques for using and manipulating antibodies are found in Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, CA, and references cited therein; Goding (1986) Monoclonal Antibodies: Principles and Practice (2d ed.) Academic Press, New York, NY; and Kohler and Milstein (1975) Nature 256:495-497.

The anti-BoNT antibody gene(s) (e.g., anti-BoNT scFv gene) may be subcloned into the expression vector pUC119mycHis (Tomlinson et al. (1996) J. Mol. Biol., 256:813-817) or pSYN3, resulting in the addition of a hexahistidine tag at the C-terminal end of the scFv to facilitate purification. Detailed protocols for the cloning and purification of certain anti-BoNT antibodies are found, for example, in Amersdorfer et al. (1997) Infect. Immunity, 65 (9): 3743-3752, and the like.

### Preparation of Whole Polyclonal or Monoclonal Antibodies

The anti-BoNT antibodies of the present disclosure include individual, allelic, strain, or species variants, and fragments thereof, both in their naturally occurring (full-length) forms and in recombinant forms. Certain antibodies may be selected to bind one or more epitopes bound by the antibodies described herein. The antibodies can be raised in their native configurations or in non-native configurations. Anti-idiotypic antibodies can also be generated. Many methods of making antibodies that specifically bind to a particular epitope are known to persons of skill. The following discussion is presented as a general overview of the techniques available; however, one of skill will recognize that many variations upon the following methods are known.

### Polyclonal Antibody Production

Methods of producing polyclonal antibodies are known to those of skill in the art. In brief, an immunogen (e.g., BoNT/G, etc.), subsequences including, but not limited to subsequences comprising epitopes specifically bound by antibodies expressed by clones disclosed herein, e.g., a purified polypeptide, a polypeptide coupled to an appropriate carrier (e.g., GST, keyhole limpet hemanocyanin, etc.), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the BoNT polypeptide is performed where desired (see, e.g., Coligan (1991) Current Protocols in Immunology Wiley/Greene, NY; and Harlow and Lane (1989) Antibodies: A Laboratory Manual Cold Spring Harbor Press, NY).

Antibodies that specifically bind to the epitopes described herein can be selected from polyclonal sera using the selection techniques described herein.

### Monoclonal Antibody Production

In some instances, it is desirable to prepare monoclonal antibodies from various mammalian hosts, such as mice, rodents, primates, humans, etc. Descriptions of techniques for preparing such monoclonal antibodies are found in, e.g., Stites et al. (eds.) Basic and Clinical Immunology (4th ed.) Lange Medical Publications, Los Altos, CA, and references cited therein; Harlow and Lane, supra; Goding (1986) Monoclonal Antibodies: Principles and Practice (2d ed.) Academic Press, New York, NY; and Kohler and Milstein (1975) Nature 256:495-497.

Summarized briefly, monoclonal antibody production using hybridomas may proceed by injecting an animal with an immunogen (e.g., BoNT/G, etc.) subsequences including, but not limited to subsequences comprising epitopes specifically bound by antibodies expressed by clones disclosed herein. The animal is then sacrificed and cells taken from its spleen, which are fused with myeloma cells. The result is a hybrid cell or “hybridoma” that is capable of reproducing antibodies in vitro. The population of hybridomas is then screened to isolate individual clones, each of which secretes a single antibody species to the immunogen. In this manner, the individual antibody species obtained are the products of immortalized and cloned single B cells from the immune animal generated in response to a specific site recognized on the immunogenic substance.

Alternative methods of immortalization include transformation with Epstein Barr Virus, oncogenes, or retroviruses, or other methods known in the art. Colonies arising from single immortalized cells are screened for production of antibodies of the desired specificity and affinity for the BoNT antigen, and yield of the monoclonal antibodies produced by such cells is enhanced by various techniques, including injection into the peritoneal cavity of a vertebrate (e.g., mammalian) host. The antibodies of the present disclosure are used with or without modification, and include chimeric antibodies such as humanized murine antibodies.

Techniques for creating recombinant DNA versions of the antigen-binding regions of antibody molecules which bypass the generation of hybridomas are contemplated for the present BoNT binding antibodies and fragments. DNA is cloned into a bacterial expression system. One example of a suitable technique uses a bacteriophage lambda vector system having a leader sequence that causes the expressed Fab protein to migrate to the periplasmic space (between the bacterial cell membrane and the cell wall) or to be secreted. One can rapidly generate and screen great numbers of functional Fab fragments for those which bind BoNT. Such BoNT binding agents (Fab fragments with specificity for a BoNT polypeptide) are specifically encompassed within the BoNT binding antibodies and fragments of the present disclosure. Other methods for screening and production of antibodies may employ one or more of display systems such as phage display, yeast display, ribosome, etc., and an antibody production system such as that derived from transgenic mice.

The present disclosure provides the full amino acid sequence of a variable heavydomain in an antibody which can be easily reverse translated into a nucleotide sequence to encode the antibody of the present disclosure. The present disclosure provides the full amino acid sequence of V domain in a subject antibody which can be easily reverse translated into an isolated nucleic acid comprising a nucleotide sequence encoding an amino acid sequence of a VH of a subject antibody. The present disclosure provides the full amino acid sequence of V domain in a subject antibody which can be easily reverse translated into an isolated nucleic acid comprising a nucleotide sequence encoding an amino acid sequence of a V_{L} of a subject antibody. The present disclosure provides the full amino acid sequence of V domain in a subject antibody which can be easily reverse translated into an isolated nucleic acid comprising a nucleotide sequence encoding an amino acid sequence of a VH and a V_{L} of a subject antibody. In some instances, a subject nucleic acid comprises a nucleotide sequence encoding VH CDR1, CDR2, and CDR3 of a subject antibody and/or a V_{L} CDR1, CDR2, and CDR3 of a subject antibody.

In some cases, the isolated nucleic acid comprises the nucleotide sequence encoding an amino acid sequence of a) a VH of an antibody comprising a CDR1, CDR2 and CDR3, wherein the CDR1, CDR2, and CDR3 are selected from the VH of an antibody listed in Tables 1-6; and b) a V_{L} of an antibody comprising a CDR1, CDR2 and CDR3, wherein the CDR1, CDR2, and CDR3 are selected from the V_{L} of an antibody listed in Tables 1-6. In some cases, the antibody is the Hu6G6.2 antibody, the Hu6G7.2 antibody, the Hu6G9.1 antibody, the Hu6G10 antibody, or the Hu6G11.2 antibody.

The present disclosure provides an isolated nucleic acid comprising a nucleotide sequence encoding an amino acid sequence of a variable heavy chain (VH) polypeptide comprising a VH CDR1 selected from a VH of an antibody listed in Tables 1-6, a VH CDR2 comprising a VH CDR2 selected from a VH of the antibody an antibody listed in Tables 1-6, and a VH CDR2 selected from a VH of the antibody an antibody listed in Tables 1-6. In such cases, a variable heavy chain (VH) polypeptide is encoded by the nucleic acid. In some cases, the antibody is the Hu6G6.2 antibody, the Hu6G7.2 antibody, the Hu6G9.1 antibody, the Hu6G10 antibody, or the Hu6G11.2 antibody.

The present disclosure provides an isolated nucleic acid comprising a nucleotide sequence encoding an amino acid sequence of a variable light chain (V_{L}) polypeptide comprising a V_{L} CDR1 selected from a V_{L} of the antibody an antibody listed in Tables 1-6, a V_{L} CDR2 comprising a V_{L} CDR2 selected from a V_{L} of the antibody an antibody listed in Tables 1-6, and a V_{L} CDR2 selected from a V_{L} of the antibody an antibody listed in Tables 1-6. In such cases, the variable light chain (V_{L}) polypeptide is encoded by the nucleic acid. In some cases, the antibody is the Hu6G6.2 antibody, the Hu6G7.2 antibody, the Hu6G9.1 antibody, the Hu6G10 antibody, or the Hu6G11.2 antibody.

The nucleic acid can be a recombinant vector, as described above, which provides for amplification and/or expression (synthesis) of the encoded antibody. The recombinant vector can be suitable for expression in prokaryotic and/or eukaryotic cells.

The present disclosure also provides a cell, e.g., a genetically modified cell, that comprises a subject nucleic acid. A subject genetically modified cell can be a prokaryotic cell (e.g., a bacterial cell); or a eukaryotic cell (e.g., an insect cell; a mammalian cell, such as a mammalian cell line suitable for in vitro cell culture; a yeast cell; etc.), where the cell may produce the encoded antibody.

### IV. Modification of Anti-BoNT Antibodies

Creation of Anti-BoNT (scFv′) 2 Homodimers

To create anti-BoNT (scFv′) 2 antibodies, two anti-BoNT scFvs are joined, either through a linker (e.g., a carbon linker, a peptide, etc.) or through a disulfide bond between, for example, two cysteines. Thus, for example, to create disulfide linked scFv, a cysteine residue can be introduced by site directed mutagenesis between a myc tag and a hexahistidine tag at the carboxy-terminus of an anti-BoNT/G. Introduction of the correct sequence can be verified by DNA sequencing. The construct may be in pUC119, so that the pelB leader directs expressed scFv to the periplasm and cloning sites (Ncol and Notl) exist to introduce anti-BoNT mutant scFv. Expressed scFv has the myc tag at the C-terminus, followed by two glycines, a cysteine, and then 6 histidines to facilitate purification by IMAC. After disulfide bond formation between the two cysteine residues, the two scFv can be separated from each other by 26 amino acids (two 11 amino acid myc tags and three repeats of a unit with 4 glycines plus one serine). An scFv expressed from this construct, purified by IMAC may predominantly comprise monomeric scFv. To produce (scFv′)₂ dimers, the cysteine can be reduced by incubation with 1 mM beta-mercaptoethanol, and half of the scFv blocked by the addition of DTNB. Blocked and unblocked scFvs can be incubated together to form (scFv′); and the resulting material can optionally be analyzed by gel filtration. The affinity of the anti-BoNT scFv′ monomer and (scFv′) 2 dimer can optionally be determined by BIAcore.

The (scFv′) 2 dimer may be created by joining the scFv fragments through a linker, e.g., through a peptide linker. This can be accomplished by a wide variety of means well known to those of skill in the art. For example, one suitable approach is described by Holliger et al. (1993) Proc. Natl. Acad. Sci. USA, 90:6444-6448 (see also WO 94/13804).

Typically, linkers are introduced by PCR cloning. For example, synthetic oligonucleotides encoding the 5 amino acid linker (Gly₄Ser, SEQ ID NO: 224) can be used to PCR amplify the anti-BoNT antibody V_{H} and V_{L} genes which are then spliced together to create the anti-BoNT diabody gene. The gene can then be cloned into an appropriate vector, expressed, and purified according to standard methods well known to those of skill in the art.

Preparation of Anti-BoNT (scFv)₂, Fab, and (Fab′)₂ Molecules

Anti-BoNT antibodies such as anti-BoNT/G scFv, or variant(s) with higher affinity, are suitable templates for creating size and valency variants. For example, an anti-BoNT (scFv′)₂ can be created from the parent scFv as described above. An scFv gene can be excised using appropriate restriction enzymes and cloned into another vector as described herein.

Expressed scFv may include a myc tag at the C-terminus, followed by two glycines, a cysteine, and six histidines to facilitate purification. After disulfide bond formation between the two cystine residues, the two scFv may be separated from each other by 26 amino acids (e.g., two eleven amino acid myc tags and four glycines). Single-chain Fv (scFv) can be expressed from this construct and purified.

To produce (scFv′)₂ dimers, the cysteine is reduced by incubation with 1 mM β-mercaptoethanol, and half of the scFv blocked by the addition of DTNB. Blocked and unblocked scFv are incubated together to form (scFv′)₂, which is purified. As higher affinity scFv are isolated, their genes are similarly used to construct (scFv′)₂.

Anti-BoNT Fab may also be expressed in E. coli using an expression vector similar to the one described by Better et al. (1988) Science, 240:1041-1043. For example, to create a BoNT/G binding Fab, the V_{H} and V_{L} genes are amplified from the scFv using PCR. The VH gene is cloned into an expression vector (e.g., a pUC119 based bacterial expression vector) that provides an IgG C_{H}1 domain downstream from, and in frame with, the V_{H} gene. The vector also contains the lac promoter, a pelB leader sequence to direct expressed V_{H}-C_{H}1 domain into the periplasm, a gene 3 leader sequence to direct expressed light chain into the periplasm, and cloning sites for the light chain gene. Clones containing the correct V_{H} gene are identified, e.g., by PCR fingerprinting. The V_{L} gene is spliced to the C_{L} gene using PCR and cloned into the vector containing the V_{H}C_{H}1 gene.

### Selection of Antibodies

Selection of anti-BoNT antibodies (whether produced by phage display, yeast display, immunization methods, hybridoma technology, etc.) can involve screening the resulting antibodies for specific binding to an appropriate antigen(s). In the instant case, suitable antigens can include, but are not limited to BoNT/G, a C-terminal domain of BoNT heavy chain (binding domain) of BoNT holotoxins, recombinant BoNT domains such as He (binding domain), H_{N} (translocation domain), or Le (light chain), and the like. The antibodies may be selected for specific binding of an epitope recognized by one or more of the antibodies described herein.

Selection can be by any of a number of methods well known to those of skill in the art. In one example, selection is by immunochromatography (e.g., using immunotubes, Maxisorp, Nunc) against the desired target, e.g., BoNT/G, etc., In a related example, selection is against a BoNT protein in a surface plasmon resonance system (e.g., BIAcore, Pharmacia) either alone or in combination with an antibody that binds to an epitope specifically bound by one or more of the antibodies described herein. Selection can also be done using flow cytometry for yeast display libraries. Yeast display libraries are sequentially selected to obtain antibodies that bind with high affinity to all subtypes of BoNT/G if discovered later. This can be repeated for other subtypes.

For phage display, analysis of binding can be simplified by including an amber codon between the antibody fragment gene and gene III. This makes it possible to easily switch between displayed and soluble antibody fragments simply by changing the host bacterial strain. When phage are grown in a supE suppresser strain of E. coli, the amber stop codon between the antibody gene and gene III is read as glutamine and the antibody fragment is displayed on the surface of the phage. When eluted phage are used to infect a non-suppressor strain, the amber codon is read as a stop codon and soluble antibody is secreted from the bacteria into the periplasm and culture media (Hoogenboom et al. (1991) Nucleic Acids Res., 19:4133-4137). Binding of soluble scFv to antigen can be detected, e.g., by ELISA using a murine IgG monoclonal antibody (e.g., 9El0) which recognizes a C-terminal myc peptide tag on the scFv (Evan et al. (1985) Mol. Cell Biol., 5:3610-3616; Munro et al. (1986) Cell, 46:291-300), e.g., followed by incubation with polyclonal anti-mouse Fc conjugated to a detectable label (e.g., horseradish peroxidase).

As indicated above, purification of the anti-BoNT antibody can be facilitated by cloning of the scFv gene into an expression vector (e.g., expression vector pUC119mycHIS) that results in the addition of the myc peptide tag followed by a hexa-histidine tag at the C-terminal end of the scFv. The vector may also encode a pectate lyase leader sequence that directs expression of the scFv into the bacterial periplasm where the leader sequence is cleaved. This makes it possible to harvest native properly folded scFv directly from the bacterial periplasm. The anti-BoNT antibody is then expressed and purified from the bacterial supernatant using immobilized metal affinity chromatography.

### Measurement of Anti-BoNT Antibody Affinity for One or More BoNT Subtypes

As explained above, selection for increased avidity involves measuring the affinity of an anti-BoNT antibody (e.g., a modified anti-BoNT antibody) for one or more targets of interest (e.g., BoNT subtype(s) or domains thereof. For example, the K_{D} of a BoNT/G-binding antibody and the kinetics of binding to BoNT/G are determined in KinExA (Sapidyne Instruments, Inc., Boise, ID), a flow fluorimeter which allows true measurement of binding affinity and kinetics for unmodified molecules in solution, including antibody-antigen interactions. Samples with serious dilutions are drawn over solid-phase beads coupled with one binding partner to capture the complimentary binding molecule, which is detected by flow of fluorophore-labeled antibody solution binding to a separate epitope on the same molecular that being measured. Signals are used to calculate free concentration in solution after equilibrium is reached (i.e. to determine binding affinity KD), or under pre-equilibrium conditions over time (i.e. to determine binding kinetics kₒₙ or K_{off}). Kₒₙ is typically measured in the range 1.0×10² to 5.0×10⁶ M and k_{off} in the range 1.0×10⁻¹ to 1.0×10⁻⁶ M. The equilibrium constant K_{d} is then calculated as K_{off}/kₒₙ and thus is typically measured in the range 10⁻⁵ to 10⁻¹² M. Affinities measured in this manner usually correlate well with affinities measured in solution by fluorescence quench titration.

### V. Humanized, Human Engineered or Human Antibody Production

The present BoNT binding antibodies and fragments can be humanized or human engineered antibodies. As used herein, a humanized antibody, or antigen binding fragment thereof, is a recombinant polypeptide that comprises a portion of an antigen binding site from a non-human antibody and a portion of the framework and/or constant regions of a human antibody. A human engineered antibody or antibody fragment may be derived from a human or non-human (e.g., mouse) source that has been engineered by modifying (e.g., deleting, inserting, or substituting) amino acids at specific positions so as to alter certain biophysical properties or to reduce any detectable immunogenicity of the modified antibody in a human.

Humanized antibodies also encompass chimeric antibodies and CDR-grafted antibodies in which various regions may be derived from different species. Chimeric antibodies may be antibodies that include a non-human antibody variable region linked to a human constant region. Thus, in chimeric antibodies, the variable region is mostly non-human, and the constant region is human. Chimeric antibodies and methods for making them are described in Morrison, et al., Proc. Natl. Acad. Sci. USA, 81: 6841-6855 (1984), Boulianne, et al., Nature, 312: 643-646 (1984), and PCT Application Publication WO 86/01533. Although, they can be less immunogenic than a mouse monoclonal antibody, administrations of chimeric antibodies have been associated with human anti-mouse antibody responses (HAMA) to the non-human portion of the antibodies. Chimeric antibodies can also be produced by splicing the genes from a mouse antibody molecule of appropriate antigen-binding specificity together with genes from a human antibody molecule of appropriate biological activity, such as the ability to activate human complement and mediate ADCC. Morrison et al. (1984), Proc. Natl. Acad. Sci., 81:6851; Neuberger et al. (1984), Nature, 312:604. One example is the replacement of an Fc region with that of a different isotype.

CDR-grafted antibodies are antibodies that include the CDRs from a non-human “donor” antibody linked to the framework region from a human “recipient” antibody. Generally, CDR-grafted antibodies include more human antibody sequences than chimeric antibodies because they include both constant region sequences and variable region (framework) sequences from human antibodies. Thus, for example, a CDR-grafted humanized antibody may comprise a heavy chain that comprises a contiguous amino acid sequence (e.g., about 5 or more, 10 or more, or even 15 or more contiguous amino acid residues) from the framework region of a human antibody (e.g., FR-1, FR-2, or FR-3 of a human antibody) or, optionally, most or all of the entire framework region of a human antibody. CDR-grafted antibodies and methods for making them are described in, Jones et al., Nature, 321:522-525 (1986), Riechmann et al., Nature, 332:323-327 (1988), and Verhoeyen et al., Science, 239:1534-1536 (1988)). Methods that can be used to produce humanized antibodies also are described in U.S. Pat. Nos. 4,816,567, 5,721,367, 5,837,243, and 6,180,377. CDR-grafted antibodies are considered less likely than chimeric antibodies to induce an immune reaction against non-human antibody portions. However, it has been reported that framework sequences from the donor antibodies are required for the binding affinity and/or specificity of the donor antibody, presumably because these framework sequences affect the folding of the antigen-binding portion of the donor antibody. Therefore, when donor, non-human CDR sequences are grafted onto unaltered human framework sequences, the resulting CDR-grafted antibody can exhibit, in some cases, loss of binding avidity relative to the original non-human donor antibody. See, e.g., Riechmann et al., Nature, 332: 323-327 (1988), and Verhoeyen et al., Science, 239:1534-1536 (1988).

Human engineered antibodies include for example “veneered” antibodies and antibodies prepared using HUMAN ENGINEERING™ technology (U.S. Pat. No. 5,869,619). HUMAN ENGINEERING™ technology is commercially available, and involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Techniques for making human engineered proteins are described in Studnicka et al., Protein Engineering, 7: 805-814 (1994), U.S. Pat. Nos. 5,766,886, 5,770,196, 5,821,123, and 5,869,619, and PCT Application Publication WO 93/11794.

“Veneered” antibodies are non-human or humanized (e.g., chimeric or CDR-grafted antibodies) antibodies that have been engineered to replace certain solvent-exposed amino acid residues so as to further reduce their immunogenicity or enhance their function. As surface residues of a chimeric antibody are presumed to be less likely to affect proper antibody folding and more likely to elicit an immune reaction, veneering of a chimeric antibody can include, for instance, identifying solvent-exposed residues in the non-human framework region of a chimeric antibody and replacing at least one of them with the corresponding surface residues from a human framework region. Veneering can be accomplished by any suitable engineering technique, including the use of the above-described HUMAN ENGINEERING™ technology.

In a different approach, a recovery of binding avidity can be achieved by “de-humanizing” a CDR-grafted antibody. De-humanizing can include restoring residues from the donor antibody's framework regions to the CDR grafted antibody, thereby restoring proper folding. Similar “de-humanization” can be achieved by (i) including portions of the “donor” framework region in the “recipient” antibody or (ii) grafting portions of the “donor” antibody framework region into the recipient antibody (along with the grafted donor CDRs).

For a further discussion of antibodies, humanized antibodies, human engineered, and methods for their preparation, see Kontermann and Dubel, eds., Antibody Engineering, Springer, New York, NY, 2001.

The present antibodies and fragments encompass antibodies having CDRs of human origin, such as antibodies which bind BoNT polypeptides and are encoded by nucleic acid sequences which are naturally occurring somatic variants of human germline immunoglobulin nucleic acid sequence, and fragments, synthetic variants, derivatives and fusions thereof. Such antibodies may be produced by any method known in the art, such as through the use of transgenic mammals (such as transgenic mice) in which the native immunoglobulin repertoire has been replaced with human V-genes in the mammal chromosome. Such mammals appear to carry out VDJ recombination and somatic hypermutation of the human germlinc antibody gencs in a normal fashion, thus producing high affinity antibodies with completely human sequences.

Human antibodies can also be generated through the in vitro screening of antibody display libraries. See Hoogenboom et al. (1991), J. Mol. Biol. 227:381; and Marks et al. (1991), J. Mol. Biol. 222:581. Various antibody-containing phage display libraries have been described and may be readily prepared. Libraries may contain a diversity of human antibody sequences, such as human Fab, Fv, and scFv fragments that may be screened against an appropriate target. Phage display libraries may comprise peptides or proteins other than antibodies which may be screened to identify selective binding agents of BoNT.

The development of technologies for making repertoires of recombinant human antibody genes, and the display of the encoded antibody fragments on the surface of filamentous bacteriophage, has provided a means for making human antibodies directly. The antibodies produced by phage technology are produced as antigen binding fragments-usually Fv or Fab fragments-in bacteria and thus lack effector functions. Effector functions can be introduced by one of two strategies: The fragments can be engineered either into complete antibodies for expression in mammalian cells, or into bispecific antibody fragments with a second binding site capable of triggering an effector function.

Methods for display of peptides on the surface of yeast and microbial cells have also been used to identify antigen specific antibodies. See, for example, U.S. Pat. No. 6,699,658. Antibody libraries may be attached to yeast proteins, such as agglutinin, effectively mimicking the cell surface display of antibodies by B cells in the immune system.

In addition to phage display methods, antibodies may be isolated using ribosome mRNA display methods and microbial cell display methods. Selection of polypeptide using ribosome display is described in Hanes et al., (Proc. Nat.l Acad. Sc.i USA, 94:4937-4942, 1997) and U.S. Pat. Nos. 5,643,768 and 5,658,754 issued to Kawasaki. Ribosome display is also useful for rapid large scale mutational analysis of antibodies. The selective mutagenesis approach also provides a method of producing antibodies with improved activities that can be selected using ribosomal display techniques.

### V_{L} Other Antibody Forms

Sequence provided herein can be used to generate other antibody forms, including but not limited to nanobodies, UniBodies, and/or affibodies.

VHH and/or Nanobodies

The Camelidae heavy chain antibodies are found as homodimers of a single heavy chain, dimerized via their constant regions. The variable domains of these camelidae heavy chain antibodies are referred to as VHH domains or VHH, and can be either used per se as nanobodies and/or as a starting point for obtaining nanobodies. Isolated VHH retain the ability to bind antigen with high specificity (see, e.g., Hamers-Casterman et al. (1993) Nature 363:446-448). VHH domains, or nucleotide sequences encoding them, can be derived from antibodies raised in Camelidae species, for example in camel, dromedary, llama, alpaca and guanaco. Other species besides Camelidae (e.g, shark, pufferfish) can produce functional antigen-binding heavy chain antibodies, from which (nucleotide sequences encoding) such naturally occurring VHH can be obtained, e.g., using the methods described in U.S. Patent Publication US 2006/0211088.

Human proteins may be used in therapy primarily because they are not as likely to provoke an immune response when administered to a patient. Comparisons of camelid VHH with the V_{H} domains of human antibodies reveals several key differences in the framework regions of the camelid VHH domain corresponding to the V_{H}/V_{L} interface of the human V_{H} domains. Mutation of these human residues to VHH resembling residues has been performed to produce “camelized” human V_{H} domains that retain antigen binding activity, yet have improved expression and solubility.

Libraries of single V_{H} domains have also been derived for example from V_{H} genes amplified from genomic DNA or from mRNA came from the spleens of immunized mice and expressed in E. coli (Ward et al. (1989) Nature 341:544-546) and similar approaches can be performed using the V_{H} domains and/or the V_{L} domains described herein. The isolated single V_{H} domains are called “dAbs” or domain antibodies. A “dAb” is an antibody single variable domain (V_{H} or V_{L}) polypeptide that specifically binds antigen. A “dAb” binds antigen independently of other V domains; however, as the term is used herein, a “dAb” can be present in a homo- or heteromultimer with other V_{H} or V_{L} domains where the other domains are not required for antigen binding by the dAb, i.e., where the dAb binds antigen independently of the additional V_{H} or V_{L} domains.

As described in U.S. Patent Publication No. 2006/0211088 methods are known for the cloning and direct screening of immunoglobulin sequences (including but not limited to multivalent polypeptides comprising: two or more variable domains—or antigen binding domains—and in particular V_{H} domains or VHH domains; fragments of V_{L}, V_{H} or VHH domains, such as CDR regions, for example CDR3 regions; antigen-binding fragments of conventional 4-chain antibodies such as Fab fragments and scFv's, heavy chain antibodies and domain antibodies; and in particular of VH sequences, and more in particular of VHH sequences) that can be used as part of and/or to construct such nanobodies.

Methods and procedures for the production of VHH/nanobodies can also be found for example in WO 94/04678, WO 96/34103, WO 97/49805, WO 97/49805 WO 94/25591, WO 00/43507 WO 01/90190, WO 03/025020, WO 04/062551, WO 04/041863, WO 04/041865, WO 04/041862, WO 04/041867, PCT/BE2004/000159, Hamers-Casterman et al. (1993) Nature 363:446; Riechmann and Muyldermans (1999) J. Immunological Meth., 231:25-38; Vu et al. (1997) Molecular Immunology, 34(16-17): 1121-1131; Nguyen et al. (2000) EMBO J., 19 (5): 921-930; Arbabi Ghahroudi et al. (19997) FEBS Letters 414:521-526; van der Linden et al. (2000) J. Immunological Meth., 240:185-195; Muyldermans (2001) Rev. Molecular Biotechnology 74:277-302; Nguyen el al. (2001) Adv. Immunol. 79:261, and the like, which are all incorporated herein by reference.

### UniBodies

UniBodies are generated by an antibody technology that produces a stable, smaller antibody format with an anticipated longer therapeutic window than certain small antibody formats. UniBodies may be produced from IgG4 antibodies by eliminating the hinge region of the antibody. Unlike the full size IgG4 antibody, the half molecule fragment is very stable and is termed a UniBody. Halving the IgG4 molecule left only one area on the UniBody that can bind to a target. Methods of producing UniBodies are described in detail in PCT Publication WO2007/059782, which is incorporated herein by reference in its entirety (see, also, Kolfschoten et al. (2007) Science 317:1554-1557).

### Affibodies

Affibody molecules are class of affinity proteins based on a 58-amino acid residue protein domain, derived from one of the IgG-binding domains of staphylococcal protein A. This three helix bundle domain has been used as a scaffold for the construction of combinatorial phagemid libraries, from which affibody variants that target the desired molecules can be selected using phage display technology (see, e.g., Nord et al. (1997) Nat. Biotechnol. 15:772-777; Ronmark et al. (2002) Eur. J. Biochem., 269:2647-2655.). Details of affibodies and methods of production are known to those of skill (see, e.g., U.S. Pat. No. 5,831,012 which is incorporated herein by reference in its entirety).

### VII. Assaying for Cross-Reactivity at an Epitope

The antibodies of the present disclosure encompass those that specifically bind to one or more epitopes recognized by antibodies listed in Tables 1-6. In other words, antibodies are cross-reactive with one or more of these epitopes but may have different sequences. Means of assaying for cross-reactivity are well known to those of skill in the art (see, e.g., Dowbenko et al. (1988) J. Virol. 62:4703-4711).

This can be ascertained by providing one or more isolated target BoNT polypeptide(s) (e.g., BoNT/G or recombinant domains of said toxin, such as Hc) attached to a solid support and assaying the ability of a test antibody to compete with an antibody described herein for binding to the target BoNT peptide. Thus, immunoassays in a competitive binding format can be used for cross-reactivity determinations. For example, a BoNT/G polypeptide may be immobilized to a solid support. Antibodies to be tested (e.g., generated by selection from a phage-display library) added to the assay compete with any antibody listed in Table 4 for binding to the immobilized BoNT polypeptide(s). The ability of test antibodies to compete with the binding of one or more antibodies listed in Tables 1-6 to the immobilized protein(s) are compared. The percent cross-reactivity above proteins is then calculated, using standard calculations.

If the test antibody competes with one or more of the antibodies listed in Tables 1-6 and has a binding affinity comparable to or greater than a threshold (such as having a Kp equal or less than about 1×10⁻⁸ M) with the same target then the test antibody is expected to be an anti-BoNT antibody. In some cases, a subject antibody competes for binding to a Botulinum neurotoxin epitope with an antibody comprising V_{H} and/or V_{L} CDRs (e.g., V_{H} CDR1, CDR2, and CDR3; and/or V_{L} CDR1, CDR2, and CDR3) of an antibody depicted in Tables 1-6. As one non-limiting example, in some instances, a subject antibody competes for binding to a Botulinum neurotoxin epitope with an antibody comprising VH CDR1, VH CDR2, and VH CDR3 of the antibody designated Hu6G6.2. In some instances, a subject antibody competes for binding to a Botulinum neurotoxin epitope with an antibody comprising VL CDR1, VL CDR2, and VL CDR3 of the antibody designated Hu6G6.2. As another example, in some cases, a subject antibody competes for binding to a Botulinum neurotoxin epitope with an antibody comprising VH CDR1, VH CDR2, VH CDR3, VL CDR1, VL CDR2, VL CDR3 of the antibody designated Hu6G6.2.

Cross-reactivity may be performed by using surface plasmon resonance in a BIAcore. In a BIAcore flow cell, the BoNT polypeptide(s) (e.g., BoNT/G) are coupled to a sensor chip (e.g., CM5) as described in WO 09/008916, disclosure of which is incorporated herein by reference. With a flow rate of 5 μl/min, a titration of 100 nM to 1 μM antibody is injected over the flow cell surface for about 5 minutes to determine an antibody concentration that results in near saturation of the surface. Epitope mapping is then evaluated using pairs of antibodies at concentrations resulting in near saturation and at least 100 relative units (RU) of antibody bound. The amount of antibody bound is determined for each member of a pair, and then the two antibodies are mixed together to give a final concentration equal to the concentration used for measurements of the individual antibodies. Antibodies recognizing different epitopes show an essentially additive increase in the RU bound when injected together, while antibodies recognizing identical epitopes show only a minimal increase in RU. Antibodies may be said to be cross-reactive if, when “injected” together they show an essentially additive increase (e.g., an increase by at least a factor of about 1.4, an increase by at least a factor of about 1.6, or an increase by at least a factor of about 1.8 or 2).

Epitope mapping may also be determined by incubating a yeast displayed scFv with a BoNT domain polypeptide followed by incubation with an epitope-tagged scFv. Bound scFv is detected with an antibody recognizing the epitope tag and the level of BoNT domain display quantitated by incubation with anti-SV5.

Cross-reactivity at the desired epitopes can be ascertained by a number of other standard techniques (see, e.g., Geysen et al (1987) J. Immunol. Meth. 102, 259-274). This technique involves the synthesis of large numbers of overlapping BoNT peptides. The synthesized peptides are then screened against one or more of the prototypical antibodies (e.g., the Hu6G6.2 antibody, the Hu6G7.2 antibody, the Hu6G9.1 antibody, the Hu6G10 antibody, or the Hu6G11.2 antibody, etc.) and the characteristic epitopes specifically bound by these antibodies can be identified by binding specificity and affinity. The epitopes thus identified can be conveniently used for competitive assays as described herein to identify cross-reacting antibodies.

The peptides for epitope mapping can be conveniently prepared using “Multipin” peptide synthesis techniques (see, e.g., Geysen et al (1987) Science, 235:1184-1190). Using the known sequence of one or more BoNT subtypes (see. e.g., Atassi et al. (1996) J. Prot. Chem., 7:691-700 and references cited therein), overlapping BoNT polypeptide sequences can be synthesized individually in a sequential manner on plastic pins in an array of one or more 96-well microtest plate(s).

The procedure for epitope mapping using this multipin peptide system is described in U.S. Pat. No. 5,739,306. Briefly, the pins are first treated with a pre-coat buffer containing 2% bovine serum albumin and 0.1% Tween 20 in phosphate-buffered saline (PBS) for 1 hour at room temperature. Then the pins are then inserted into the individual wells of 96-well microtest plate containing the antibodies in the pre-coat buffer, e.g., at 2 μg/ml. The incubation can be carried out for about 1 hour at room temperature. The pins are washed in PBST (e.g., 3 rinses for every 10 minutes), and then incubated in the wells of a 96-well microtest plate containing 100 μl of horse radish peroxidase (HRP)-conjugated goat anti-mouse IgG (Fc) (Jackson ImmunoResearch Laboratories) at a 1:4,000 dilution for 1 hour at room temperature. After the pins are washed as before, the pins are put into wells containing peroxidase substrate solution of diammonium 2,2′-azino-bis [3-ethylbenzthiazoline-b-sulfonate] (ABTS) and H₂O₂ (Kirkegaard & Perry Laboratories Inc., Gaithersburg, Md.) for 30 minutes at room temperature for color reaction. The plate is read at 405 nm by a plate reader (e.g., BioTek ELISA plate reader) against a background absorption wavelength of 492 nm. Wells showing color development indicate reactivity of the BoNT peptides in such wells with the test antibodies.

### VIII. Assaying for Specific Binding and Neutralizing Activity of Anti-BoNT Antibodies

Exemplary antibodies of the present disclosure act, individually or in combination, to specifically bind to, and in some embodiments, neutralize (reduce or eliminate) the toxicity of botulinum neurotoxin type. Neutralization can be evaluated in vivo or in vitro. In vivo neutralization measurements simply involve measuring changes in the lethality (e.g., LD₅₀ or other standard metric) due to a BoNT neurotoxin administration with the presence of one or more antibodies being tested for neutralizing activity. The neurotoxin can be directly administered to the test organism (e.g., mouse) or the organism can harbor a botulism infection (e.g., be infected with Clostridium botulinum). The antibody can be administered before, during, or after the injection of BoNT neurotoxin or infection of the test animal. A decrease in the rate of progression, or mortality rate indicates that the antibody(s) have neutralizing activity.

One suitable in vitro assay for neutralizing activity uses a hemidiaphragm preparation (Deshpande et al. (1995) Toxicon, 33:551-557). Briefly, left and right phrenic nerve hemidiaphragm preparations are suspended in physiological solution and maintained at a constant temperature (e.g., 36° C.). The phrenic nerves are stimulated supramaximally (e.g., at 0.05 Hz with square waves of 0.2 ms duration). Isometric twitch tension is measured with a force displacement transducer (e.g., GrassModel FT03) connected to a chart recorder.

Purified antibodies are incubated with purified BoNT (e.g., BoNT/G, etc.) for 30 min at room temperature and then added to the tissue bath, resulting in a final antibody concentration of about 2.0×10⁻⁸ M and a final BoNT concentration of about 2.0×10⁻¹¹ M. For each antibody studied, time to 50% twitch tension reduction is determined (e.g., three times for BoNT alone and three times for antibody plus BoNT). Differences between times to a given (arbitrary) percentage (e.g., 50%) twitch reduction are determined by standard statistical analyses (e.g., two-tailed t test) at standard levels of significance (e.g., a P value of <0.05 considered significant).

### IX. Diagnostic Assays

As explained above, the anti-BoNT antibodies of the present disclosure can be used for the in vivo or in vitro detection of BoNT toxin and thus, are useful in the diagnosis (e.g., confirmatory diagnosis) of botulism. The detection and/or quantification of BoNT in a biological sample obtained from an organism is indicative of a Clostridium botulinum infection of that organism or intoxication of BoNT.

The BoNT antigen can be quantified in a biological sample derived from a patient such as a cell, or a tissue sample derived from a patient. As used herein, a biological sample is a sample of biological tissue or fluid that contains a BoNT concentration that may be correlated with and indicative of a Clostridium botulinum infection. Examples of suitable biological samples include blood (or blood fraction such as serum or plasma), urine, saliva, and tissue biopsies.

Although the sample is typically taken from a human patient, the assays can be used to detect BoNT antigen in samples from mammals in general, such as dogs, cats, sheep, cattle and pigs, and most particularly primates such as humans, chimpanzees, gorillas, macaques, and baboons, and rodents such as mice, rats, and guinea pigs.

Tissue or fluid samples are isolated from a patient according to standard methods well known to those of skill in the art, most typically by biopsy or venipuncture. The sample is optionally pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

### Immunological Binding Assays

The BoNT polypeptide (e.g., BoNT/G, etc.) can be detected in an immunoassay utilizing only one or more than one of the anti-BoNT antibodies of the present disclosure as a capture agent that specifically binds to the BoNT polypeptide.

As used herein, an immunoassay is an assay that utilizes only one or more than one antibody (e.g., one or more anti-BoNT antibodies disclosed herein) to specifically bind an analyte. The immunoassay is characterized by the binding of only one or more than one type of anti-BoNT antibody to a target (e.g., one or more BoNT/G epitopes) as opposed to other physical or chemical properties to isolate, target, and quantify the BoNT analyte.

The BoNT marker can be detected and quantified using any of a number of well recognized immunological binding assays. For example, the antibody of the present disclosure may be immobilized on a substrate (e.g., bead) and/or be the capture antibody in an ELISA. The detection step may take one of many formats known in the art, such as using a labeled secondary antibody or PCR amplification. Where PCR amplification is the method of detection, the antibody is conjugated to a nucleic acid, the antigen may optionally be first attached to a substrate, and the antibody is allowed to be bound to the antigen. The bound antibody-nucleic acid fusion then undergoes PCR amplification of the nucleic acid sequence attached to the antibody. The amplified sequences can in turn be detected via a fluorophore bound to the incorporated nucleotides. The amplified sequences can also be first hybridized to an array before fluorescence is measured to enable multiplexing. Multiplexing encompasses processing and detecting two or more samples and/or two or more analytes in parallel. Details of an assay using antibody-nucleic acid fusion may be found in US20060141505, disclosure of which is incorporated by reference.

Single assay or multiplex assay can also take the form of an array where signal is detected only by electro-stimulation. In this format, the antibody of the present disclosure is conjugated to an electrochemiluminescent moiety and immobilized on an electrode. A signal (e.g., fluorescence) is emitted due to electrical stimulation at a particular electrode. Details of an assay using electrochemiluminescent moiety in an array may be found in US20100140086, disclosure of which is incorporated by reference.

A fluorescent compound may be also added later to the assay for visualization by either Luminex type or other type of detection (see, e.g., U.S. Pat. Nos. 4,366,241; 4,376,110; 4,517,288; and 4,837,168, and the like). For a review of the general immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991)).

The immunoassays of the present disclosure can be performed in any of a number of configurations (see, e.g., those reviewed in Maggio (ed.) (1980) Enzyme Immunoassay CRC Press, Boca Raton, Florida; Tijan (1985) “Practice and Theory of Enzyme Immunoassays,” Laboratory Techniques in Biochemistry and Molecular Biology, Elsevier Science Publishers B.V., Amsterdam; Harlow and Lane, supra; Chan (ed.) (1987) Immunoassay: A Practical Guide Academic Press, Orlando, FL; Price and Newman (eds.) (1991) Principles and Practice of Immunoassays Stockton Press, NY; and Ngo (ed.) (1988) Non isotopic Immunoassays Plenum Press, NY).

Immunoassays often utilize a labeling agent to specifically bind to and label the binding complex formed by the capture agent and the analyte (e.g., an anti-BoNT/G antibody/BoNT/G complex). The labeling agent can itself be one of the moieties comprising the antibody/analyte complex. Thus, for example, the labeling agent can be a labeled BoNT/G polypeptide or a labeled anti-BoNT/G antibody. Alternatively, the labeling agent is optionally a third moiety, such as another antibody, that specifically binds to the BoNT antibody, the BoNT peptide(s), the antibody/polypeptide complex, or to a modified capture group (e.g., biotin) which is covalently linked to BoNT polypeptide or to the anti-BoNT antibody.

The labeling agent encompasses an antibody that specifically binds to the anti-BoNT antibody. Such agents are well known to those of skill in the art, and most typically comprise labeled antibodies that specifically bind antibodies of the particular animal species from which the anti-BoNT antibody is derived (e.g., an anti-species antibody). Thus, for example, where the capture agent is a human derived BoNT/G antibody, the label agent may be a mouse anti-human IgG, i.e., an antibody specific to the constant region of the human antibody.

Other proteins capable of specifically binding immunoglobulin constant regions, such as streptococcal protein A or protein G are also used as the labeling agent. These proteins are normal constituents of the cell walls of streptococcal bacteria. They exhibit a strong non-immunogenic reactivity with immunoglobulin constant regions from a variety of species (see generally Kronval, et al., (1973) J. Immunol., 111:1401-1406, and Akerstrom, et al., (1985) J. Immunol., 135:2589-2542, and the like).

Throughout the assays, incubation and/or washing steps may be required after each combination of reagents. Incubation steps can vary from about 5 seconds to several hours, for example, from about 5 minutes to about 24 hours (e.g., from 5 minutes to 15 minutes, from 15 minutes to 30 minutes, from 30 minutes to 60 minutes, from 1 hour to 4 hours, from 4 hours to 8 hours, from 8 hours to 12 hours, or from 12 hours to 24 hours). However, the incubation time will depend upon the assay format, analyte, volume of solution, concentrations, and the like. Usually, the assays are carried out at ambient temperature, although they can be conducted over a range of temperatures, such as 5° C. to 45° C.

### Non Competitive Assay Formats

Immunoassays for detecting BoNT neurotoxins (e.g., BoNT serotypes and/or subtypes) may be either competitive or noncompetitive. Noncompetitive immunoassays are assays in which the amount of captured analyte (in this case, BoNT/G polypeptide) is directly measured. In one example of a suitable “sandwich” assay, the capture agent (e.g., an anti-BoNT/G antibody) is bound directly or indirectly to a solid substrate where it is immobilized. These immobilized anti-BoNT/G antibodies capture BoNT polypeptide(s) present in a test sample (e.g., a blood sample). The BoNT polypeptide(s) thus immobilized are then bound by a labeling agent, e.g., an anti-BoNT/G antibody bearing a label. Alternatively, the second antibody may lack a label, but it may, in turn, be bound by a labeled third antibody specific to antibodies of the species from which the second antibody is derived. Free labeled antibody is washed away and the remaining bound labeled antibody is detected (e.g., using a gamma detector where the label is radioactive).

### Competitive Assay Formats

In competitive assays, the amount of analyte (e.g., BoNT/G) present in the sample is measured indirectly by measuring the amount of an added (exogenous) analyte displaced (or competed away) from a capture agent (e.g., anti-BoNT/G antibody) by the analyte present in the sample. For example, in one competitive assay, a known amount of BoNT/G is added to a test sample with an unquantified amount of BoNT, and the sample is contacted with a capture agent, e.g., an anti-BoNT antibody that specifically binds BoNT/G. The amount of added BoNT that binds to the anti-BoNT antibody is inversely proportional to the concentration of BoNT/G present in the test sample.

The anti-BoNT antibody can be immobilized on a solid substrate. The amount of BoNT/G bound to the anti-BoNT antibody is determined either by measuring the amount of BoNT present in a BoNT/G-anti-BoNT antibody complex, or alternatively by measuring the amount of remaining uncomplexed BoNT/G.

### Reduction of Non-Specific Binding

One of skill will appreciate that it is often desirable to reduce non-specific binding in immunoassays and during analyte purification. Where the assay involves, for example BoNT/G polypeptide(s), BoNT/G-binding antibody, or other capture agent(s) immobilized on a solid substrate, it is desirable to minimize the amount of non-specific binding to the substrate. Means of reducing such non-specific binding are well known to those of skill in the art. Typically, this involves coating the substrate with a protcinaccous composition. In particular, protein compositions such as bovine serum albumin (BSA), nonfat powdered milk, and gelatin are widely used.

### Substrates

As mentioned above, depending upon the assay, various components, including the BoNT/G polypeptide(s), anti-BoNT/G antibodies, etc., are optionally bound to a solid surface. Many methods for immobilizing biomolecules to a variety of solid surfaces are known in the art. For instance, the solid surface may be a membrane (e.g., nitrocellulose), a microtiter dish (e.g., PVC, polypropylene, or polystyrene), a test tube (glass or plastic), a dipstick (e.g., glass, PVC, polypropylene, polystyrene, latex, and the like), a microcentrifuge tube, or a glass, silica, plastic, metallic or polymer bead. The desired component may be covalently bound, or noncovalently attached through nonspecific bonding.

A wide variety of organic and inorganic polymers, both natural and synthetic may be employed as the material for the solid surface. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials which may be employed include paper, glasses, ceramics, metals, metalloids, semiconductive materials, cements or the like. In addition, substances that form gels, such as proteins (e.g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes may be employed depending upon the nature of the system.

In preparing the surface, a plurality of different materials may be employed, e.g., as laminates, to obtain various properties. For example, protein coatings, such as gelatin can be used to avoid non-specific binding, simplify covalent conjugation, and enhance signal detection or the like.

If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. See, for example, Immobilized Enzymes, Ichiro Chibata, Halsted Press, New York, 1978, and Cuatrecasas, (1970) J. Biol. Chem. 245 3059.

In addition to covalent bonding, various methods for noncovalently binding an assay component can be used. Noncovalent binding is typically nonspecific absorption of a compound to the surface. Typically, the surface is blocked with a second compound to prevent nonspecific binding of labeled assay components. Alternatively, the surface is designed such that it nonspecifically binds one component but does not significantly bind another. For example, a surface bearing a lectin such as concanavalin A will bind a carbohydrate containing compound but not a labeled protein that lacks glycosylation. Various solid surfaces for use in noncovalent attachment of assay components are reviewed in U.S. Pat. Nos. 4,447,576 and 4,254,082, which is incorporated herein by reference.

### Other Assay Formats

BoNT polypeptides or anti-BoNT antibodies (e.g., BoNT neutralizing antibodies and antibodies that specifically bind to BoNT/G) can also be detected and quantified by any of a number of other means well known to those of skill in the art. These include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like.

Western blot analysis and related methods can also be used to detect and quantify the presence of BoNT polypeptides in a sample. The technique generally comprises separating sample products by gel electrophoresis on the basis of molecular weight, transferring the separated products to a suitable solid support, (such as a nitrocellulose filter, a nylon filter, or derivatized nylon filter), and incubating the sample with the antibodies that specifically bind either the BoNT/G polypeptide. The antibodies specifically bind to the biological agent of interest on the solid support. These antibodies are directly labeled or alternatively are subsequently detected using labeled antibodies (e.g., labeled sheep anti-human antibodies where the antibody to a marker gene is a human antibody) which specifically bind to the antibody which binds the BoNT/G polypeptide.

Other assay formats include liposome immunoassays (LIAs), which use liposomes designed to bind specific molecules (e.g., antibodies) and release encapsulated reagents or markers. The released chemicals are then detected according to standard techniques (see, Monroe et al., (1986) Amer. Clin. Prod. Rev. 5:34-41).

### Labeling of Anti-BoNT/G Antibodies

Anti-BoNT/G antibodies can be labeled by any of a number of methods known to those of skill in the art. Thus, for example, the labeling agent can be, e.g., a monoclonal antibody, a polyclonal antibody, a protein or complex such as those described herein, or a polymer such as an affinity matrix, carbohydrate or lipid. Detection proceeds by any known method, including immunoblotting, western analysis, gel-mobility shift assays, tracking of radioactive or bioluminescent markers, nuclear magnetic resonance, electron paramagnetic resonance, stopped-flow spectroscopy, column chromatography, capillary electrophoresis, or other methods which track a molecule based upon an alteration in size and/or charge. The detectable group can be any material having a detectable physical or chemical property. Such detectable labels have been well-developed in the field of immunoassays and, in general, any label useful in such methods can be applied in the various embodiments of the present disclosure. Thus, a label is any composition detectable by spectroscopic, photochemical, biochemical, immunochemical, electrical, optical or chemical means. Useful labels in the present disclosure include magnetic beads (e.g., Dynabeads™), fluorescent dyes (e.g., fluorescein isothiocyanate, Texas red, rhodamine, Alexa fluor dyes and the like), radiolabels (e.g., ³H, ¹²⁵I, ³⁵S, ¹⁴C, or ³²P), enzymes (e.g., LacZ, CAT, horse radish peroxidase, luciferase, alkaline phosphatase and others, commonly used as detectable enzymes, either as marker gene products or in an ELISA), and colorimetric labels such as colloidal gold or colored glass or plastic (e.g., polystyrene, polypropylene, latex, etc.) beads. For example, an antibody can include a fluorescent label, a chemiluminescent label, a radiolabel, a chromogenic label, or other suitable label.

The label may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904, which is incorporated herein by reference.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of BoNT/G peptides. In this case, antigen-coated particles are agglutinated by samples comprising the target antibodies. In this format, none of the components need be labeled and the presence of the target antibody is detected by simple visual inspection.

### X. Compositions

The BoNT/G-binding antibodies of this disclosure are useful in preventing or mitigating the progression of botulism produced, e.g., by endogenous disease processes or by chemical/biological warfare agents. Typically, compositions containing one, two, or more different antibodies can be provided as a pharmaceutical composition and administered to a mammal (e.g., to a human) in need thereof.

When combined, different neutralizing antibodies exhibit a potency that is increased dramatically. This increase makes it possible to generate a botulinum antibody composition of the required potency for therapeutic use. Compositions comprising at least two, at least three, or more high affinity antibodies that bind overlapping or non-overlapping epitopes on the BoNT/G are contemplated herein.

As disclosed herein, particularly efficient neutralization of a botulism neurotoxin (BoNT/G) can be achieved by the use of antibodies that bind two or more BoNT epitopes with high affinity. This can be accomplished by using one, two or more different antibodies. Where there is more than one type of antibody, each can be directed against a different BoNT epitope.

In some cases, the compositions of the present disclosure comprise at least one or more, two or more, three or more, four or more, or all of the antibodies disclosed herein. In some cases, the composition additionally comprises a pharmaceutical carrier.

The subject composition encompasses compositions that specifically bind to one or more epitopes. The composition may also contain any first combination of antibodies described above that specifically bind to one epitope together with a second combination of antibodies that specifically binds to a different epitope. The subject composition may contain multiple combinations such that that composition may bind two, three, or more epitopes.

A composition that specifically binds to multiple epitopes may include any of the combinations described above or one or more of the antibodies disclosed in Tables 1-6.

Where combinations of antibodies are disclosed herein, such combinations can be provided in a single formulation or can be provided as separate formulations in a kit, where the separate formulations may contain a single antibody or more antibodies. Such separate formulations of a kit may be combined prior to administration or administered by separate injection.

The anti-BoNT/G antibodies provided by the present disclosure are useful for parenteral, topical, oral, or local administration, such as by aerosol or transdermally, for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges. The antibodies present in the pharmaceutical compositions of the present disclosure, when administered orally, can be protected from digestion. This is typically accomplished either by complexing the antibodies with a composition to render them resistant to acidic and enzymatic hydrolysis or by packaging the antibodies in an appropriately resistant carrier such as a liposome. Means of protecting proteins from digestion are well known in the art.

The present disclosure provides a pharmaceutical composition comprising an antibody comprising a V_{H} of a subject antibody. The present disclosure provides a pharmaceutical composition comprising an antibody comprising a V_{L} of a subject antibody. The present disclosure provides a pharmaceutical composition comprising an amino acid sequence of a V_{H} and a V_{L} of a subject antibody. In some instances, a subject pharmaceutical composition comprises an amino acid sequence comprising a V_{H} CDR1, CDR2, and CDR3 of a subject antibody and/or a V_{L} CDR1, CDR2, and CDR3 of a subject antibody.

The pharmaceutical compositions of the present disclosure are particularly useful for parenteral administration, such as intravenous administration or administration into a body cavity or lumen of an organ. The compositions for administration can comprise a solution of one or more anti-BoNT antibody dissolved in a pharmaceutically acceptable carrier, which may be an aqueous carrier. A variety of aqueous carriers can be used, e.g., buffered saline and the like.

The pharmaceutical composition of the present disclosure, in some cases, comprises: a pharmaceutically acceptable carrier; and at least a first antibody that specifically binds to Botulinum neurotoxin serotype BoNT/G, wherein the antibody specifically binds an epitope of a Botulinum neurotoxin that is specifically bound by an antibody comprising a VH comprising a CDR1, CDR2 and CDR3 and a V_{L} comprising a CDR1, CDR2 and CDR3 of the the Hu6G6.2 antibody, the Hu6G7.2 antibody, the Hu6G9.1 antibody, the Hu6G10 antibody, or the Hu6G11.2 antibody.

In some cases, the pharmaceutical composition comprises a first anti-BoNT antibody that binds a first epitope of Botulinum neurotoxin G. In some cases, the pharmaceutical composition comprises a second anti-BoNT antibody that binds a second epitope of BoNT/G. In some cases, the BoNT antibody binds more than one epitope of Botulinum neurotoxin G.

In some cases, the pharmaceutical composition comprises at least one or more, two or more, three or more, four or more, or all of the antibodies disclosed herein.

In some cases, the pharmaceutical composition additionally comprises a pharmaceutical carrier.

Non-aqueous pharmaceutically acceptable carriers (excipients) are known to those of skill in the art. Such excipients can comprise any substance that is biocompatible and liquid or soft enough at the subject's body temperature to release the active agent(s) (e.g., Anti-BoNT antibodies) into the subject's bloodstream at a desired rate. Non-aqueous carriers are usually hydrophobic and commonly organic, e.g., an oil or fat of vegetable, animal, mineral or synthetic origin or derivation. The carrier may include at least one chemical moiety of the kind that typifies “fatty” compounds, e.g., fatty acids, alcohols, esters, etc., i.e., a hydrocarbon chain, an ester linkage, or both. “Fatty” acids in this context include, but are not limited to, acetic, propionic and butyric acids through straight- or branched-chain organic acids containing up to 30 or more carbon atoms. The non-aqueous carrier may be immiscible in water and/or soluble in the substances commonly known as fat solvents. The non-aqueous carrier can correspond to a reaction product of a “fatty” compound or compounds with a hydroxy compound, e, g., a mono-hydric, di-hydric, trihydric or other polyhydric alcohol, e.g., glycerol, propanediol, lauryl alcohol, polyethylene or -propylene glycol, etc. These compounds include, but are not limited to, the fat-soluble vitamins, e.g., tocopherols and their esters, e.g., acetates sometimes produced to stabilize tocopherols. Sometimes, for economic reasons, the carrier can comprise a natural, unmodified vegetable oil such as sesame oil, soybean oil, peanut oil, palm oil, or an unmodified fat. Alternatively, the vegetable oil or fat may be modified by hydrogenation or other chemical means which is compatible with the present disclosure. The appropriate use of hydrophobic substances prepared by synthetic means is also envisioned. Non-aqueous excipient compositions can also comprise, in addition to a biocompatible oil, an “antihydration agent” which term as used herein means a substance that retards hydration of the active agent(s) and/or the biocompatible oil or fat and thereby further decreases and/or stabilizes the rate of release of the active agent(s) from that composition following administration to an animal (e.g., human). A great variety of non-toxic antihydration agents are known. By way of example there are “gelling” agents that, when dispersed, and in some cases heated to dissolve them in the oil, give the body of oil greater visco-elasticity (and therefore greater structural stability) and thereby slow down penetration of the oil by body fluids.

Illustrative antihydration agents include various polyvalent metal salts or complexes of organic acids, for instance fatty acids having from about 8 or 10 to about 20 or 22 carbon atoms, e.g. aluminum, zinc, magnesium or calcium salts of lauric acid, palmitic acid, stearic acid and the like. Such salts can be mono-, di- or tri-substituted, depending on the valence of the metal and the degree of oxidation of the metal by the acid. Of common usage are the aluminum salts of such fatty acids. Aluminum monostearate and distearate are frequently used anti-hydration agents. Others that are useful include aluminum tristearate, calcium mono- and distearate, magnesium mono- and distearate and the corresponding palmitates, laurates and the like. The concentration of such an antihydration agent, based on the weight of the oil plus that agent, may be between about 1% and about 10% (most typically between about 2% and about 5%), although other concentrations may be suitable in some cases.

The various solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of anti-BoNT/G antibody in these formulations can vary widely, and will be selected primarily based on fluid volumes, viscosities, body weight and the like in accordance with the particular mode of administration selected and the patient's needs. In some instances, the solutions may be stored in lyophilized or frozen form. Examples of suitable anti-BoNT antibody formulations are described in WO 2011/028961.

Thus, a typical pharmaceutical composition for intravenous administration would be about 0.1 mg to 10 mg per patient per day. Dosages from about 1 mg up to about 200 mg per patient per day can be used. Methods for preparing parenterally administrable compositions will be known or apparent to those skilled in the art and are described in more detail in such publications as Remington's Pharmaceutical Science, 15th ed., Mack Publishing Company, Easton, Pennsylvania (1980).

The compositions containing the anti-BoNT/G antibodies of the present disclosure or a cocktail thereof can be administered for therapeutic and/or prophylactic treatments. Pharmaceutical compositions can be administered in a dosage sufficient to neutralize (mitigate or eliminate) the BoNT toxin(s) (i.e., reduce or eliminate a symptom of BoNT poisoning (botulism)). An amount adequate to accomplish this is defined as a “therapeutically effective dose.” Amounts effective for this use will depend upon the severity of the disease and the general state of the patient's health.

Single or multiple administrations of the compositions may be administered depending on the dosage and frequency as required and tolerated by the patient. In any event, the composition should provide a sufficient quantity of the antibodies of the present disclosure to effectively treat the patient.

The present disclosure thus provides a method of specifically binding to, and in some cases, neutralizing a Botulinum neurotoxin in an individual (e.g., a human; or a non-human mammal), the method generally involving administering to the individual an effective amount of a subject anti-BoNT antibody, or an effective amount of a subject composition comprising a subject anti-BoNT antibody. The treatments essentially comprise administering to the poisoned organism (e.g., human or non-human mammal) a quantity of one or more neutralizing antibodies sufficient to neutralize (e.g., mitigate or eliminate) symptoms of BoNT/G poisoning. Administering the antibody, or the composition comprising the antibody, provides for specific binding to and, in some embodiment, neutralization of Botulinum neurotoxin present in the individual. The BoNT/G poisoning can be due to ingestion of contaminated food products (food botulism), can result from an anaerobic wound infection (wound botulism), or can result from an act of biological warfare or bioterrorism.

The present disclosure also provides methods of reducing the likelihood that an individual at risk of exposure to Botulinum neurotoxin will experience symptoms of Botulinum neurotoxin poisoning following exposure to the Botulinum neurotoxin (e.g., where the exposure is via inhalation, via ingestion, via a wound infection, or via another route/mode of exposure). Administration of a subject antibody or subject composition reduces the likelihood that the individual will experience symptoms of Botulinum neurotoxin poisoning. Thus, e.g., a subject anti-BoNT antibody, or a subject composition comprising a subject anti-BoNT antibody, can be administered to an individual before the individual has Botulinum neurotoxin poisoning, e.g., before a BoNT is present in the individual. For example, a subject anti-BoNT antibody, or a subject composition comprising a subject anti-BoNT antibody, can be administered to an individual who is at risk of BoNT exposure, e.g., an individual who is at greater risk than the general population of experiencing Botulinum neurotoxin exposure and poisoning. Such individuals include, e.g., military personnel, e.g., military personnel in a combat setting; personnel involved in investigation or clean up of a site suspected of involving Botulinum neurotoxin exposure (e.g., hazardous materials (“hazmat”) personnel) and other individuals who are at risk of Botulinum neurotoxin exposure, either accidental or intentional.

### XI. Kits for Diagnosis or Treatment

Kits for the treatment of botulism or for the detection/confirmation of a Clostridium botulinum infection are also provided. Kits will typically comprise one or more anti-BoNT/G antibodies (e.g., anti-BoNT antibodies in a composition for pharmaceutical use). For diagnostic purposes, the antibody(s) can optionally be labeled. In addition, the kits will typically include instructional materials disclosing means of use anti-BoNT antibodies in the treatment of symptoms of botulism. The kits may also include additional components to facilitate the particular application for which the kit is designed. Thus, for example, where a kit contains one or more anti-BoNT antibodies for detection of diagnosis of BoNT subtype, the antibody can be labeled, and the kit can additionally contain means of detecting the label (e.g., enzyme substrates for enzymatic labels, filter sets to detect fluorescent labels, appropriate secondary labels such as a sheep anti-human antibody, or the like). The kits may additionally include buffers and other reagents routinely used for the practice of a particular method. Such kits and appropriate contents are well known to those of skill in the art.

Kits provided for the treatment of botulism may contain one or more anti-BoNT/G antibodies. The antibodies can be provided separately or mixed together. Typically, the antibodies will be provided in a sterile pharmacologically acceptable excipient. The antibodies can also be provided pre-loaded into a delivery device (e.g., a disposable syringe).

The kits can optionally include instructional materials teaching the use of the antibodies, recommended dosages, contraindications, and the like.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-33 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. An isolated antibody that specifically binds to a Botulinum neurotoxin (BoNT), wherein the antibody competes for binding to BoNT with an antibody comprising:
a) a variable heavy (V_{H}) chain comprising heavy complementarity determining regions 1-3 (HCDRs 1-3) and a variable light (V_{L}) chain comprising light CDRs 1-3 (LCDRs 1-3) of a pair of V_{H} chain and V_{L} chain of an antibody listed in Table 1, Table 2, Table 3, Table 4, Table 5, or Table 6; or
b) a V_{H} chain comprising the amino acid sequence of the V_{H} chain and a V_{L} chain comprising the amino acid sequence of a pair of V_{H} chain and V_{L} chain of an antibody listed in Table 1, Table 2, Table 3, Table 4, Table 5, or Table 6.
2. The isolated antibody of aspect 1, wherein the isolated antibody competes for binding to BoNT with an antibody comprising:
a V_{H} chain comprising HCDRs 1-3 and a V_{L} chain comprising LCDRs 1-3 of a pair of V_{H} chain and V_{L} chain of an antibody listed in Table 1, Table 2, Table 3, Table 4, Table 5, or Table 6.
3. The isolated antibody of aspect 2, wherein the isolated antibody competes for binding to BoNT with an antibody comprising:
a V_{H} chain comprising HCDRs 1-3 and a V_{L} chain comprising LCDRs 1-3 of a pair of V_{H} chain and V_{L} chain of the Hu6G6.2 antibody, the Hu6G7.2 antibody, the Hu6G9.1 antibody, the Hu6G10 antibody, or the Hu6G11.2 antibody.
4. The isolated antibody of aspect 3, wherein the isolated antibody competes for binding to BoNT with an antibody comprising:
a V_{H} chain comprising HCDRs 1-3 and heavy chain framework regions 1-4 (H-FRs 1-4) and a V_{L} chain comprising LCDRs1-3 and light chain framework regions 1-4 (L-FRs 1-4) of the Hu6G6.2 antibody, the Hu6G7.2 antibody, the Hu6G9.1 antibody, the Hu6G10 antibody, or the Hu6G11.2 antibody.
5. The isolated antibody of aspect 1, wherein the isolated antibody competes for binding to BoNT with an antibody comprising:
a V_{H} chain comprising the amino acid sequence of the V_{H} chain and a V_{L} chain comprising the amino acid sequence of a pair of V_{H} chain and V_{L} chain of an antibody listed in Table 1, Table 2, Table 3, Table 4, Table 5, or Table 6.
6. The isolated antibody of aspect 5, wherein the isolated antibody competes for binding to BoNT with an antibody comprising:
a V_{H} chain comprising the amino acid sequence of the V_{H} chain and a V_{L} chain comprising the amino acid sequence of the V_{L} chain of a pair of V_{H} chain and V_{L} chain of the Hu6G6.2 antibody, the Hu6G7.2 antibody, the Hu6G9.1 antibody, the Hu6G10 antibody, or the Hu6G11.2 antibody.
7. The isolated antibody of aspect 6, wherein the isolated antibody competes for binding to BoNT with an antibody comprising:
at least 80%, at least 90%, or at least 95% sequence identity to a V_{H} chain comprising the amino acid sequence of the V_{H} chain and a V_{L} chain comprising the amino acid sequence of the V_{L} chain of a pair of V_{H} chain and V_{L} chain of the Hu6G6.2 antibody, the Hu6G7.2 antibody, the Hu6G9.1 antibody, the Hu6G10 antibody, or the Hu6G11.2 antibody.
8. An isolated antibody that binds to a Botulinum neurotoxin (BoNT), wherein the antibody comprises:
   a) a variable heavy (VII) chain comprising heavy complementarity determining regions 1-3 (HCDRs 1-3) and a variable light (V_{L}) chain comprising light CDRs 1-3 (LCDRs 1-3) of a pair of VH chain and V_{L} chain of an antibody listed in Table 1, Table 2, Table 3, Table 4, Table 5, or Table 6;
   b) a V_{H} chain comprising the amino acid sequence of the V_{H} chain and a V_{L} chain comprising the amino acid sequence of the V_{L} chain of a pair of V_{H} chain and V_{L} chain of an antibody listed in Table 1, Table 2, Table 3, Table 4, Table 5, or Table 6;
   c) a V_{H} chain comprising the amino acid sequence of the V_{H} chain of an antibody listed in Table 1, Table 2, Table 3, Table 4, Table 5, or Table 6;
   d) a V_{L} chain comprising the amino acid sequence of the V_{L} chain of an antibody listed in Table 1, Table 2, Table 3, Table 4, Table 5, or Table 6;
   e) a V_{H} chain comprising HCDRs 1-3 of an antibody listed in Table 1, Table 2, Table 3, Table 4, Table 5, or Table 6; or
   f) a V_{L} chain comprising LCDRs 1-3 of an antibody listed in Table 1, Table 2, Table 3, Table 4, Table 5, or Table 6.
9. The isolated antibody of aspect 8, wherein the antibody comprises:
   a V_{H} chain comprising HCDRs 1-3 and a V_{L} chain comprising LCDRs 1-3 of a pair of V_{H} chain and V_{L} chain of an antibody listed in Table 1, Table 2, Table 3, Table 4, Table 5, or Table 6.
10. The isolated antibody of aspect 9, wherein the antibody comprises:
   a V_{H} chain comprising the amino acid sequence of the V_{H} chain and a V_{L} chain comprising the amino acid sequence of the V_{L} chain of a pair of V_{H} chain and V_{L} chain of an antibody listed in Table 1, Table 2, Table 3, Table 4, Table 5, or Table 6.
11. The isolated antibody of aspect 9, wherein the antibody comprises:
a V_{H} chain comprising an amino acid sequence having at least 80%, at least 90%, or at least 95% sequence identity to a V_{H} chain and a V_{L} chain comprising an amino acid sequence having at least 80%, at least 90%, or at least 95% sequence identity to a V_{L} chain of the Hu6G6.2 antibody, the Hu6G7.2 antibody, the Hu6G9.1 antibody, the Hu6G10 antibody, or the Hu6G11.2 antibody.
12. The isolated antibody of aspect 9, wherein the antibody comprises:
a V_{H} chain and a V_{L} chain of the Hu6G6.2 antibody, the Hu6G7.2 antibody, the Hu6G9.1 antibody, the Hu6G10 antibody, or the Hu6G11.2 antibody.
13. The isolated antibody of any one of aspects 1, 2, 5, or 8-10, wherein the antibody is a human antibody or a humanized antibody.
14. The isolated antibody of any one of aspects 1-13, wherein the antibody is a single chain Fv (scFv), IgG, Fab, (Fab′)₂, or (scFv′)₂.
15. The isolated antibody of any one of aspects 1-14, wherein the antibody binds to BoNT/G with a Kp of about 5 nM or less.
16. A composition comprising:
   a pharmaceutically acceptable carrier; and
   at least a first antibody according to any one of aspects 1-15.
17. The composition of aspect 16, comprising a first antibody and a second antibody according to any one of aspects 1-15.
18. The composition of aspect 17, wherein the first antibody binds to a first epitope of BoNT/G and the second antibody binds to a second epitope of BoNT/G.
19. An isolated nucleic acid comprising a nucleotide sequence encoding the amino acid sequence of any one of the antibodies of any one of aspects 1-15.
20. A vector comprising the isolated nucleic acid of aspect 19.
21. A first nucleic acid comprising a nucleotide sequence encoding the amino acid sequence of the V_{H} chain and a second nucleic acid comprising a nucleotide sequence encoding the amino acid sequence of the V_{L} chain any one of the antibodies of any one of aspects 1-15.
22. A first vector comprising the first nucleic acid and a second vector comprising the second nucleic acid of aspect 21.
23. A host cell comprising the nucleic acid of aspect 19, the vector of aspect 20, the first and second nucleic acids of aspect 21 or the first and second vectors of aspect 22.
24. The isolated antibody according to any one of aspects 1-15, wherein the antibody is labeled.
25. A method of specifically binding to a Botulinum neurotoxin in a mammal comprising:
   administering to the mammal an effective amount of an antibody of any one of aspects 1-15;
   wherein the administering provides for specific binding of Botulinum neurotoxin present in the mammal.
26. A method of specifically binding to a Botulinum neurotoxin in a mammal, comprising:
   administering to the mammal an effective amount of a composition of any one of aspects 16-18;
   wherein the administering provides for specific binding of Botulinum neurotoxin present in the mammal.
27. A method for detecting Botulinum neurotoxin in a sample, comprising:
   contacting an antibody of any one of aspects 1-15 with the sample,
   detecting binding of said antibody to Botulinum neurotoxin in the sample.
28. The method of aspect 27, wherein the antibody is labeled.
29. The method of aspect 28, wherein said label is a fluorescent label, a chemiluminescent label, a radiolabel, an enzyme, or a chromogenic label.
30. The method of any one of aspects 27-29, wherein said sample is blood.
31. The method of any one of aspects 27-30, wherein the antibody is immobilized on a substrate.
32. The method of any one of aspects 27-31, wherein the antibody is conjugated to a nucleic acid and wherein said detecting comprises a polymerase chain reaction.
33. A method of reducing the likelihood that an individual at risk of exposure to Botulinum neurotoxin will experience symptoms of Botulinum neurotoxin poisoning following exposure, the method comprising administering to said individual an effective amount of an antibody of any one of aspects 1-15;
   wherein said administering provides for reducing the likelihood that the individual will experience symptoms of Botulinum neurotoxin poisoning.

### EXAMPLES

The following examples are offered to illustrate, but not to limit any embodiments provided by the present disclosure.

### Example 1

### Confirmation of mAb Binding to BoNT/G Holotoxin and BoNT/G Domains

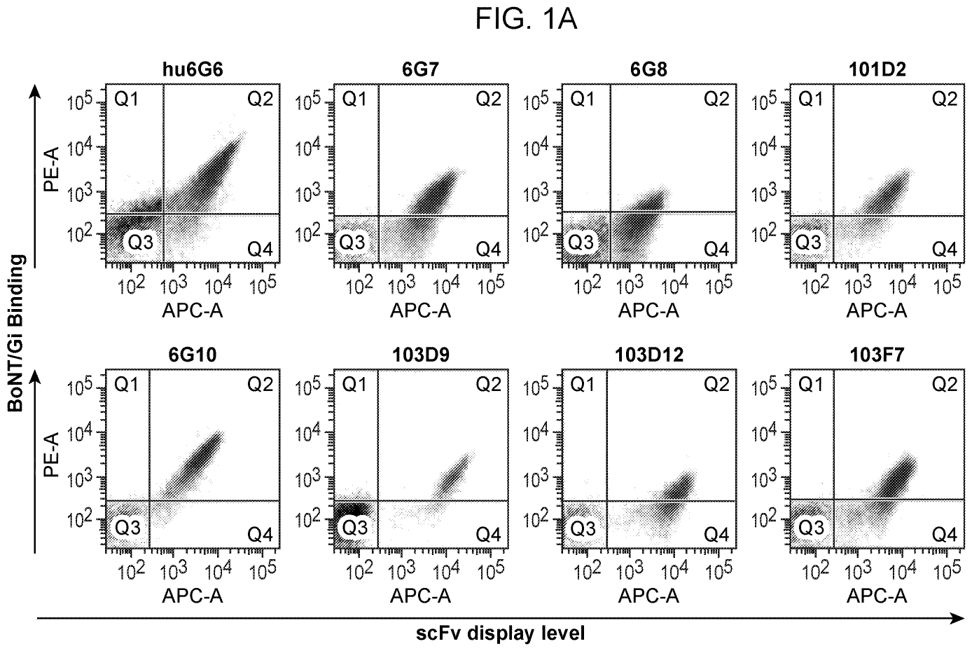

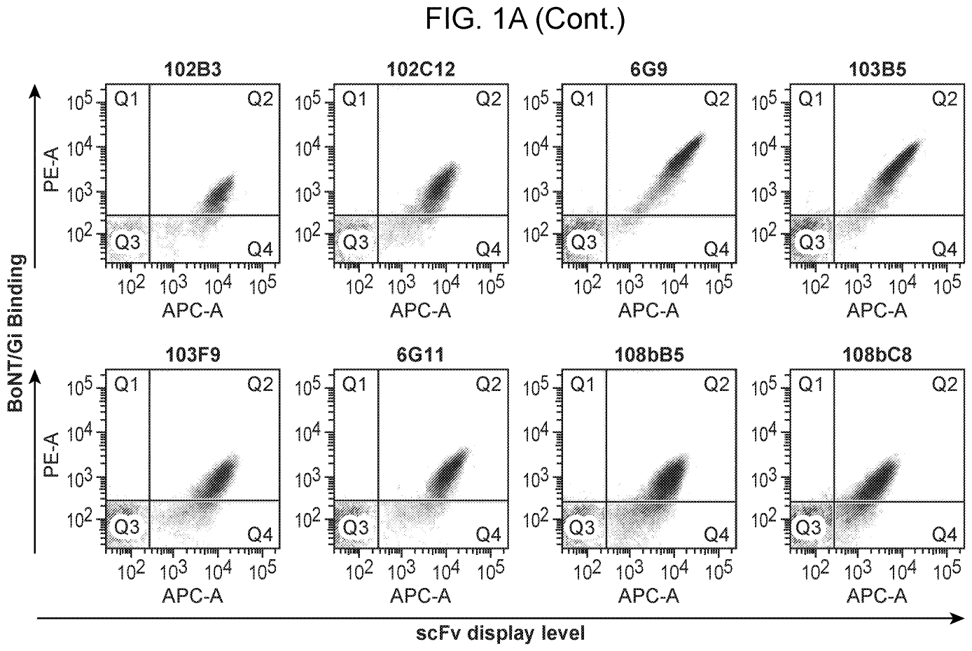

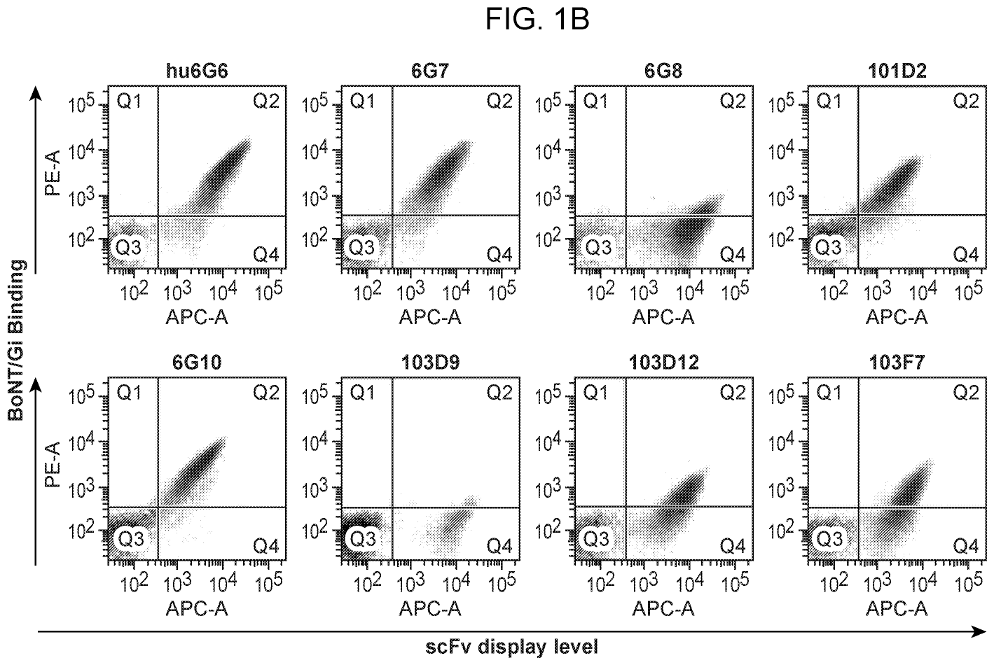

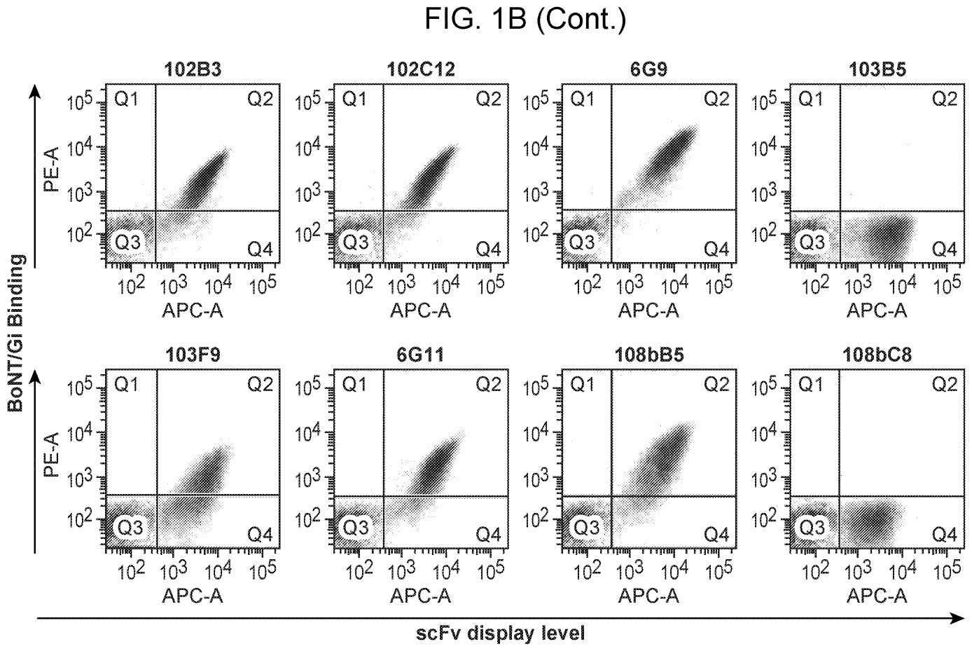

The crude BoNT/G holotoxin from Metabiologics (1:25 dilution) was used for confirmation of the mAb binding side by side with 50 nM of BoNT/Gi binding. The mouse serum from BoNT/Gi immunized mice (1:500 dilution) and equine anti-BoNT/G serum (1:2000 dilution) was used for detection. As shown in FIG. 1 (x-axis for each plot indicates increasing scFv display level, y-axis for each plot indicates increasing BoNT/G or Gi binding), yeast displayed scFv mAbs were incubated with 50 nM inactive BoNT/G (BoNT/Gi, FIG. 1A) or 1:25 active crude BoNT/G for 1 hour at room temperature. Mouse serum immunized with BoNT/Gi (1:500) followed with PE-conjugated goat-anti mouse IgG (1 ug/ml) and Alexa-647 conjugated anti-SV5 IgG (1:1000) was used for detection, tested with a flow cytometer. FIG. 1A showed that all the 16 mAbs bound BoNT/Gi, while FIG. 1B showed that 14 mAbs bound active BoNT/G. The mAbs 103B5 and 108bC8 bound inactive BoNT/Gi but not bind active BoNT/G.

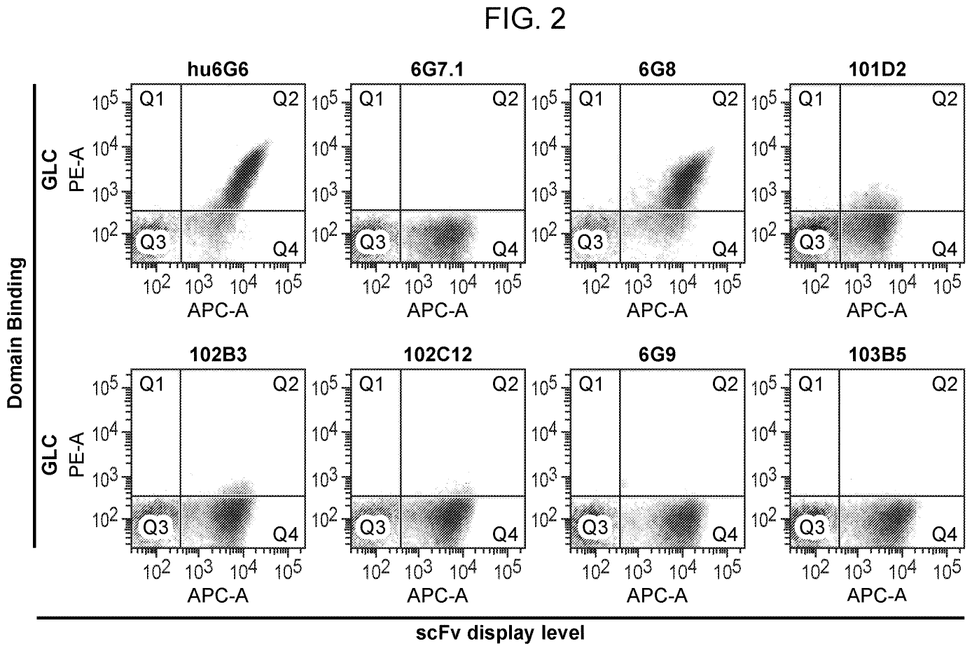

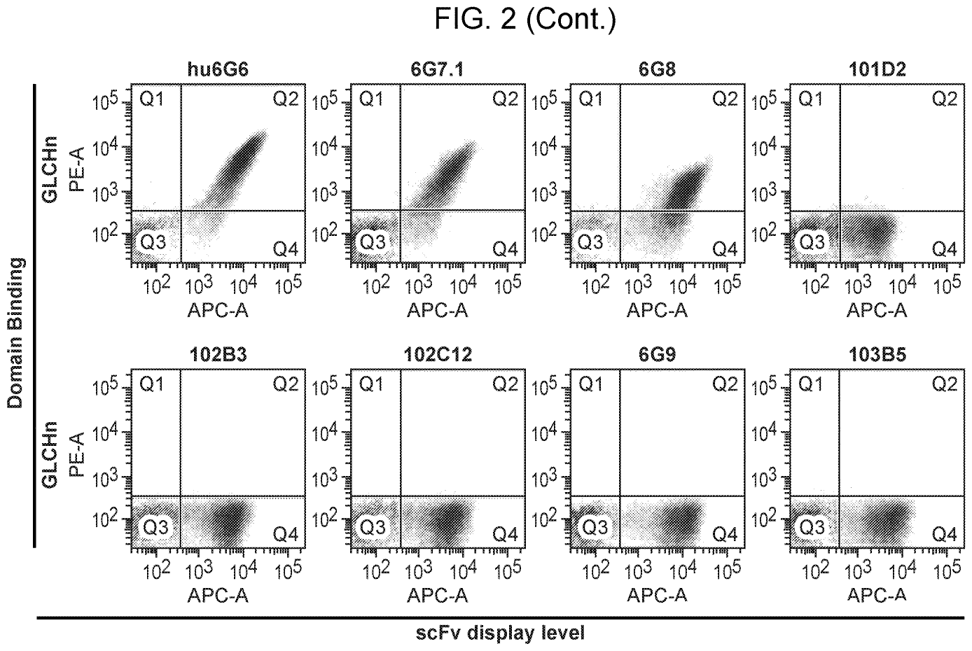

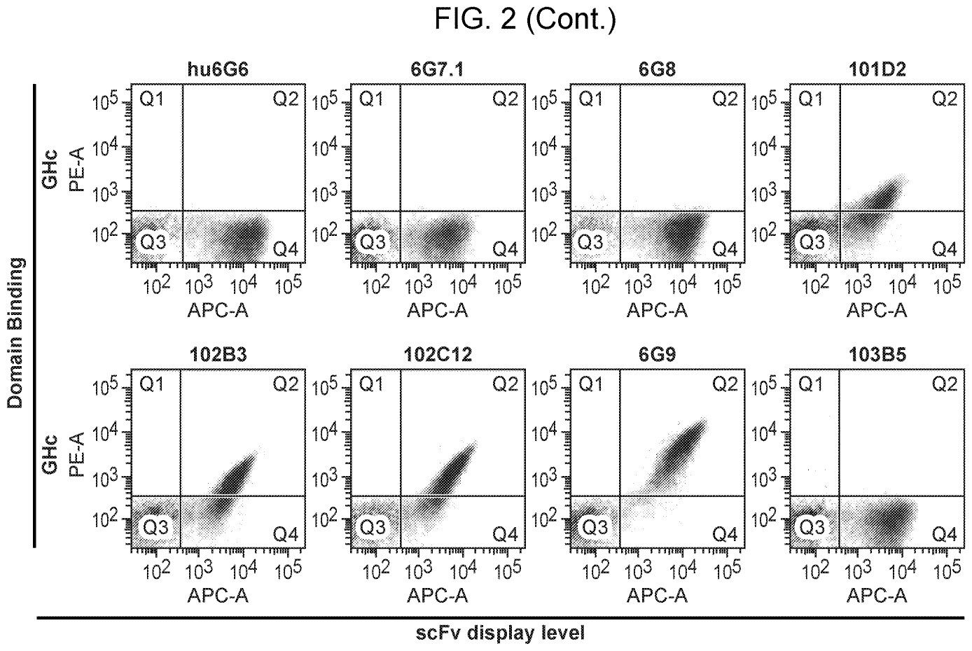

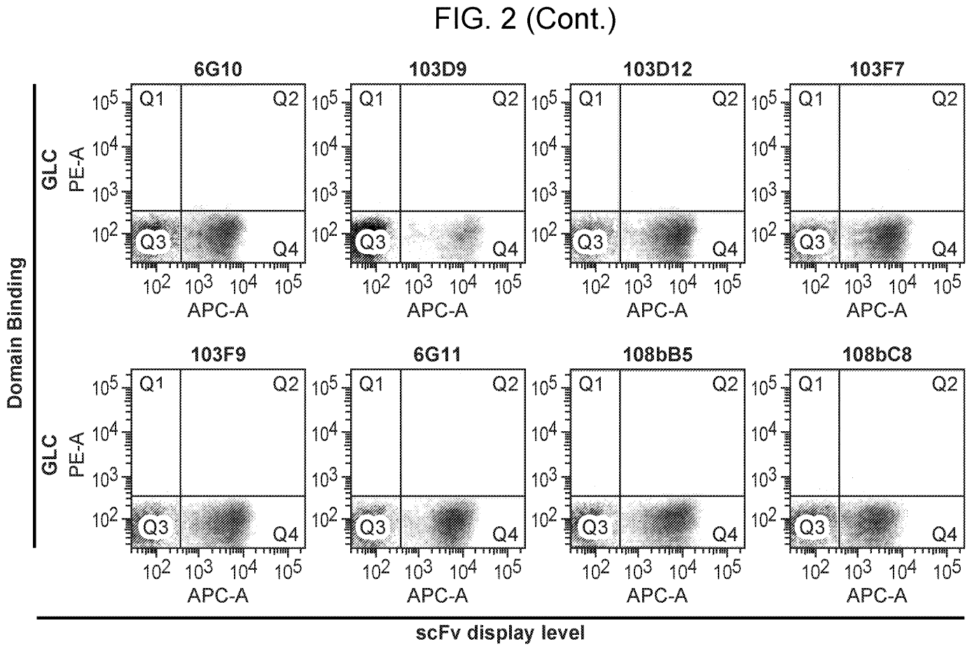

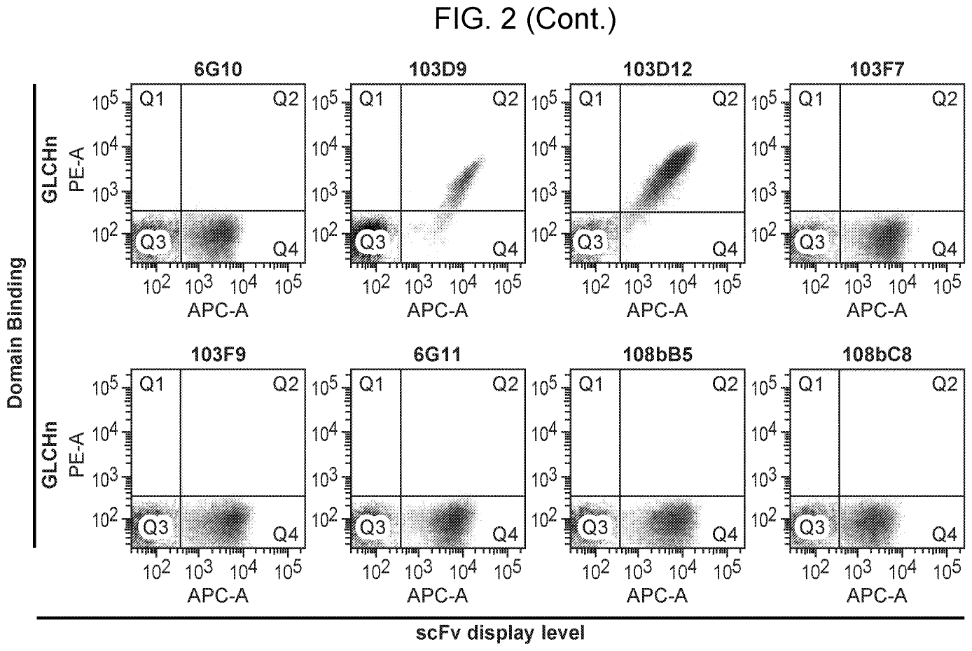

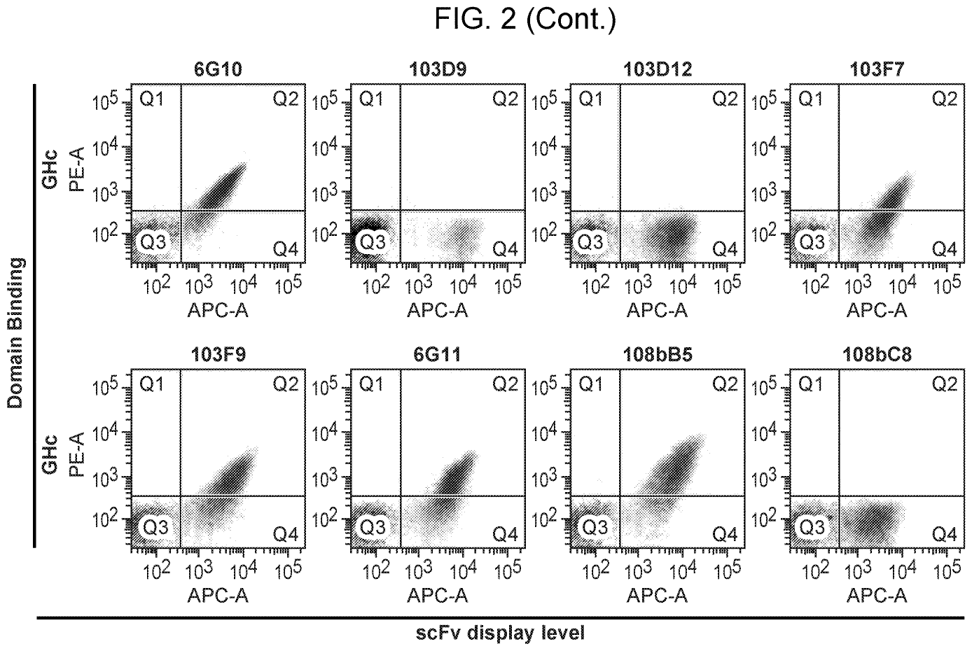

Recombinant BoNT/G LC, G LCH_{N}, G Hc-MBP were used to identify the binding domain of the mAbs on BoNT/G surface. As shown in FIG. 2 (x-axis for each plot indicates increasing scFv display level, y-axis for each plot indicates increasing BoNT/G or Gi domain binding), yeast displayed scFv mAbs were incubated with 100 nM BoNT/G LC, LCHN or Hc-MBP for 1 hour at room temperature. Mouse serum immunized with BoNT/Gi (1:500) followed with PE-conjugated goat-anti mouse IgG (1 μg/ml) and Alexa-647 conjugated anti-SV5 IgG (1:1000) was used for detection, tested with a flow cytometer.

The results showed that the mAbs bound on different BoNT/G domain. Two mAbs, 6G6 and 6G8, bound BoNT/GLC. Three mAbs 6G7, 103D9 and 103D12 bound GLC-Hy but not bind GLC, based on which it was deduced that they could bind on the domain GHN. Nine mAbs, 6G9, 6G10, 101D2, 102B3, 102C12, 103F7, 103F9, 6G11 and 108Bb5 bound BoNT/G Hc. The two mAbs 103B5 and 108bC8 that bound BoNT/Gi but not bind BoNT/G active toxin did not show binding to any of the three domains (FIG. 2). The experiment was repeated with equine anti-BoNT/G serum (1:2000 dilution) for detection, showing the same results (data not shown).

Further testing was performed on the colonies 108aF7, 108aG2 and 108bE8 that had similar VH-CDR3 with 103B5 but had several site mutations with inactive 50 nM BoNT/Gi, crude 1:25 dilution of active BoNT/G and 100 nM of different BoNT/G domains. The results confirmed that either 103B5 or the three 103B5-related colonies only bound BoNT/Gi, but did not bind active BoNT/G or any of the domains. Failure of the scFvs to bind the active BoNT/G holotoxin or domains could result from the possibility that the scFvs might bind epitopes existing on BoNT/Gi only but not on active BoNT/G or domains. Based on the data it could be concluded that there might be slight difference in spatial structure among the active BoNT/G, inactive BoNT/Gi and the BoNT/G domains.

### Example 2

### mAb Overlap Analysis and Clustering

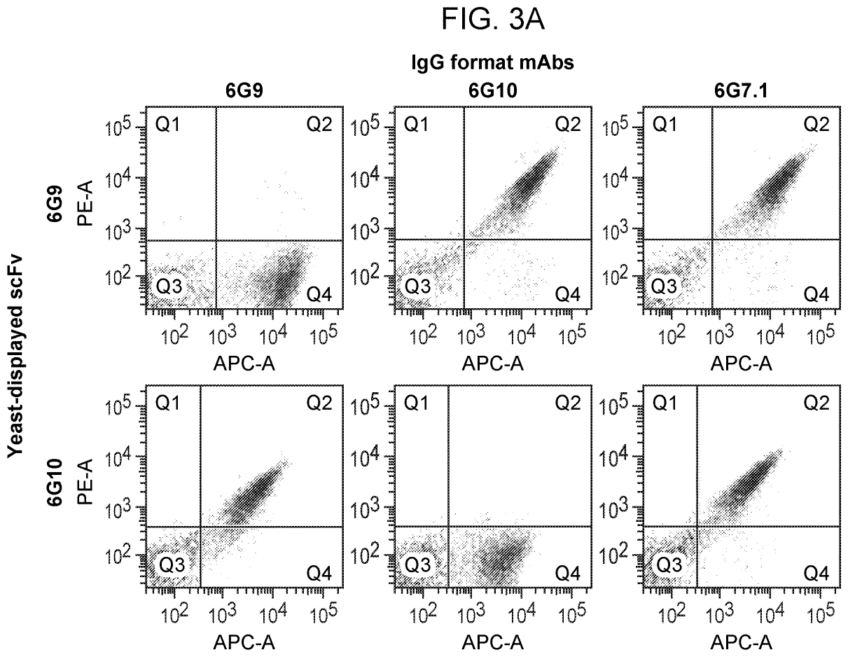

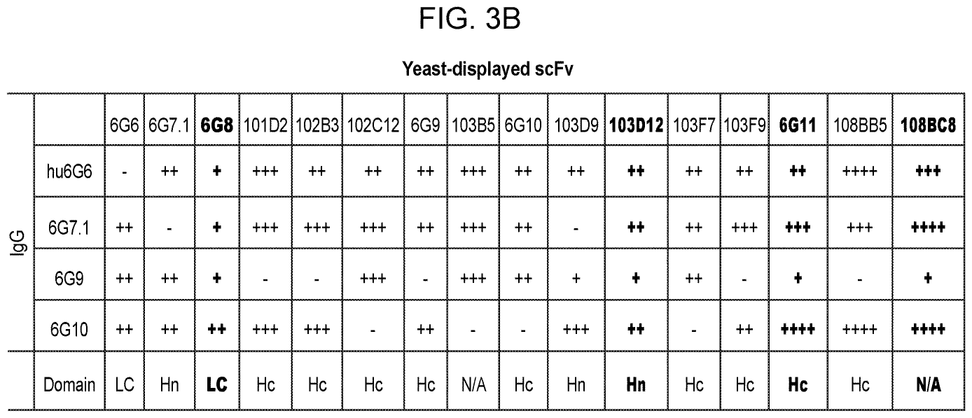

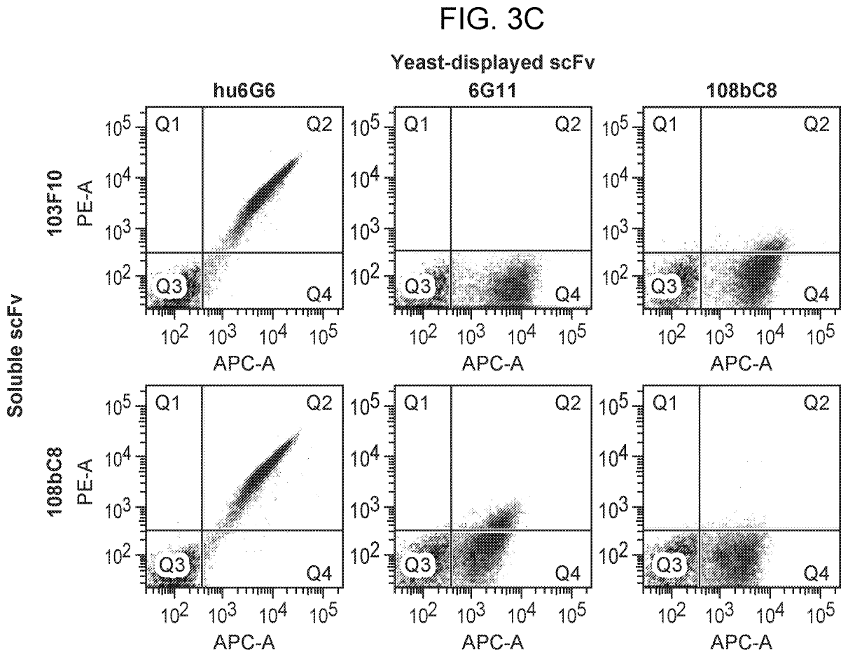

IgG was prepared for the four mAbs hu6G6 (humanized 6G6), 6G7.1 (affinity maturated 6G7, described below). As shown in FIG. 3, yeast-displayed scFv was incubated with 50 nM BoNT/Gi, detected with IgG (6G6, 6G7.1, 6G9 or 6G10 in panel A and B) plus PE-conjugated goat-antihuman IgG, or soluble scFv (6G11 or 108bC8 in panel C) plus 9E10 IgG with PE-conjugated goat-anti mouse IgG. FIG. 3A shows 6G9 (6G10) did affect 6G10 (or 6G9) binding of BoNT/Gi. Detection with 6G7.1 acted as a positive control. FIG. 3B showed that all the mAbs had separate epitopes and did not compete with 6G6, while 103D9 competed with 6G7.1, 101D2, 102B3 and 103F9 competed with 6G9, and 102C12, 103B5 and 103F7 competed with 6G10. Four mAbs, 6G8, 103D12, 6G11 or 108C8, were not competed by all the four IgGs (− indicates no binding signal was detected, suggesting the competition of the two antibodies; +, ++, +++ or ++++, indicates binding signal was detected suggesting no competition of the two antibodies). FIG. 3C shows that 6G11 and 108bC8 did not compete with each other fully. Detection with hu6G6 served as a positive control.

Since 6G7 was isolated with BoNT/Gi followed by 6G6 detection, they had non-overlapped epitope. In addition, competitive analysis of 6G8 and 6G9 showed that they did not affect each other in BoNT/Gi binding, which suggests they also had unique epitope, leading to the conclusion that the four mAbs had four separate epitopes on BoNT/G. Then the mAbs were clustered by binding competition with the four mAbs. The results showed that all the mAbs had separate epitope from 6G6, while 103D9 shared epitope with 6G7.1, 101D2, 102B3 and 103F9 shared epitope with 6G9, and 102C12, 103B5 and 103F7 shared epitopes with 6G10. Since 103G5 that only bound BoNT/Gi overlapped with 6G10, it was deduced that it also bound on Hc domain of BoNT/Gi. Four mAbs 6G8, 103D12, 6G11 or 108C8 were not competed by any of the four IgGs. As is known that 6G8 bound GLC and 103D12 bound LCHN but not bind LC, they would have unique epitope. Soluble scFv was then prepared for the two Hc-binding mAbs 6G11 and 108bC8 for competition analysis. The results showed that they did not compete completely with each other, suggesting they had unique epitope on BoNT/Gi.

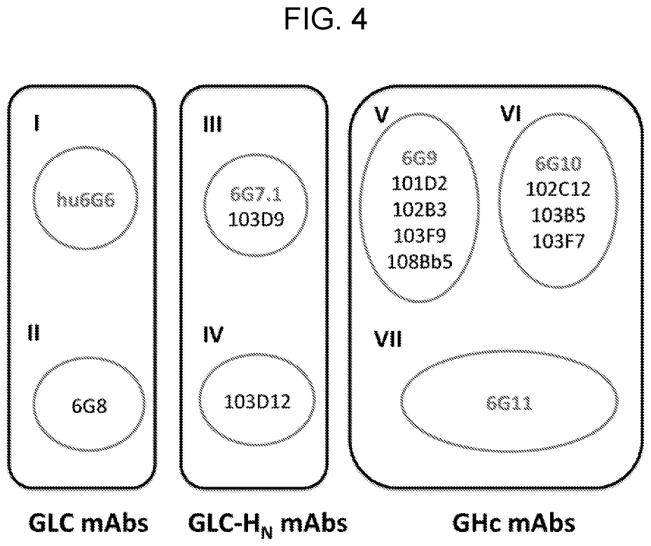

Taken together, it was concluded that the sixteen mAbs bound eight unique epitopes on BoNT/Gi, and the mAbs were clustered into 8 different groups I-VIII. As shown in FIG. 4, Yeast-displayed scFv mAbs were incubated with 50 nM BoNT/Gi, followed with soluble scFv plus 9E10 or BoNT/Gi IgG (hu6G6, 6G7.1, 6G9 or 6G10) and a PE-conjugated goat-anti mouse or human IgG was applied for detection with flow cytometry. The results showed that the sixteen mAbs bound to eight unique epitopes on the BoNT/Gi surface. MAbs in groups I-II bound on BoNT/G LC, mAbs in groups III-IV bound on LCH_{N} (most probably bound on H_{N} since they did not bind LC), while mAbs in groups V-VIII bound on BoNT/G Hc. Two mAbs (group I, hu6G6 and group II, 6G8) bound LC with separate epitopes, three mAbs bound LCHN but not LC with two epitopes (group III, 6G7.1 and 103D9; group IV, 103D12) and ten mAbs bound Hc with three non-overlapping epitopes (group V, VI and VII). One mAb 108bC8 (group VIII) had unique epitope that only existed on BoNT/Gi but the binding domain was not determined. MAbs 103B5 and 108bC8 bound BoNT/Gi but not BoNT G. 103B5 shared an epitope with 6G10 on Hc, 108bC8 had an undetermined, unique epitope on BoNT/Gi (FIG. 4).

### Example 3

### mAb Humanization and Affinity Maturation

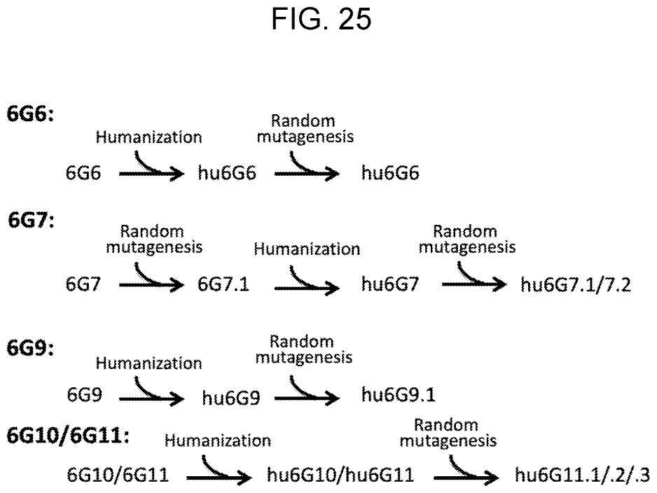

Five lead antibodies 6G6, 6G7, 6G9, 6G10 and 6G11 were humanized and affinity maturated for BoNT/Gi binding as shown in FIG. 25. For antibody humanization, one way used was this: the humanized VH gene was synthesized and cloned into a human VK light chain library to form a humanization chain-shuffling library. The library was screened through FACS sorting high stringency to isolate the humanized mAb with a fully human light chain and potentially higher affinity than the parental mouse mAb. The advantage of the method is that humanization and affinity maturation are performed simultaneously and only several critical mouse amino acids remain in the VH.

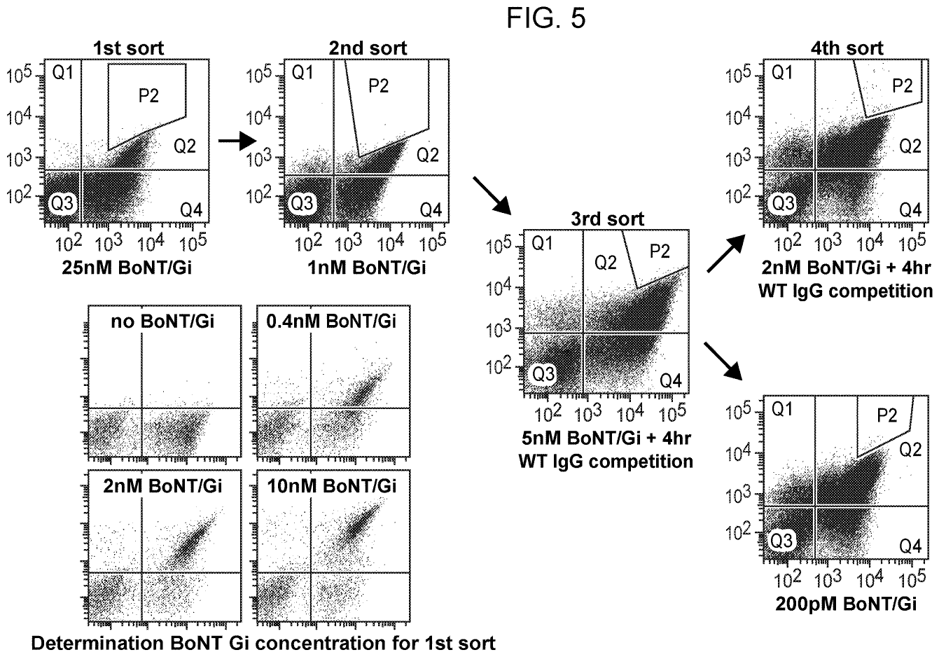

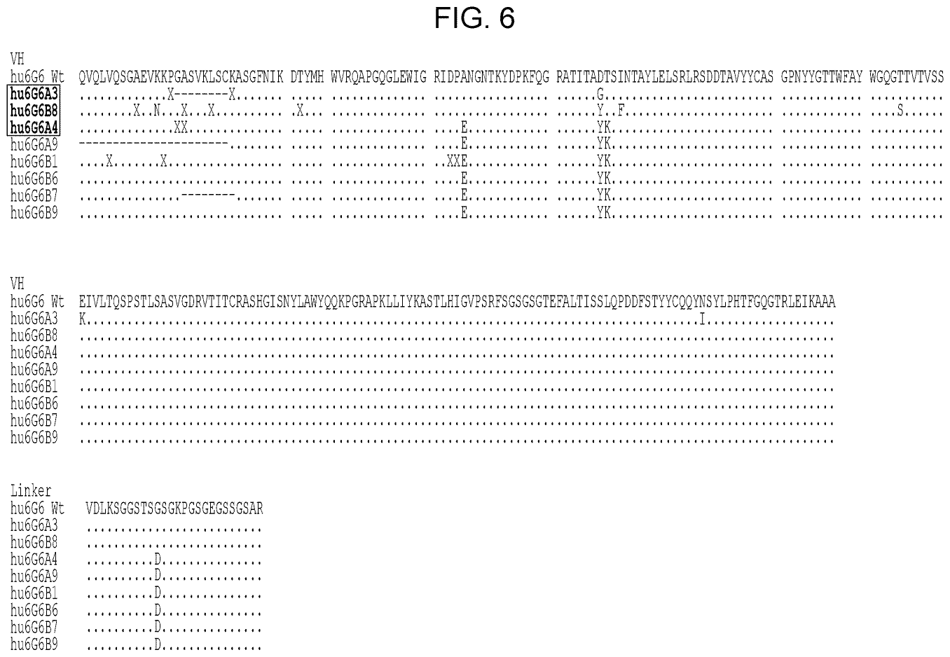

The library was first sorted with decreasing BoNT/Gi concentration, then with dissociation isolation using competition with wild type hu6G6 (FIG. 5). Colonies from the 4^{th} sort were sequenced (FIG. 6).

Sequencing of the colonies from the hu6G6 library sortout is shown in FIG. 6. Results of sequence analysis and binding measurements are shown in Table 8. Variants (A3, A4, B8) all had improved binding relative to the hu6G6 wild type. Mutations in the framework and linker regions were mutated back to the human sequences in A3.1, A4.1 and B8.1 without significant reduction in binding affinity.

Variant A4.1 was converted to IgG, named hu6G6.1 and the K_{D} was determined to be 476.45 pM.

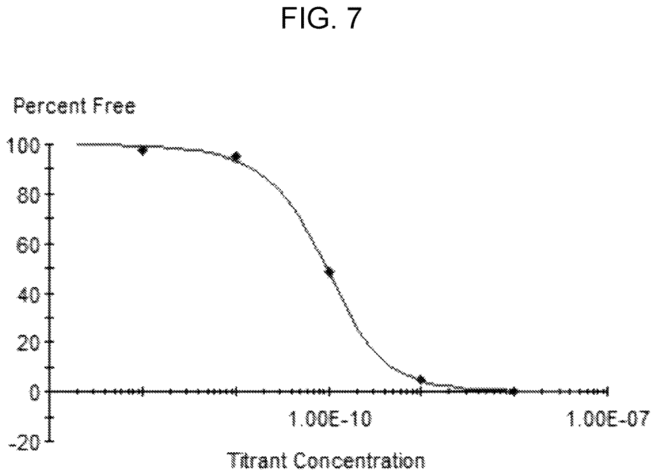

IgG format of the affinity further-matured G mAbs was produced. hu6G6.2, affinity matured from hu6G6.1 had a K_{D} value of 42.2 pM (62.86-22.45 pM, 95% confidence range) (FIG. 7).

TABLE 8
Variants of hu6G6, mutations and binding affinities as scFv on yeast surface

| | | | | A4 | | | |
|---|---|---|---|---|---|---|---|
| | | | | VH CDR2:A54E, | | B8 | |
| | | A3 | | VH Fw3:D73Y, | A4.1 | VH Fw3:K12N, | B8.1 |
| | Wt | VH Fw3:D73G; | A3.1 | T74K | VH Fw3:A54E, | D73Y, I76F; | VH Fw3:K12N, |
| Mutations | — | VK Fw1:E1K | VH Fw3:D73G | Linker:G to D | D73Y, T74K | VH Fw4 T115S | D73Y, I76F |
| Binding Max | 3150 | 6259 | 5884 | 8639 | 5981 | 6612 | 4422 |
| @25 nM | | | | | | | |
| Measured | 23.2 ± 1.89 | 5.18 ± 0.36 | 6.5 ± 0.46 | 3.46 ± 0.42 | 4.27 ± 0.48 | 3.75 ± 0.45 | 5.43 ± 0.07 |
| K_{D} (nM) | | | | | | | |
| Improvement | — | 4.5x | 3.5x | 6.8x | 5.5x | 6.2x | 4.3x |
| over WT | | | | | | | |

### Example 4

### 6G9 Humanization and Affinity Maturation

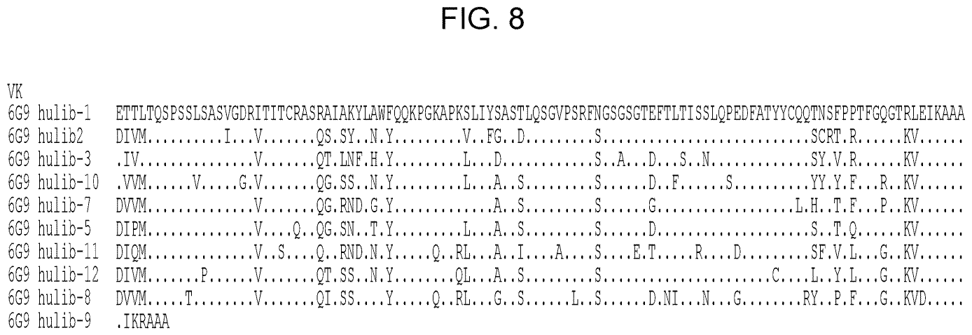

1) Humanized 6G9 VH (huVH) was cloned into a human VK library to make a chain shuffling library. The diversity of the library was determined by sequencing of 10 randomly picked colonies (FIG. 8).

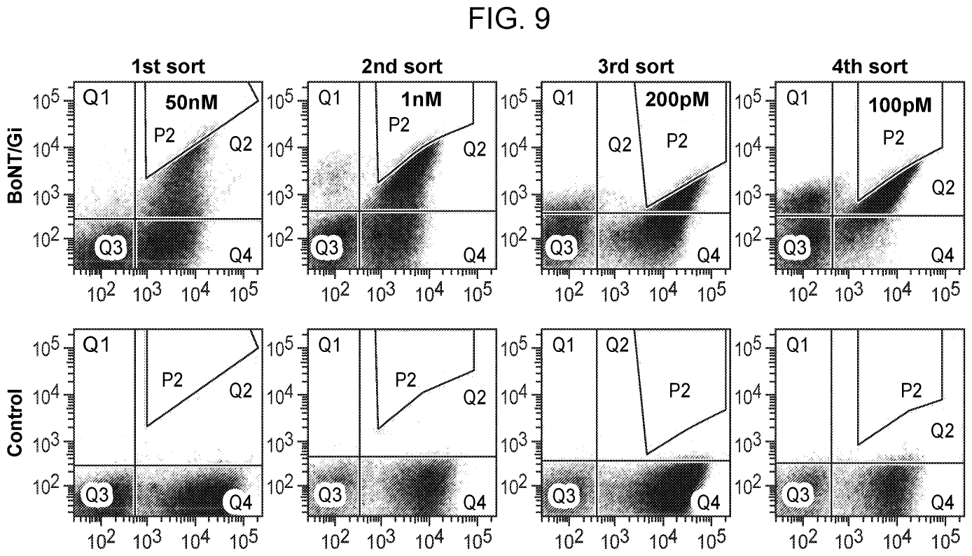

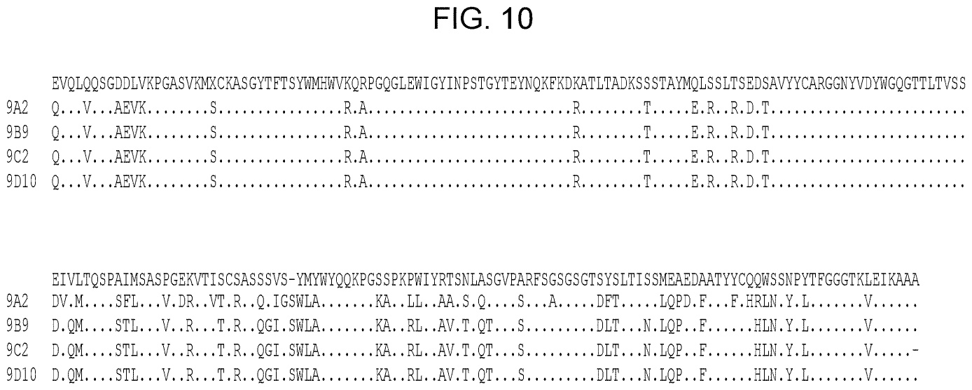

The sorting strategy and dot plots for humanization of 6G9 is shown in FIG. 9. Twenty-four individual colonies were randomly selected from plate, 500 pM BoNT/Gi was used for individual colony screening. Four colonies with the highest binding signal were sent for sequencing, shown in FIG. 10.

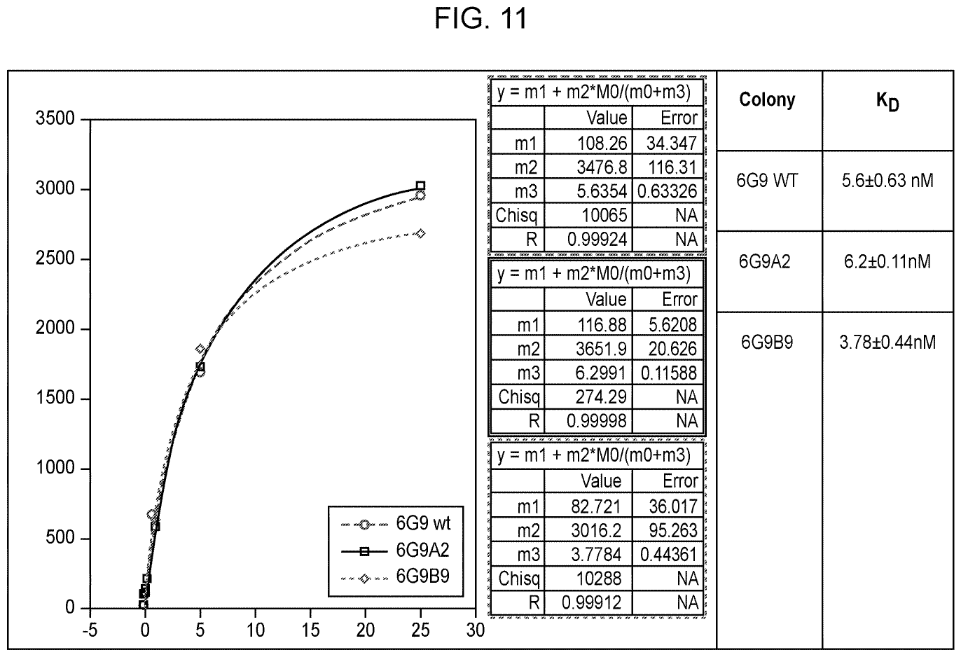

Colonies revealed two different sequences 9A2 and 9B2=9C2-9D10. The Kp of these two was measured on yeast surface using flow cytometry (FIG. 11).

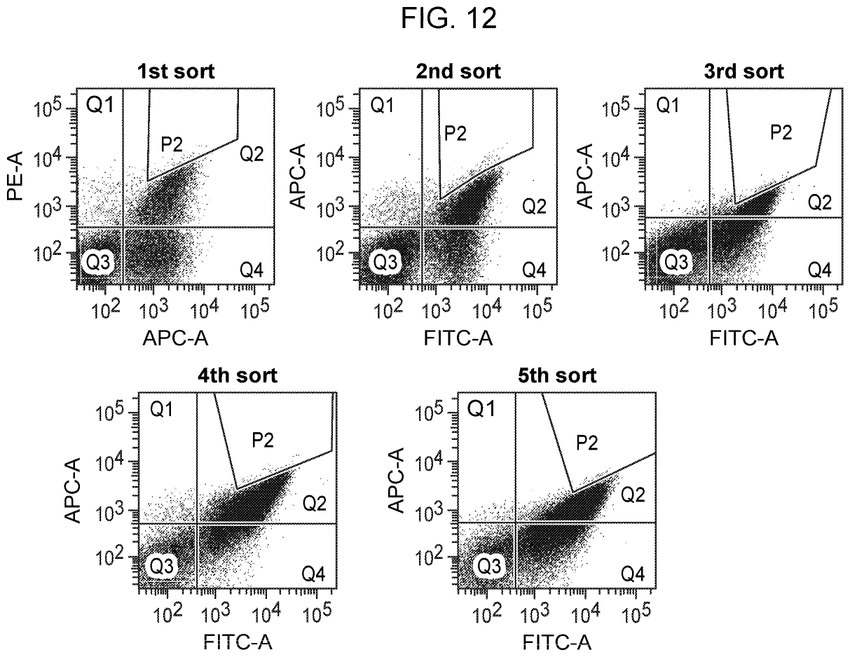

The colony 6G9B8 was named hu6G9 and was then affinity matured using error prone PCR mediated random mutation. The library was displayed on the surface of yeast and contained more than 7×10⁷ variants. Library sorting was done using decreasing BoNT/Gi concentrations as shown in FIG. 12. 1^{st} sort: 50 nM Gi+6G7.1-647; 2^{nd} sort: 10 nM Gi+6G10-GoatAntiHumanPE; 3^{rd} sort: 2 nM Gi+6G10-647; 4^{th} sort: 0.4 nM Gi+6G10-647; 5^{th} sort: 0.2 nM Gi, incubated at room temperature for 1 hour then incubated at room temperature for four hours with 6G9 IgG and detection with 6G10-647.

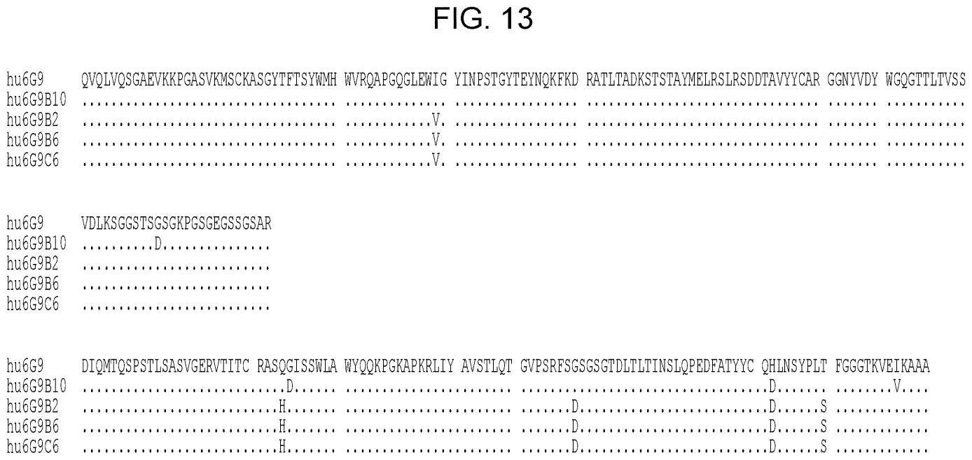

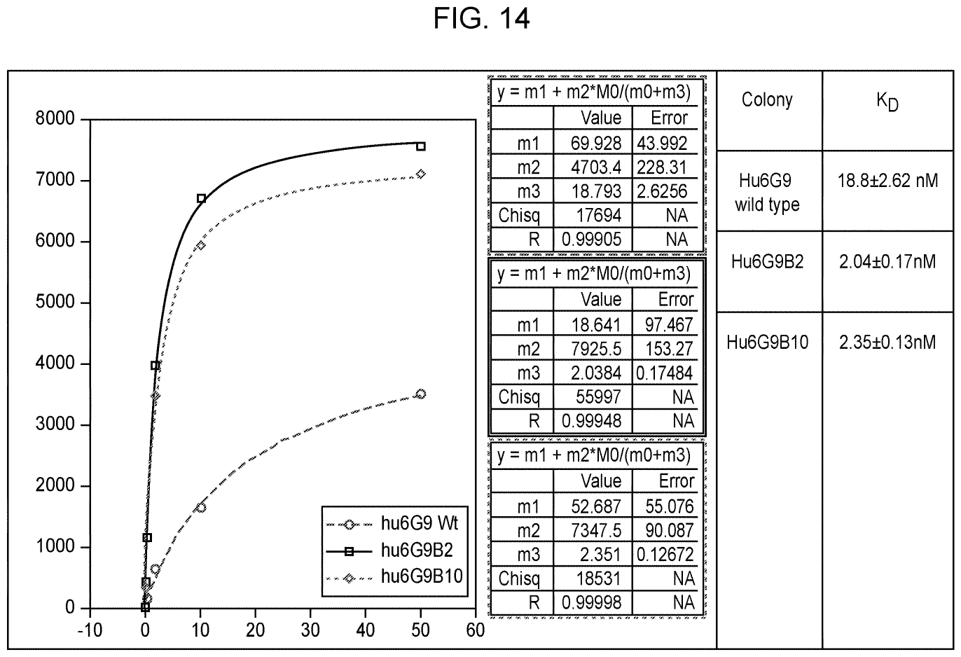

VK sequencing of the colonies from the 5^{th} sort was performed and is shown in FIG. 13. Sequencing identified two different colonies. hu6G9B10 which had mutations VK-CDR1, G→D, VK-CDR3, H→D; VK-FW4, I→V; and hu6G9B2 which had mutations VK-CDR1, Q→H, VK-FW3, G→D; VK-CDR3, H→D, T→S; VH-CDR1, I→V. Affinity of these two mutants was determined, as shown in FIG. 14.

K_{D} values of scFv showed a 9-fold improvement in affinity on the yeast surface for the two variants hu6G9B2 with a K_{D} of 2.04±0.17 nM and 6G9B10 with a Kp of 2.35±0.13 nM compared to hu6G9 WT with K_{D} of 18.8±2.62 nM. hu6G9B2 was selected for further development and conversion to IgG. This was named IgG hu6G9.1.

### Example 5

### 6G10 Humanization and Affinity Maturation

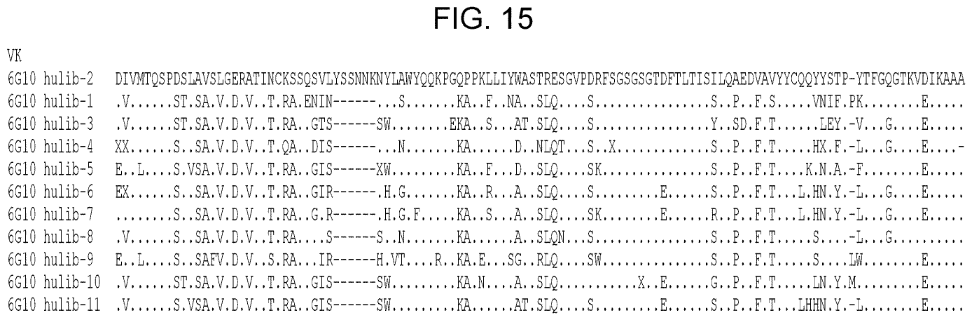

VH fragments of 6G10 (30 microgram of hVH) were prepared and cloned into the pYD4-huVK library (80 microgram of pYD4-huVK). The resulting library size was 1×10⁷. Twelve colonies were picked from each library for sequence analysis to assess diversity. As shown in FIG. 15, the sequence diversity of the library was good.

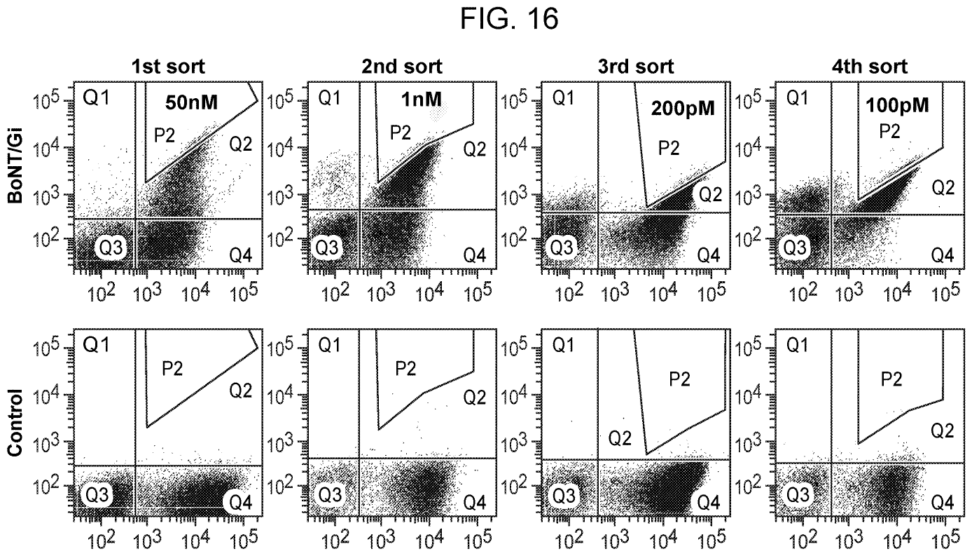

The library was enriched for four rounds with FACS sorting as showed in FIG. 16.

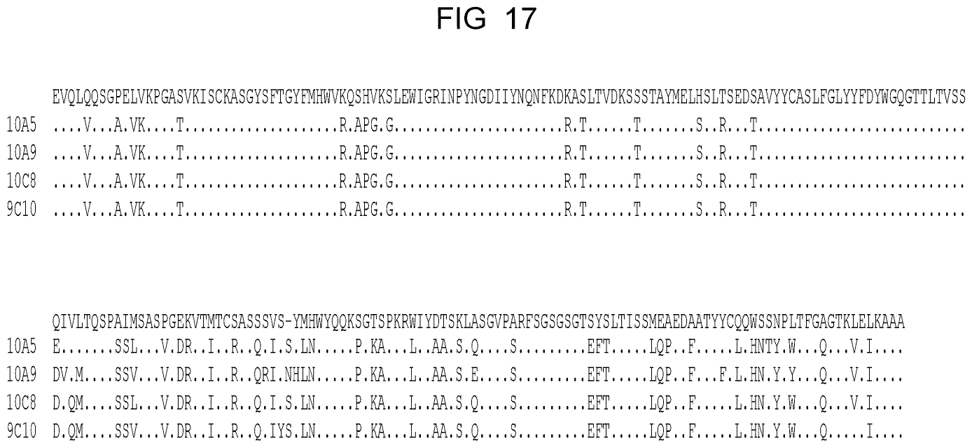

Individual colonies were screened using 500 pM BoNT/Gi. The 4 colonies with the highest binding signal were then sequenced (FIG. 17).

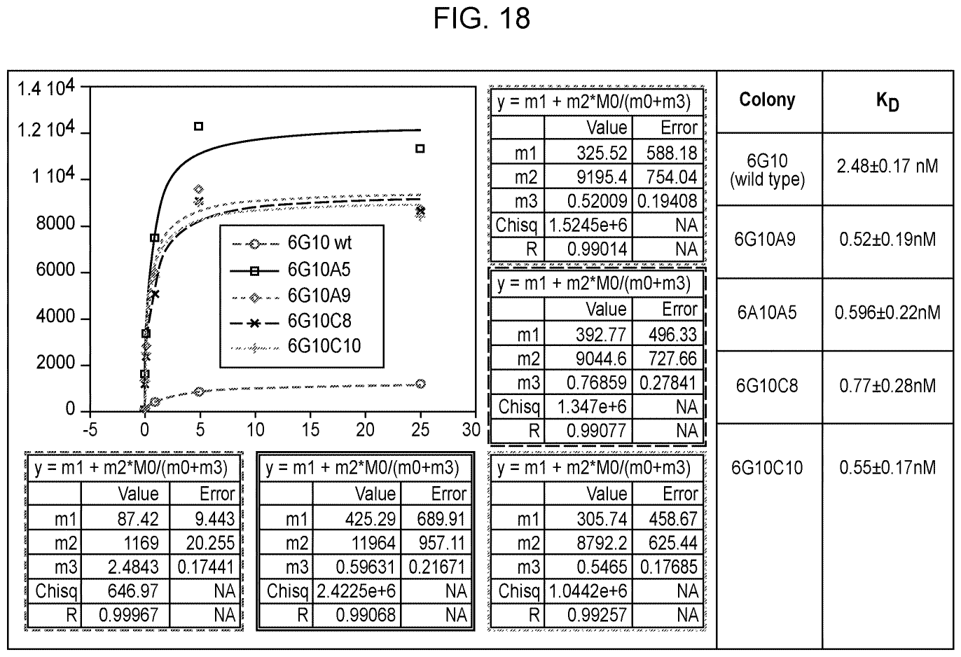

The four colonies had unique sequences and their Kp values were determined on yeast surface with flow cytometry as shown in FIG. 18.

### Example 6

### 6G7 Humanization and Affinity Maturation

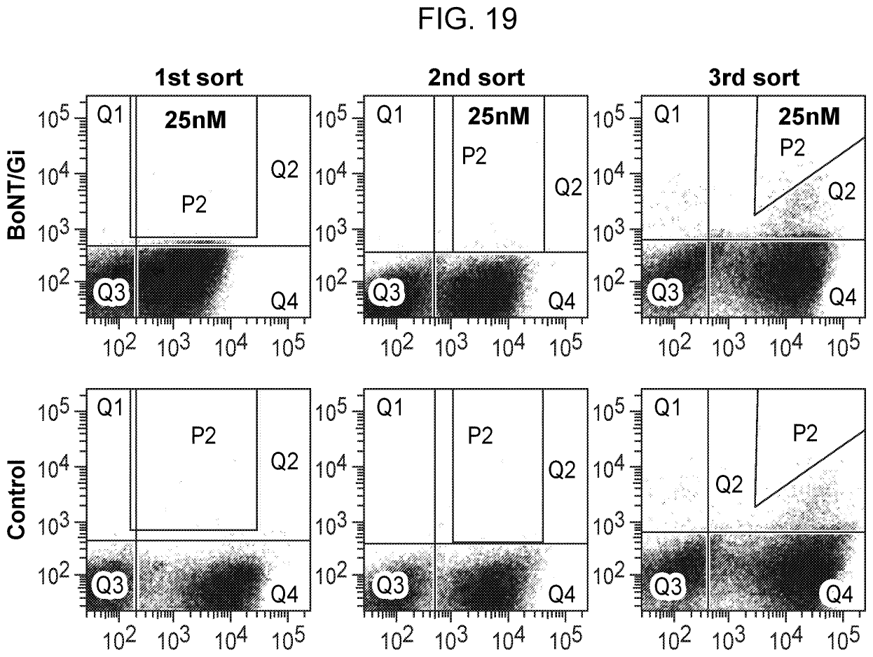

Three different methods were tried to humanize 6G7.1. The first method tried was to humanize and synthesize the VH fragment, then put the huVH into a human VK library to form a human VK shuffling library and use FACS sorting for enrichment (FIG. 19). The results of this approach are shown in FIG. 5. However, no positive binders were identified using this strategy.

A second method was then tried for humanization. hu VK was synthesized and synthesized huVH and huVK cloned into pYD4 to form pYD4-hu6G7.1, which was then displayed on yeast. The Kp on yeast surface was measured side by side with the murine 6G7.1, which has a K_{D} of 2.1 nM for BoNT/Gi. The results was a loss of BoNT/Gi binding upon humanization. The K_{D} of hu6G7.1 was too low and not determined. The signal intensity of binding is shown in Table 9.

Due to lack of binding of the hu6G7.1, the sequence was then examined. A potential glycosylation site was identified in the VK framework 3 region (sequence NDT). The sequence was then mutated to EDT to remove the glycosylation site. The new antibody was named hu6G7.1E.

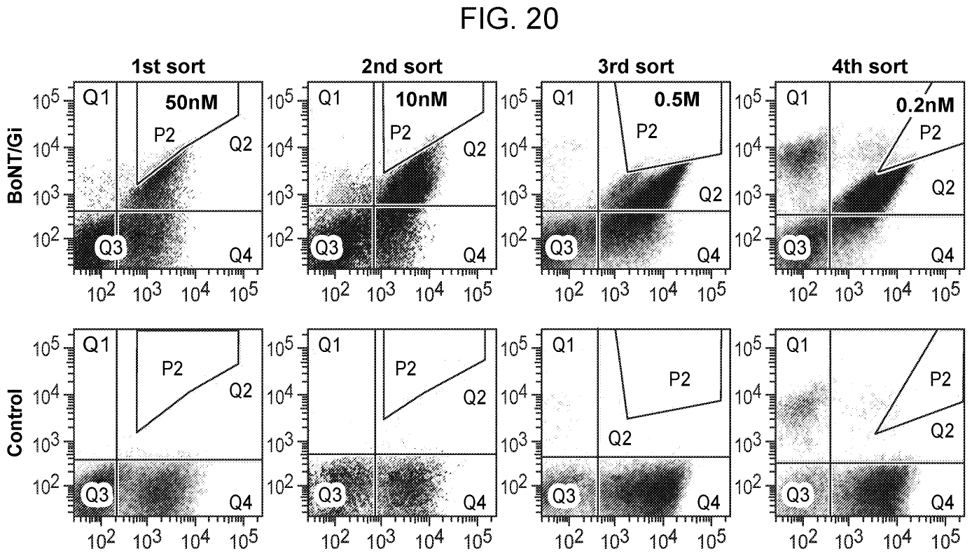

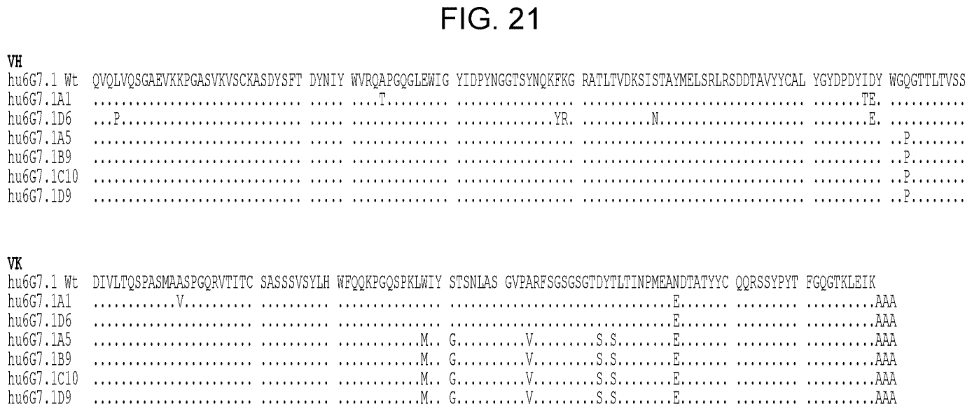

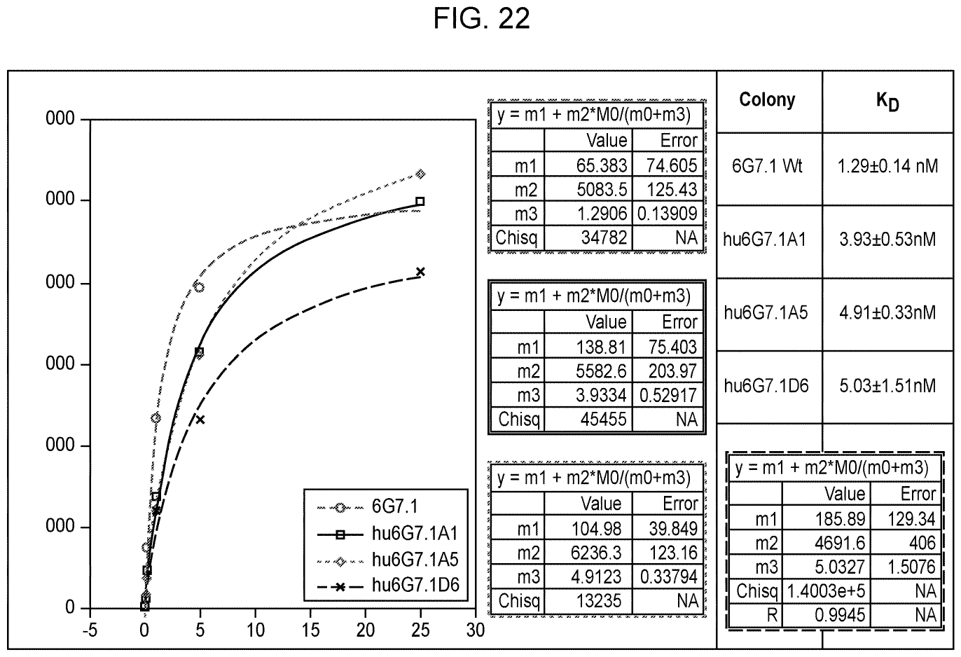

Based on the colony hu6G7.1E, a random mutation library was made to improve the affinity of hu6G7.E (FIG. 22). The library was screened as shown in FIG. 6 and the results are shown in FIG. 20.

As shown in FIG. 8, the colonies A5, B9, C10, D8 are the same. K_{D} values of the colonies on yeast were then measured and it was found that clone A1 is the best binder (FIG. 22).

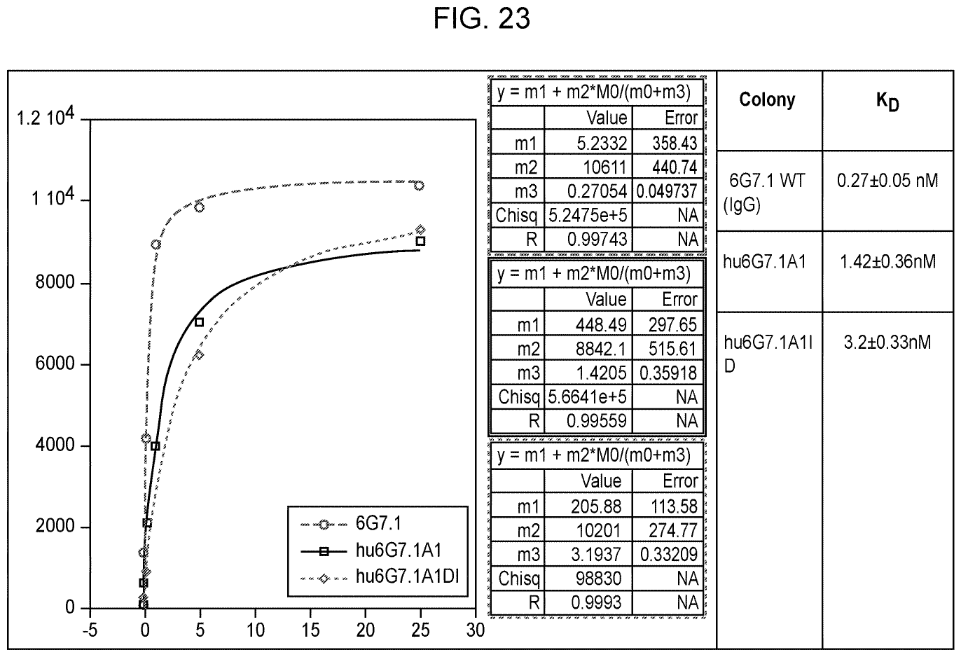

Analysis of the sequence of A1 revealed that the VH CDR3 was mutated from the wild type sequence “YGYDPDYIDY” (SEQ ID NO:40) to YGYDPDYTEY” (SEQ ID NO:47). As this could be the reason for the decreased affinity, TE was mutated back to ID. However, back mutation of TE to ID further lowered the affinity 2-3× and is 10× lower than the murine 6G7.1 (FIG. 23). Therefore, both hu6G7.1A1 and hu6G7.1A1ID were expressed as IgG and the final antibody was selected based on expression yield and affinity.

TABLE 9
Yeast displayed humanized 6G7.1 vs. murine 6G7.1

| BoNT/Gi | | | | | | |
|---|---|---|---|---|---|---|
| concentration (nM) | 0 | 0.04 | 0.2 | 1.0 | 5.0 | 25 |
| 6G7.1 signal | 38 | 116 | 401 | 1094 | 2099 | 2961 |
| intensity | | | | | | |
| hu6G7.1 signal | 43 | 43 | 51 | 296 | 130 | 232 |
| intensity | | | | | | |

### Example 7

### Hu6G11 Affinity Measurements

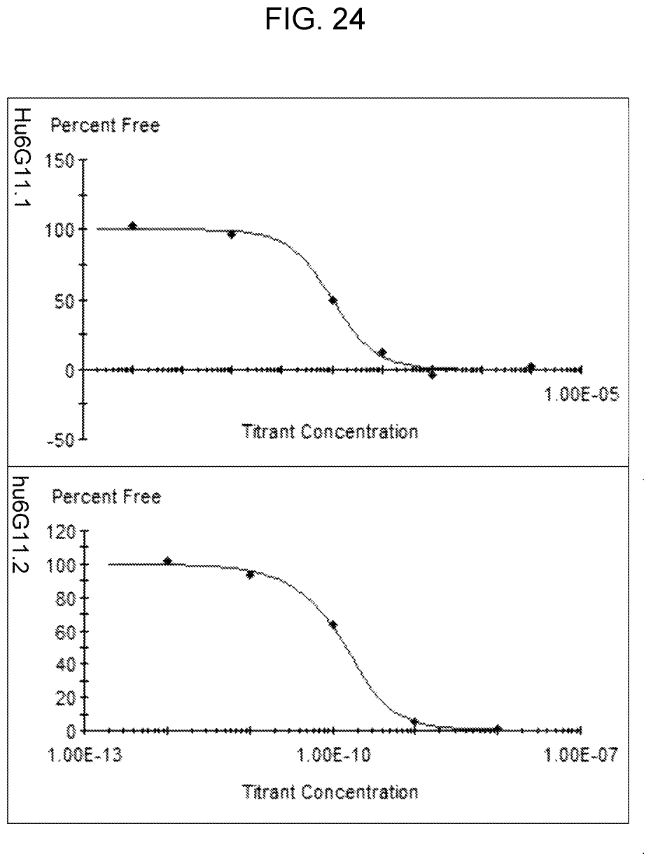

The IgG format of the affinity matured G mAbs was produced. hu6G11.1 and hu6G11.2 affinity matured from hu6G11 had K_{D} values of 100.7 (120.15-68.42 PM) and 48 pM (79.66 pM-22 pM) respectively. FIG. 24 shows results from the range finding KinExA experiments.

With the above strategies and as summarized in FIG. 25, 6G6, 6G7, 6G9, 6G10 and 6G11 were all humanized and affinity maturated to become Hu6G6.2, Hu6G7.2, Hu6G9.1, Hu6G10 and Hu6G11.2. Some were humanized first by selection of light chain shuffling libraries consisting of humanized VH with a fully human VK repertoire, then using random mutagenesis for further improvement. Others were humanized by selection of heavy chain shuffling with a fully humanized V_{L} and site-directed mutagenesis, then using random mutagenesis for further improvement.

6G6 humanization library was enriched for four rounds of FACS sorting, with decreased crude BoNT/G toxin concentration from 1:50 in the first round to 1:300 in the fourth round of sorting. The humanized mAb hu6G6 showed improved affinity on yeast surface of 5.0 nM, increasing at least three-fold compared with the parental 6G6, 17.7 nM. hu6G6 was transformed to IgG, with the K_{D} measured with KinExA 8.37 nM. 6G9 humanization library was also enriched for four rounds of FACS sorting. Fifty nM of BoNT/Gi was applied for the first and seconds of FACS sorting, 10 nM for the third round, and 2.5 nM for the fourth round. The Kp value of humanized 6G9 (hu6G9) was measured on yeast surface with flow cytometry as 3.78 nM, compared with 6G9, 5.6 nM.

Random mutagenesis libraries were made to improve the affinity of hu6G6 and hu6G9. For hu6G6 mutagenesis library, 25 nM BoNT/Gi was applied for the first round of isolation, and 1 nM BoNT/Gi for the second round of sorting. In the third round of enrichment, a k_{off} screening was performed by 5 nM BoNT/Gi saturation followed with wild type hu6G6 competition for four hours. The collected population was enriched for one more round with 200 pM BoNT/Gi, resulting the improved colony hu6G6.1, with K_{D} on yeast surface 1.15±0.14 nM and the IgG K_{D} value measured with KinExA 476 pM, with 18-fold improvement compared with the hu6G6. A new library made by random mutagenesis based on Hu6G6.1 and 4 rounds of BoNT/Gi sorting resulted to the identification of the mAb Hu6G6.2 with a yeast scFv KD of 219.78 pM and IgG KD of 42.2 pM. For hu6G9 mutagenesis library, five rounds of enrichment with decreased BoNT/Gi concentration of 50 nM Gi, 10 nM Gi, 2 nM BoNT/Gi, 400 pM and 200 pM. The K_{D} of the resulting hu6G9.1 was measured as 2.04±0.17 nM on yeast surface, compared to the wild type hu6G9, 18.8±2.62 nM, with 9-fold improvement. The IgG K_{D} of hu6G9.1 in solution was measured as 27.48 pM.

6G10 humanization library was enriched with 50 nM BoNT/Gi for the first round of sorting, then decreased BoNT/Gi concentration 1 nM, 200 pM and 100 pM was applied in the second, third and fourth rounds of sorting. Four different colonies were identified, with the Kp value on yeast surface 0.59 nM, 0.52 nM, 0.77 nM and 0.55 nM, with five-fold improvement compared with the mouse scFv 6G10, 2.48 nM. IgG was prepared for one of the colonies as hu6G10, with KinExA measured K_{D} 8.36 pM, with thirty-nine-fold improvement compared with 6G10, 330 pM. After humanization, hu6G10 had high affinity to BoNT/G (IgG K_{D}, 8.36 pM).

6G11 humanization library was screened for five rounds of FACS sorting, with BoNT/Gi concentration being decreased from 50 nM at the first round to 0.5 nM at the last round of sorting. The humanized mAb huG11 showed 25-fold improvement in BoNT/Gi binding (4.57 nM vs. 102.5 nM). The IgG K_{D} in solution was measured as 531 pM. Hu6G11 was further affinity matured via random mutagenesis to Hu6G11.1 (100.7 pM as IgG) and Hu6G11.2 (48 PM as IgG). With the strategy of light chain shuffling, the humanized mAbs contained only 8-10 mouse residues in VH and the affinity were significantly improved.

However, humanization of 6G7 failed with the same strategy in that nothing was obtained from the library, even when sorted with 20 nM of BoNT/Gi. This could be resulted from the absence of compatible light chain to match the humanized VH in the human VK repertoire used in the experiments.

Since humanization of 6G7 failed with light chain shuffling, the mouse mAb was first improved by screening a mutagenesis library. Four rounds of FACS sorting with decreased concentration of BoNT/Gi from 50 nM for the first round to 0.5 nM for the fourth round pointed to the colony 6G7.1, with the scFv K_{D} value measured on yeast surface, 0.69±0.19 nM, and IgG K_{D} measured with KinExA, 26.6 pM. 6G7.1 was then humanized by replacing the non-critical mouse amino acids and keeping the critical mouse one in both VH and VK to hu6G7. A low-rate mutation was introduced into hu6G7 by error prone PCR to make a random mutagenesis library. The library was enriched with four rounds of sorting with decreasing concentrations of BoNT/Gi (50 nM, 10 nM, 0.5 nM and 0.2 nM). From the library, hu6G7.1 and hu6G7.2 were identified with the yeast K_{D} 1.42 nM and 3.2 nM. The K_{D} values of IgGs measured with KinExA was 1.19 nM and 51.01 pM respectively. In both hu6G7.1 and hu6G7.2, a total of 15 mouse residue remained, 7 in VH and 8 in VK.

Taken together, five mAbs against BoNT/G with non-overlapping epitope hu6G6.2, hu6G7.2, hu6G9.1, hu6G10 and hu6G11.2 were identified. Their affinity to the active BoNT/G holotoxin with IgG K_{D} values ranged from 51 to 8.36 pM.

### Example 8

### Determination of the LD 50 of Recombinant BoNT/G

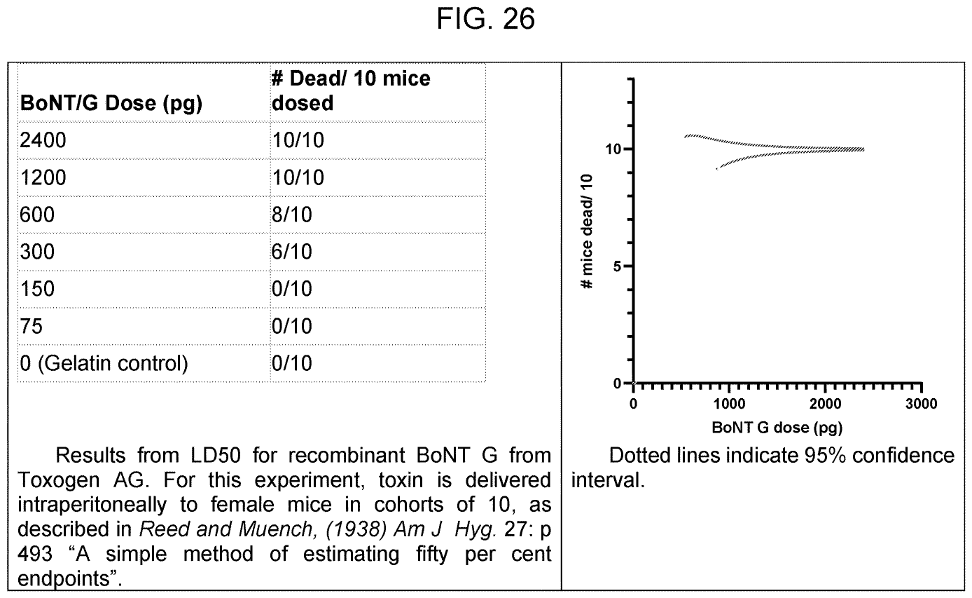

The LD₅₀ of recombinant BoNT G was determined with 10 mice per cohort. The mouse LD₅₀ was determined to be 289 pg (95% CI 234-374 pg) using a curve fit of the data. Thus 10,000 M LD₅₀=2.89 μg/mouse. Results are shown in Table 10 and FIG. 26.

### Example 9

### In Vivo Mouse Neutralization Assay

Previous studies indicated that three mAbs with high affinity and non-overlapping epitopes significantly improved the potency of in vivo BoNT neutralization. All three-mAb combinations of Hu6G6.2, Hu6G7.2, hu6G9.1, hu6G10 and hu6G11.2 were tested in mice exposed with 10,000 MLD₅₀s of recombinant BoNT/G toxin. As shown in Table 10, any three mAb combination of the five antibodies protected mice in the MNA at a dose of 12.5 mg/mouse.

TABLE 10
Mouse neutralization assay (MNA) comparing antibody
combinations against 10,000 LD₅₀ of BoNT/G

| | Number of deaths/number | Number of deaths/number |
|---|---|---|
| | mice treated with | mice treated with |
| Antibodies | 12.5 μg of antibodies | 6.25 μg of antibodies |
| Control | 5/5 | 5/5 |
| (Toxogen BoNT/G only) | | |
| hu6G7.2, hu6G9.1, hu6G10 | 1/5 | 5/5 |
| hu6G6.2, hu6G7.2, hu6G9.1 | 0/5 | 0/5 |
| hu6G6.2, hu6G7.2, hu6G10 | 0/5 | 0/5 |
| hu6G6.2, hu6G7.2, hu6G11.2 | 0/5 | 4/5 |
| hu6G67.2, hu6G9.1, hu6G11.2 | 3/5 | 5/5 |
| hu6G9.1, hu6G10, hu6G11.2 | 1/5 | 5/5 |
| hu6G6.2, hu6G9.1, hu6G10 | 1/5 | 0/5 |
| hu6G6.2, hu6G9.1, hu6G11.2 | 0/5 | 0/5 |
| hu6G6.2, hu6G10, hu6G11.2 | 0/5 | 0/5 |
| hu6G7.2, hu6G10, hu6G11.2 | 0/5 | 3/5 |

What is claimed is:

1. An isolated antibody that specifically binds to a Botulinum neurotoxin serotype BoNT/G, wherein the antibody competes for binding to BoNT/G with an antibody comprising:
a) a variable heavy (V_{H}) chain comprising heavy complementarity determining regions 1-3 (HCDRs 1-3) and a variable light (V_{L}) chain comprising light CDRs 1-3 (LCDRs 1-3) of a pair of V_{H} chain and V_{L} chain of an antibody listed in Table 1, Table 2, Table 3, Table 4, Table 5, or Table 6; or
b) a V_{H} chain comprising the amino acid sequence of the V_{H} chain and a V_{L} chain comprising the amino acid sequence of a pair of V_{H} chain and V_{L} chain of an antibody listed in Table 1, Table 2, Table 3, Table 4, Table 5, or Table 6.

2. The isolated antibody of claim 1, wherein the isolated antibody competes for binding to BoNT/G with an antibody comprising:
a V_{H} chain comprising HCDRs 1-3 and a V_{L} chain comprising LCDRs 1-3 of a pair of V_{H} chain and V_{L} chain of an antibody listed in Table 1, Table 2, Table 3, Table 4, Table 5, or Table 6.

3. The isolated antibody of claim 2, wherein the isolated antibody competes for binding to BoNT/G with an antibody comprising:
a V_{H} chain comprising HCDRs 1-3 and a V_{L} chain comprising LCDRs 1-3 of a pair of V_{H} chain and V_{L} chain of the Hu6G6.2 antibody, the Hu6G7.2 antibody, the Hu6G9.1 antibody, the Hu6G10 antibody, or the Hu6G11.2 antibody.

4. The isolated antibody of claim 3, wherein the isolated antibody competes for binding to BoNT/G with an antibody comprising:
a V_{H} chain comprising HCDRs 1-3 and heavy chain framework regions 1-4 (H-FRs 1-4) and a V_{L} chain comprising LCDRs1-3 and light chain framework regions 1-4 (L-FRs 1-4) of the Hu6G6.2 antibody, the Hu6G7.2 antibody, the Hu6G9.1 antibody, the Hu6G10 antibody, or the Hu6G11.2 antibody.

5. The isolated antibody of claim 1, wherein the isolated antibody competes for binding to BoNT/G with an antibody comprising:
a V_{H} chain comprising the amino acid sequence of the V_{H} chain and a V_{L} chain comprising the amino acid sequence of a pair of V_{H} chain and V_{L} chain of an antibody listed in Table 1, Table 2, Table 3, Table 4, Table 5, or Table 6.

6. The isolated antibody of claim 5, wherein the isolated antibody competes for binding to BoNT/G with an antibody comprising:
a V_{H} chain comprising the amino acid sequence of the V_{H} chain and a V_{L} chain comprising the amino acid sequence of the V_{L} chain of a pair of V_{H} chain and VI chain of the Hu6G6.2 antibody, the Hu6G7.2 antibody, the Hu6G9.1 antibody, the Hu6G10 antibody, or the Hu6G11.2 antibody.

7. The isolated antibody of claim 6, wherein the isolated antibody competes for binding to BoNT/G with an antibody comprising:
at least 80%, at least 90%, or at least 95% sequence identity to a V_{H} chain comprising the amino acid sequence of the V_{H} chain and a V_{L} chain comprising the amino acid sequence of the V_{L} chain of a pair of V_{H} chain and V_{L} chain of the Hu6G6.2 antibody, the Hu6G7.2 antibody, the Hu6G9.1 antibody, the Hu6G10 antibody, or the Hu6G11.2 antibody.

8. An isolated antibody that binds to a Botulinum neurotoxin serotype BoNT/G, wherein the antibody comprises:
a) a variable heavy (V_{H}) chain comprising heavy complementarity determining regions 1-3 (HCDRs 1-3) and a variable light (V_{L}) chain comprising light CDRs 1-3 (LCDRs 1-3) of a pair of V_{H} chain and V_{L} chain of an antibody listed in Table 1, Table 2, Table 3, Table 4, Table 5, or Table 6;
b) a V_{H} chain comprising the amino acid sequence of the V_{H} chain and a V_{L} chain comprising the amino acid sequence of the V_{L} chain of a pair of V_{H} chain and V_{L} chain of an antibody listed in Table 1, Table 2, Table 3, Table 4, Table 5, or Table 6;
c) a V_{H} chain comprising the amino acid sequence of the V_{H} chain of an antibody listed in Table 1, Table 2, Table 3, Table 4, Table 5, or Table 6;
d) a V_{L} chain comprising the amino acid sequence of the V_{L} chain of an antibody listed in Table 1, Table 2, Table 3, Table 4, Table 5, or Table 6;
e) a V_{H} chain comprising HCDRs 1-3 of an antibody listed in Table 1, Table 2, Table 3, Table 4, Table 5, or Table 6; or
f) a V_{L} chain comprising LCDRs 1-3 of an antibody listed in Table 1, Table 2, Table 3, Table 4, Table 5, or Table 6.

9. The isolated antibody of claim 8, wherein the antibody comprises:
a V_{H} chain comprising HCDRs 1-3 and a V_{L} chain comprising LCDRs 1-3 of a pair of V_{H} chain and V_{L} chain of an antibody listed in Table 1, Table 2, Table 3, Table 4, Table 5, or Table 6.

10. The isolated antibody of claim 9, wherein the antibody comprises:
a V_{H} chain comprising the amino acid sequence of the V_{H} chain and a V_{L} chain comprising the amino acid sequence of the V_{L} chain of a pair of V_{H} chain and V_{L} chain of an antibody listed in Table 1, Table 2, Table 3, Table 4, Table 5, or Table 6.

11. The isolated antibody of claim 9, wherein the antibody comprises:
a V_{H} chain comprising an amino acid sequence having at least 80%, at least 90%, or at least 95% sequence identity to a V_{H} chain and a V_{L} chain comprising an amino acid sequence having at least 80%, at least 90%, or at least 95% sequence identity to a V_{L} chain of the Hu6G6.2 antibody, the Hu6G7.2 antibody, the Hu6G9.1 antibody, the Hu6G10 antibody, or the Hu6G11.2 antibody.

12. The isolated antibody of claim 9, wherein the antibody comprises:
a V_{H} chain and a V_{L} chain of the Hu6G6.2 antibody, the Hu6G7.2 antibody, the Hu6G9.1 antibody, the Hu6G10 antibody, or the Hu6G11.2 antibody.

13. The isolated antibody of any one of claim 1, 2, 5, or 8-10, wherein the antibody is a human antibody or a humanized antibody.

14. The isolated antibody of any one of claims 1-13, wherein the antibody is a single chain Fv (scFv), IgG, Fab, (Fab′)₂, or (scFv′)₂.

15. The isolated antibody of any one of claims 1-14, wherein the antibody binds to BoNT/G with a K_{D} of about 5 nM or less.

16. A composition comprising:
a pharmaceutically acceptable carrier; and
at least a first antibody according to any one of claims 1-15.

17. The composition of claim 16, comprising a first antibody and a second antibody according to any one of claims 1-15.

18. The composition of claim 17, wherein the first antibody binds to a first epitope of BoNT/G and the second antibody binds to a second epitope of BoNT/G.

19. An isolated nucleic acid comprising a nucleotide sequence encoding the amino acid sequence of any one of the antibodies of any one of claims 1-15.

20. A vector comprising the isolated nucleic acid of claim 19.

21. A first nucleic acid comprising a nucleotide sequence encoding the amino acid sequence of the V_{H} chain and a second nucleic acid comprising a nucleotide sequence encoding the amino acid sequence of the V_{L} chain any one of the antibodies of any one of claims 1-15.

22. A first vector comprising the first nucleic acid and a second vector comprising the second nucleic acid of claim 21.

23. A host cell comprising the nucleic acid of claim 19, the vector of claim 20, the first and second nucleic acids of claim 21 or the first and second vectors of claim 22.

24. The isolated antibody according to any one of claims 1-15, wherein the antibody is labeled.

25. A method of specifically binding to a Botulinum neurotoxin in a mammal comprising:
administering to the mammal an effective amount of an antibody of any one of claims 1-15;
wherein the administering provides for specific binding of Botulinum neurotoxin present in the mammal.

26. A method of specifically binding to a Botulinum neurotoxin in a mammal, comprising:
administering to the mammal an effective amount of a composition of any one of claims 16-18;
wherein the administering provides for specific binding of Botulinum neurotoxin present in the mammal.

27. A method for detecting Botulinum neurotoxin in a sample, comprising:
contacting an antibody of any one of claims 1-15 with the sample,
detecting binding of said antibody to Botulinum neurotoxin in the sample.

28. The method of claim 27, wherein the antibody is labeled.

29. The method of claim 28, wherein said label is a fluorescent label, a chemiluminescent label, a radiolabel, an enzyme, or a chromogenic label.

30. The method of any one of claims 27-29, wherein said sample is blood.

31. The method of any one of claims 27-30, wherein the antibody is immobilized on a substrate.

32. The method of any one of claims 27-31, wherein the antibody is conjugated to a nucleic acid and wherein said detecting comprises a polymerase chain reaction.

33. A method of reducing the likelihood that an individual at risk of exposure to Botulinum neurotoxin will experience symptoms of Botulinum neurotoxin poisoning following exposure, the method comprising administering to said individual an effective amount of an antibody of any one of claims 1-15;
wherein said administering provides for reducing the likelihood that the individual will experience symptoms of Botulinum neurotoxin poisoning.


SEQUENCE LISTING (278 sequences)

SEQ ID NO: 1 - protein, 121 aa, synthetic construct
EVQLQQSGAELVKPGASVKLSCTASGFNIKDTYMHWVKQRPEQGLEWIGRIDPANGNTKYDPKFQGKATITADTSSNTAYLQLSSLTSEDTAVYYCASGPNYYGTTWFAYWGQGTTVTVSS

SEQ ID NO: 2 - protein, 30 aa, synthetic construct
EVQLQQSGAELVKPGASVKLSCTASGFNIK

SEQ ID NO: 3 - protein, 5 aa, synthetic construct
DTYMH

SEQ ID NO: 4 - protein, 14 aa, synthetic construct
WVKQRPEQGLEWIG

SEQ ID NO: 5 - protein, 17 aa, synthetic construct
RIDPANGNTKYDPKFQG

SEQ ID NO: 6 - protein, 32 aa, synthetic construct
KATITADTSSNTAYLQLSSLTSEDTAVYYCAS

SEQ ID NO: 7 - protein, 12 aa, synthetic construct
GPNYYGTTWFAY

SEQ ID NO: 8 - protein, 11 aa, synthetic construct
WGQGTTVTVSS

SEQ ID NO: 9 - protein, 121 aa, synthetic construct
QVQLVQSGAEVKKPGASVKLSCKASGFNIKDTYMHWVRQAPGQGLEWIGRIDPANGNTKYDPKFQGRATITADTSINTAYLELSRLRSDDTAVYYCASGPNYYGTTWFAYWGQGTTVTVSS

SEQ ID NO: 10 - protein, 30 aa, synthetic construct
QVQLVQSGAEVKKPGASVKLSCKASGFNIK

SEQ ID NO: 11 - protein, 14 aa, synthetic construct
WVRQAPGQGLEWIG

SEQ ID NO: 12 - protein, 32 aa, synthetic construct
RATITADTSINTAYLELSRLRSDDTAVYYCAS

SEQ ID NO: 13 - protein, 121 aa, synthetic construct
QVQLVQSGAEVKKPGASVKLSCKASGFNIKDTYMHWVRQAPGQGLEWIGRIDPENGNTKYDPKFQGRATITAYKSINTAYLELSRLRSDDTAVYYCASGPNYYGTTWFAYWGQGTTVTVSS

SEQ ID NO: 14 - protein, 17 aa, synthetic construct
RIDPENGNTKYDPKFQG

SEQ ID NO: 15 - protein, 32 aa, synthetic construct
RATITAYKSINTAYLELSRLRSDDTAVYYCAS

SEQ ID NO: 16 - protein, 121 aa, synthetic construct
QVQLVQSGAEVKKPGASVKLSCKASGFNIKDTYMHWVRQAPGQGLEWIGRIDPENGNTKYDPKFQGRATITAYKSINRAYLELSRLRSDDTAVYYCASGPNYYGTTWFAYWGQGTTVTVSS

SEQ ID NO: 17 - protein, 32 aa, synthetic construct
RATITAYKSINRAYLELSRLRSDDTAVYYCAS

SEQ ID NO: 18 - protein, 107 aa, synthetic construct
QIVLTQSPAILSASPGEKVTMTCRASSSVSYMHWYQQKPGSSPRLWIYRTSNLASGVPARFSGSGSGTSYSLTISSMEAEDAATYYCQQYHSYPRTFGGGTKLEIKR

SEQ ID NO: 19 - protein, 23 aa, synthetic construct
QIVLTQSPAILSASPGEKVTMTC

SEQ ID NO: 20 - protein, 10 aa, synthetic construct
RASSSVSYMH

SEQ ID NO: 21 - protein, 15 aa, synthetic construct
WYQQKPGSSPRLWIY

SEQ ID NO: 22 - protein, 7 aa, synthetic construct
RTSNLAS

SEQ ID NO: 23 - protein, 32 aa, synthetic construct
GVPARFSGSGSGTSYSLTISSMEAEDAATYYC

SEQ ID NO: 24 - protein, 9 aa, synthetic construct
QQYHSYPRT

SEQ ID NO: 25 - protein, 11 aa, synthetic construct
FGGGTKLEIKR

SEQ ID NO: 26 - protein, 109 aa, synthetic construct
EIVLTQSPSTLSASVGDRVTITCRASHGISNYLAWYQQKPGRAPKLLIYKASTLHIGVPSRFSGSGSGTEFALTISSLQPDDFSTYYCQQYNSYLPHTFGQGTRLEIKR

SEQ ID NO: 27 - protein, 23 aa, synthetic construct
EIVLTQSPSTLSASVGDRVTITC

SEQ ID NO: 28 - protein, 11 aa, synthetic construct
RASHGISNYLA

SEQ ID NO: 29 - protein, 15 aa, synthetic construct
WYQQKPGRAPKLLIY

SEQ ID NO: 30 - protein, 7 aa, synthetic construct
KASTLHI

SEQ ID NO: 31 - protein, 32 aa, synthetic construct
GVPSRFSGSGSGTEFALTISSLQPDDFSTYYC

SEQ ID NO: 32 - protein, 10 aa, synthetic construct
QQYNSYLPHT

SEQ ID NO: 33 - protein, 11 aa, synthetic construct
FGQGTRLEIKR

SEQ ID NO: 34 - protein, 119 aa, synthetic construct
DVQLQESGPELVKPGASVKVSCKASDYSFTDYNIYWVKQSHGKSLEWIGYIDPYNGGTSYNQKFKGKATLTVDKSSSTAFMYLNSLTSEDSAVYYCALYGYDPDYIDYWGQGTTLTVSS

SEQ ID NO: 35 - protein, 30 aa, synthetic construct
DVQLQESGPELVKPGASVKVSCKASDYSFT

SEQ ID NO: 36 - protein, 5 aa, synthetic construct
DYNIY

SEQ ID NO: 37 - protein, 14 aa, synthetic construct
WVKQSHGKSLEWIG

SEQ ID NO: 38 - protein, 17 aa, synthetic construct
YIDPYNGGTSYNQKFKG

SEQ ID NO: 39 - protein, 32 aa, synthetic construct
KATLTVDKSSSTAFMYLNSLTSEDSAVYYCAL

SEQ ID NO: 40 - protein, 10 aa, synthetic construct
YGYDPDYIDY

SEQ ID NO: 41 - protein, 11 aa, synthetic construct
WGQGTTLTVSS

SEQ ID NO: 42 - protein, 119 aa, synthetic construct
QVQLVQSGAEVKKPGASVKVSCKASDYSFTDYNIYWVRQTPGQGLEWIGYIDPYNGGTSYNQKFKGRATLTVDKSISTAYMELSRLRSDDTAVYYCALYGYDPDYIDYWGQGTTLTVSS

SEQ ID NO: 43 - protein, 30 aa, synthetic construct
QVQLVQSGAEVKKPGASVKVSCKASDYSFT

SEQ ID NO: 44 - protein, 14 aa, synthetic construct
WVRQTPGQGLEWIG

SEQ ID NO: 45 - protein, 32 aa, synthetic construct
RATLTVDKSISTAYMELSRLRSDDTAVYYCAL

SEQ ID NO: 46 - protein, 119 aa, synthetic construct
QVQLVQSGAEVKKPGASVKVSCKASDYSFTDYNIYWVRQTPGQGLEWIGYIDPYNGGTSYNQKFKGRATLTVDKSISTAYMELSRLRSDDTAVYYCALYGYDPDYTEYWGQGTTLTVSS

SEQ ID NO: 47 - protein, 10 aa, synthetic construct
YGYDPDYTEY

SEQ ID NO: 48 - protein, 107 aa, synthetic construct
QIVLTQSPAIMSASPGEKVTITCSASSSVSYLHWFQQKPGTSPKLWIYSTSNLASGVPARFSGSGSGTSYSLTISRMEAEDAATYYCQQRSSYPYTFGGGTKLEIKA

SEQ ID NO: 49 - protein, 23 aa, synthetic construct
QIVLTQSPAIMSASPGEKVTITC

SEQ ID NO: 50 - protein, 10 aa, synthetic construct
SASSSVSYLH

SEQ ID NO: 51 - protein, 15 aa, synthetic construct
WFQQKPGTSPKLWIY

SEQ ID NO: 52 - protein, 7 aa, synthetic construct
STSNLAS

SEQ ID NO: 53 - protein, 32 aa, synthetic construct
GVPARFSGSGSGTSYSLTISRMEAEDAATYYC

SEQ ID NO: 54 - protein, 9 aa, synthetic construct
QQRSSYPYT

SEQ ID NO: 55 - protein, 11 aa, synthetic construct
FGGGTKLEIKA

SEQ ID NO: 56 - protein, 107 aa, synthetic construct
DIVLTQSPASMAVSPGQRVTITCSASSSVSYLHWFQQKPGQSPKLWIYSTSNLASGVPARFSGSGSGTDYTLTINPMEAEDTATYYCQQRSSYPYTFGQGTKLEIKR

SEQ ID NO: 57 - protein, 23 aa, synthetic construct
DIVLTQSPASMAVSPGQRVTITC

SEQ ID NO: 58 - protein, 15 aa, synthetic construct
WFQQKPGQSPKLWIY

SEQ ID NO: 59 - protein, 32 aa, synthetic construct
GVPARFSGSGSGTDYTLTINPMEAEDTATYYC

SEQ ID NO: 60 - protein, 11 aa, synthetic construct
FGQGTKLEIKR

SEQ ID NO: 61 - protein, 116 aa, synthetic construct
EVQLQQSGDDLVKPGASVKMSCKASGYTFTSYWMHWVKQRPGQGLEWIGYINPSTGYTEYNQKFKDKATLTADKSSSTAYMQLSSLTSEDSAVYYCARGGNYVDYWGQGTTLTVSS

SEQ ID NO: 62 - protein, 30 aa, synthetic construct
EVQLQQSGDDLVKPGASVKMSCKASGYTFT

SEQ ID NO: 63 - protein, 5 aa, synthetic construct
SYWMH

SEQ ID NO: 64 - protein, 14 aa, synthetic construct
WVKQRPGQGLEWIG

SEQ ID NO: 65 - protein, 17 aa, synthetic construct
YINPSTGYTEYNQKFKD

SEQ ID NO: 66 - protein, 32 aa, synthetic construct
KATLTADKSSSTAYMQLSSLTSEDSAVYYCAR

SEQ ID NO: 67 - protein, 7 aa, synthetic construct
GGNYVDY

SEQ ID NO: 68 - protein, 116 aa, synthetic construct
QVQLVQSGAEVKKPGASVKMSCKASGYTFTSYWMHWVRQAPGQGLEWVGYINPSTGYTEYNQKFKDRATLTADKSTSTAYMELRSLRSDDTAVYYCARGGNYVDYWGQGTTLTVSS

SEQ ID NO: 69 - protein, 30 aa, synthetic construct
QVQLVQSGAEVKKPGASVKMSCKASGYTFT

SEQ ID NO: 70 - protein, 14 aa, synthetic construct
WVRQAPGQGLEWVG

SEQ ID NO: 71 - protein, 32 aa, synthetic construct
RATLTADKSTSTAYMELRSLRSDDTAVYYCAR

SEQ ID NO: 72 - protein, 107 aa, synthetic construct
EIVLTQSPAIMSASPGEKVTISCSASSSVSYMYWYQQKPGSSPKPWIYRTSNLASGVPARFSGSGSGTSYSLTISSMEAEDAATYYCQQWSSNPYTFGGGTKLEIKR

SEQ ID NO: 73 - protein, 23 aa, synthetic construct
EIVLTQSPAIMSASPGEKVTISC

SEQ ID NO: 74 - protein, 10 aa, synthetic construct
SASSSVSYMY

SEQ ID NO: 75 - protein, 15 aa, synthetic construct
WYQQKPGSSPKPWIY

SEQ ID NO: 76 - protein, 9 aa, synthetic construct
QQWSSNPYT

SEQ ID NO: 77 - protein, 108 aa, synthetic construct
DIQMTQSPSTLSASVGERVTITCRASHGISSWLAWYQQKPGKAPKRLIYAVSTLQTGVPSRFSDSGSGTDLTLTINSLQPEDFATYYCQDLNSYPLSFGGGTKVEIKR

SEQ ID NO: 78 - protein, 23 aa, synthetic construct
DIQMTQSPSTLSASVGERVTITC

SEQ ID NO: 79 - protein, 11 aa, synthetic construct
RASHGISSWLA

SEQ ID NO: 80 - protein, 15 aa, synthetic construct
WYQQKPGKAPKRLIY

SEQ ID NO: 81 - protein, 7 aa, synthetic construct
AVSTLQT

SEQ ID NO: 82 - protein, 32 aa, synthetic construct
GVPSRFSDSGSGTDLTLTINSLQPEDFATYYC

SEQ ID NO: 83 - protein, 9 aa, synthetic construct
QDLNSYPLS

SEQ ID NO: 84 - protein, 11 aa, synthetic construct
FGGGTKVEIKR

SEQ ID NO: 85 - protein, 118 aa, synthetic construct
EVQLQQSGPELVKPGASVKISCKASGYSFTGYFMHWVKQSHVKSLEWIGRINPYNGDIIYNQNFKDKASLTVDKSSSTAYMELHSLTSEDSAVYYCASLFGLYYFDYWGQGTTLTVSS

SEQ ID NO: 86 - protein, 30 aa, synthetic construct
EVQLQQSGPELVKPGASVKISCKASGYSFT

SEQ ID NO: 87 - protein, 5 aa, synthetic construct
GYFMH

SEQ ID NO: 88 - protein, 14 aa, synthetic construct
WVKQSHVKSLEWIG

SEQ ID NO: 89 - protein, 17 aa, synthetic construct
RINPYNGDIIYNQNFKD

SEQ ID NO: 90 - protein, 32 aa, synthetic construct
KASLTVDKSSSTAYMELHSLTSEDSAVYYCAS

SEQ ID NO: 91 - protein, 9 aa, synthetic construct
LFGLYYFDY

SEQ ID NO: 92 - protein, 118 aa, synthetic construct
QVQLVQSGAEVKKPGATVKISCKASGYSFTGYFMHWVRQAPGKGLEWIGRINPYNGDIIYNQNFKDRATLTVDKSTSTAYMELSSLRSEDTAVYYCASLFGLYYFDYWGQGTTLTVSS

SEQ ID NO: 93 - protein, 30 aa, synthetic construct
QVQLVQSGAEVKKPGATVKISCKASGYSFT

SEQ ID NO: 94 - protein, 14 aa, synthetic construct
WVRQAPGKGLEWIG

SEQ ID NO: 95 - protein, 32 aa, synthetic construct
RATLTVDKSTSTAYMELSSLRSEDTAVYYCAS

SEQ ID NO: 96 - protein, 107 aa, synthetic construct
EIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELKR

SEQ ID NO: 97 - protein, 23 aa, synthetic construct
EIVLTQSPAIMSASPGEKVTMTC

SEQ ID NO: 98 - protein, 10 aa, synthetic construct
SASSSVSYMH

SEQ ID NO: 99 - protein, 15 aa, synthetic construct
WYQQKSGTSPKRWIY

SEQ ID NO: 100 - protein, 7 aa, synthetic construct
DTSKLAS

SEQ ID NO: 101 - protein, 9 aa, synthetic construct
QQWSSNPLT

SEQ ID NO: 102 - protein, 11 aa, synthetic construct
FGAGTKLELKR

SEQ ID NO: 103 - protein, 108 aa, synthetic construct
DVVMTQSPSSVSASVGDRVTITCRASQRISNHLNWYQQKPGKAPKRLIYAASSLESGVPSRFSGSGSGTEFTLTISSLQPEDFATYFCLQHNSYPYTFGQGTKVEIKR

SEQ ID NO: 104 - protein, 23 aa, synthetic construct
DVVMTQSPSSVSASVGDRVTITC

SEQ ID NO: 105 - protein, 11 aa, synthetic construct
RASQRISNHLN

SEQ ID NO: 106 - protein, 7 aa, synthetic construct
AASSLES

SEQ ID NO: 107 - protein, 32 aa, synthetic construct
GVPSRFSGSGSGTEFTLTISSLQPEDFATYFC

SEQ ID NO: 108 - protein, 9 aa, synthetic construct
LQHNSYPYT

SEQ ID NO: 109 - protein, 11 aa, synthetic construct
FGQGTKVEIKR

SEQ ID NO: 110 - protein, 116 aa, synthetic construct
QVQLVQSGAEVAKPGASVKMSCKASGYTFTSYWMHWVRQAPGQGLEWIGYINPSTGYTEYNQKFKDRATLTADKSTSTAYMELSSLRSEDTAVYYCARGYFSFAYWGQGTLVTVSS

SEQ ID NO: 111 - protein, 30 aa, synthetic construct
QVQLVQSGAEVAKPGASVKMSCKASGYTFT

SEQ ID NO: 112 - protein, 32 aa, synthetic construct
RATLTADKSTSTAYMELSSLRSEDTAVYYCAR

SEQ ID NO: 113 - protein, 7 aa, synthetic construct
GYFSFAY

SEQ ID NO: 114 - protein, 11 aa, synthetic construct
WGQGTLVTVSS

SEQ ID NO: 115 - protein, 116 aa, synthetic construct
QVQLVQSGAEVAKPGASVKMSCKASGYTFTSYWMHWVRQAPGQGLEWIGYINPSTGYTEYNQKFKDRATLTADKSKSTAYMELSSLRSEDTAVYYCARGYFSFAYWGQGTLVTVSS

SEQ ID NO: 116 - protein, 23 aa, synthetic construct
DIVMTQSPSSLSASVRDRVTITC

SEQ ID NO: 117 - protein, 11 aa, synthetic construct
QASHDIKNFLN

SEQ ID NO: 118 - protein, 15 aa, synthetic construct
WFQQKPGKAPKLLVY

SEQ ID NO: 119 - protein, 7 aa, synthetic construct
DASNLQV

SEQ ID NO: 120 - protein, 32 aa, synthetic construct
GVPSRFSASGYGTEFTLTISSLQPEDFATYYC

SEQ ID NO: 121 - protein, 9 aa, synthetic construct
QQANSFPRT

SEQ ID NO: 122 - protein, 108 aa, synthetic construct
DIVMTQSPSSLSASVRDRVTITCQASHDIKNFLNWFQQKPGKAPKLLVYDASYLQVGVPSRFSASGYGTEFTLTISSLQPEDFATYYCQQANSFPRTFGQGTKVEIKR

SEQ ID NO: 123 - protein, 7 aa, synthetic construct
DASYLQV

SEQ ID NO: 124 - protein, 108 aa, synthetic construct
DIVMTQSPSSLSASVRDRVTITCQASHDIKNFLNWFQQKPGKAPKLLVYDASHLQVGVPSRFSASGYGTEFTLTISSLQPEDFATYYCQQANSFPRTFGQGTKVEIKR

SEQ ID NO: 125 - protein, 7 aa, synthetic construct
DASHLQV

SEQ ID NO: 126 - protein, 108 aa, synthetic construct
EIVLTQSPSTLSASIGDRVTITCRASEGIYHWLAWYQQKPGKAPKLLIYKASSLASGAPSRFSGSGSGTDFSLTISSLQPEDSATYFCQQAHSFPPTFGGGTKVDIKR

SEQ ID NO: 127 - protein, 23 aa, synthetic construct
EIVLTQSPSTLSASIGDRVTITC

SEQ ID NO: 128 - protein, 11 aa, synthetic construct
RASEGIYHWLA

SEQ ID NO: 129 - protein, 15 aa, synthetic construct
WYQQKPGKAPKLLIY

SEQ ID NO: 130 - protein, 7 aa, synthetic construct
KASSLAS

SEQ ID NO: 131 - protein, 32 aa, synthetic construct
GAPSRFSGSGSGTDFSLTISSLQPEDSATYFC

SEQ ID NO: 132 - protein, 9 aa, synthetic construct
QQAHSFPPT

SEQ ID NO: 133 - protein, 11 aa, synthetic construct
FGGGTKVDIKR

SEQ ID NO: 134 - protein, 120 aa, synthetic construct
QVQLKQSGAELVKPGASVKLSCTASGFNIKDTYMHWVKQRPEQGLEWIARIDPANGNTKYDPKFQGKATMTADTSSNTAYLQLSSLTSEDIAVYYCVRESPYGLYTLDYWGQGTSVTVSS

SEQ ID NO: 135 - protein, 30 aa, synthetic construct
QVQLKQSGAELVKPGASVKLSCTASGFNIK

SEQ ID NO: 136 - protein, 14 aa, synthetic construct
WVKQRPEQGLEWIA

SEQ ID NO: 137 - protein, 32 aa, synthetic construct
KATMTADTSSNTAYLQLSSLTSEDIAVYYCVR

SEQ ID NO: 138 - protein, 11 aa, synthetic construct
ESPYGLYTLDY

SEQ ID NO: 139 - protein, 11 aa, synthetic construct
WGQGTSVTVSS

SEQ ID NO: 140 - protein, 118 aa, synthetic construct
QIQLQQSGAELVKPGASVKLSCKASGYTFTSSWMHWAKQRPGQGLEWIGEIHPNSGNTNYNEKFKGKATLTVDTSSSTAYVDLSSLTSEDSAVYYCARDWDYWYFDVWGAGTSVTVSS

SEQ ID NO: 141 - protein, 30 aa, synthetic construct
QIQLQQSGAELVKPGASVKLSCKASGYTFT

SEQ ID NO: 142 - protein, 5 aa, synthetic construct
SSWMH

SEQ ID NO: 143 - protein, 14 aa, synthetic construct
WAKQRPGQGLEWIG

SEQ ID NO: 144 - protein, 17 aa, synthetic construct
EIHPNSGNTNYNEKFKG

SEQ ID NO: 145 - protein, 32 aa, synthetic construct
KATLTVDTSSSTAYVDLSSLTSEDSAVYYCAR

SEQ ID NO: 146 - protein, 9 aa, synthetic construct
DWDYWYFDV

SEQ ID NO: 147 - protein, 11 aa, synthetic construct
WGAGTSVTVSS

SEQ ID NO: 148 - protein, 117 aa, synthetic construct
QIQLLQSGAELAKPGASVKMSCKASGYTFTSYWMHWVKQRPGQGLEWIGYINPSTGYTEYNQKFKDKATLTADKSSSTAYMQLSSLTSEDSAVYYCARGGYVYFDYWGQGTTLTVSS

SEQ ID NO: 149 - protein, 30 aa, synthetic construct
QIQLLQSGAELAKPGASVKMSCKASGYTFT

SEQ ID NO: 150 - protein, 120 aa, synthetic construct
QVQLKQSGSELVRPGASVKLSCKASGYTFTSYWINWIKQRPGQGLEWIGRIAPGSGINYYNEMFKGKATLTVDTSSSTAYIQLSSLSSEDSAVYFCAGFITKATGAMDYWGQGTSVTVSS

SEQ ID NO: 151 - protein, 30 aa, synthetic construct
QVQLKQSGSELVRPGASVKLSCKASGYTFT

SEQ ID NO: 152 - protein, 5 aa, synthetic construct
SYWIN

SEQ ID NO: 153 - protein, 14 aa, synthetic construct
WIKQRPGQGLEWIG

SEQ ID NO: 154 - protein, 17 aa, synthetic construct
RIAPGSGINYYNEMFKG

SEQ ID NO: 155 - protein, 32 aa, synthetic construct
KATLTVDTSSSTAYIQLSSLSSEDSAVYFCAG

SEQ ID NO: 156 - protein, 11 aa, synthetic construct
FITKATGAMDY

SEQ ID NO: 157 - protein, 121 aa, synthetic construct
EVKLVESGAELVKPGASVKISCKASGYSFTGYYMHWVKQSHGKSLEWIGRVNPNNGGTTYNQKFKGKAILTVDKSSNTAYMELRSLTSEDSAVYYCAKNWGPYGSSYFDYWGQGTTLTVSS

SEQ ID NO: 158 - protein, 30 aa, synthetic construct
EVKLVESGAELVKPGASVKISCKASGYSFT

SEQ ID NO: 159 - protein, 5 aa, synthetic construct
GYYMH

SEQ ID NO: 160 - protein, 17 aa, synthetic construct
RVNPNNGGTTYNQKFKG

SEQ ID NO: 161 - protein, 32 aa, synthetic construct
KAILTVDKSSNTAYMELRSLTSEDSAVYYCAK

SEQ ID NO: 162 - protein, 12 aa, synthetic construct
NWGPYGSSYFDY

SEQ ID NO: 163 - protein, 118 aa, synthetic construct
EVQLQQSGPELVKPGASVKISCKASGYSFTGYFMNWVKQSHGKSLEWIGRINPYNGDTVYNQKFKDKATLTADKSSTTAHMELRSLASEDSAVYFCARWDGNAWFGYWGQGTLVTVSA

SEQ ID NO: 164 - protein, 5 aa, synthetic construct
GYFMN

SEQ ID NO: 165 - protein, 17 aa, synthetic construct
RINPYNGDTVYNQKFKD

SEQ ID NO: 166 - protein, 32 aa, synthetic construct
KATLTADKSSTTAHMELRSLASEDSAVYFCAR

SEQ ID NO: 167 - protein, 9 aa, synthetic construct
WDGNAWFGY

SEQ ID NO: 168 - protein, 11 aa, synthetic construct
WGQGTLVTVSA

SEQ ID NO: 169 - protein, 116 aa, synthetic construct
EVQLQQSGAELAKPGASVKMSCKASGYTFTSYWMHWVKQRPGQGLEWIGYINPSTGYTEYNQKFKDRATLTADKSSSTAYMQLTSLTSEDSAVYYCARGYFSFAYWGQGTLVTVSA

SEQ ID NO: 170 - protein, 30 aa, synthetic construct
EVQLQQSGAELAKPGASVKMSCKASGYTFT

SEQ ID NO: 171 - protein, 32 aa, synthetic construct
RATLTADKSSSTAYMQLTSLTSEDSAVYYCAR

SEQ ID NO: 172 - protein, 124 aa, synthetic construct
EVQLQQSGAELVRPGTSVTLSCKASGYTFSDYEMHWVKQTPVHGLEWIGAIDPETGATAYNQKFKGEATLTADKSSNTAYMELRSLTSEDSAVYYFTRCGYRYDVGEGTSFDYWGQGTSVTVSA

SEQ ID NO: 173 - protein, 30 aa, synthetic construct
EVQLQQSGAELVRPGTSVTLSCKASGYTFS

SEQ ID NO: 174 - protein, 5 aa, synthetic construct
DYEMH

SEQ ID NO: 175 - protein, 14 aa, synthetic construct
WVKQTPVHGLEWIG

SEQ ID NO: 176 - protein, 17 aa, synthetic construct
AIDPETGATAYNQKFKG

SEQ ID NO: 177 - protein, 32 aa, synthetic construct
EATLTADKSSNTAYMELRSLTSEDSAVYYFTR

SEQ ID NO: 178 - protein, 15 aa, synthetic construct
CGYRYDVGEGTSFDY

SEQ ID NO: 179 - protein, 11 aa, synthetic construct
WGQGTSVTVSA

SEQ ID NO: 180 - protein, 107 aa, synthetic construct
DIVMSQSPAIMSASPGEKVTMTCSASSSVSYMYWYQQKPGSSPRLLIYDTSNLASGVPVRFSGSGSGTSYSLTISRMEAEDAATYYCQQWSSYPLTFGGGTKLEIKA

SEQ ID NO: 181 - protein, 23 aa, synthetic construct
DIVMSQSPAIMSASPGEKVTMTC

SEQ ID NO: 182 - protein, 15 aa, synthetic construct
WYQQKPGSSPRLLIY

SEQ ID NO: 183 - protein, 7 aa, synthetic construct
DTSNLAS

SEQ ID NO: 184 - protein, 32 aa, synthetic construct
GVPVRFSGSGSGTSYSLTISRMEAEDAATYYC

SEQ ID NO: 185 - protein, 9 aa, synthetic construct
QQWSSYPLT

SEQ ID NO: 186 - protein, 107 aa, synthetic construct
EIVLTQSPAILSASPGEKVTMTCRARSSVSYIHWYQQKPGSSPKPWIYATSNLASGVPDRFSGSGSGTSYSLTISRVEAEDAATYYCQQWSSTPPTFGAGTKLEIKA

SEQ ID NO: 187 - protein, 23 aa, synthetic construct
EIVLTQSPAILSASPGEKVTMTC

SEQ ID NO: 188 - protein, 10 aa, synthetic construct
RARSSVSYIH

SEQ ID NO: 189 - protein, 7 aa, synthetic construct
ATSNLAS

SEQ ID NO: 190 - protein, 32 aa, synthetic construct
GVPDRFSGSGSGTSYSLTISRVEAEDAATYYC

SEQ ID NO: 191 - protein, 9 aa, synthetic construct
QQWSSTPPT

SEQ ID NO: 192 - protein, 11 aa, synthetic construct
FGAGTKLEIKA

SEQ ID NO: 193 - protein, 112 aa, synthetic construct
DIVMSQSPASLAVSLGQRATISCRASGNVNSYGNSFMHWYQQKPGQPPKLLIYRASNLESGIPARFSGSGSRTDFTLTINPVEADDVATYYCQQSNEDPYTFGGGTKLEIKA

SEQ ID NO: 194 - protein, 23 aa, synthetic construct
DIVMSQSPASLAVSLGQRATISC

SEQ ID NO: 195 - protein, 15 aa, synthetic construct
RASGNVNSYGNSFMH

SEQ ID NO: 196 - protein, 15 aa, synthetic construct
WYQQKPGQPPKLLIY

SEQ ID NO: 197 - protein, 7 aa, synthetic construct
RASNLES

SEQ ID NO: 198 - protein, 32 aa, synthetic construct
GIPARFSGSGSRTDFTLTINPVEADDVATYYC

SEQ ID NO: 199 - protein, 9 aa, synthetic construct
QQSNEDPYT

SEQ ID NO: 200 - protein, 112 aa, synthetic construct
DIVMSQSPTSLAVSLGQRATISCRASESVDSYGNSFMHWYQQKPGQPPKLLIYLASNLESGVPARFSGSGSRTDFTLTIDPVEADDAATYYCQQSNEDPYTFGGGTKLEIKA

SEQ ID NO: 201 - protein, 23 aa, synthetic construct
DIVMSQSPTSLAVSLGQRATISC

SEQ ID NO: 202 - protein, 15 aa, synthetic construct
RASESVDSYGNSFMH

SEQ ID NO: 203 - protein, 7 aa, synthetic construct
LASNLES

SEQ ID NO: 204 - protein, 32 aa, synthetic construct
GVPARFSGSGSRTDFTLTIDPVEADDAATYYC

SEQ ID NO: 205 - protein, 112 aa, synthetic construct
DIVLTQSPASLAVSLGQRATISCRASESVDSYGNSFMHWYQQKPGQPPKLLIYLASNLESGVPARFSGSGSRTDFTLTIDPVEADDAATYYCQQNNEDPRTFGGGTKLEINA

SEQ ID NO: 206 - protein, 23 aa, synthetic construct
DIVLTQSPASLAVSLGQRATISC

SEQ ID NO: 207 - protein, 9 aa, synthetic construct
QQNNEDPRT

SEQ ID NO: 208 - protein, 11 aa, synthetic construct
FGGGTKLEINA

SEQ ID NO: 209 - protein, 109 aa, synthetic construct
DIVLTQSPAIMSASLGERVTMTCTASSSVSSSYLHWYQQKPGSSPKLWIYSTSNLASGVPARFSGSGSGTSYSLTISSVEAEDAATYYCQQYSGYPYTFGGGTKLEIKA

SEQ ID NO: 210 - protein, 23 aa, synthetic construct
DIVLTQSPAIMSASLGERVTMTC

SEQ ID NO: 211 - protein, 12 aa, synthetic construct
TASSSVSSSYLH

SEQ ID NO: 212 - protein, 15 aa, synthetic construct
WYQQKPGSSPKLWIY

SEQ ID NO: 213 - protein, 32 aa, synthetic construct
GVPARFSGSGSGTSYSLTISSVEAEDAATYYC

SEQ ID NO: 214 - protein, 9 aa, synthetic construct
QQYSGYPYT

SEQ ID NO: 215 - protein, 107 aa, synthetic construct
DIMMTQSPAILSASPGEKVTMTCSASSSVSYMYWYQQKPGSSPRLLIYDTSELASGVPARFSGSGSGTSYSLTISSMEAEDAATYYCQQWSSNPYTFGGGTKLEIKA

SEQ ID NO: 216 - protein, 23 aa, synthetic construct
DIMMTQSPAILSASPGEKVTMTC

SEQ ID NO: 217 - protein, 7 aa, synthetic construct
DTSELAS

SEQ ID NO: 218 - protein, 107 aa, synthetic construct
DIVLTQSPAIMSASPGEKVTITCSASSSVSYMHWFQQKPGTSPKLWIYSTSNLASGVPARFSGSGSGTSYSLTISSVEAEDAATYYCHQRSSYPWTFGGGTKLEIKA

SEQ ID NO: 219 - protein, 23 aa, synthetic construct
DIVLTQSPAIMSASPGEKVTITC

SEQ ID NO: 220 - protein, 9 aa, synthetic construct
HQRSSYPWT

SEQ ID NO: 221 - protein, 15 aa, synthetic construct
GGGGSGGGGSGGGGS

SEQ ID NO: 222 - protein, 5 aa, synthetic construct
SSSSG

SEQ ID NO: 223 - protein, 15 aa, synthetic construct
SSSSGSSSSGSSSSG

SEQ ID NO: 224 - protein, 5 aa, synthetic construct
GGGGS

SEQ ID NO: 225 - protein, 113 aa, synthetic construct
QVQLVQSGAEVKKXXASGFNIKDTYMHWVRQAPGQGLEWIGRIDPANGNTKYDPKFQGRATITAGTSINTAYLELSRLRSDDTAVYYCASGPNYYGTTWFAYWGQGTTVTVSS

SEQ ID NO: 226 - protein, 121 aa, synthetic construct
QVQLVQSGXEVNKPGXSVKXSCKASGFNIKDXYMHWVRQAPGQGLEWIGRIDPANGNTKYDPKFQGRATITAYTSFNTAYLELSRLRSDDTAVYYCASGPNYYGTTWFAYWGQGSTVTVSS

SEQ ID NO: 227 - protein, 121 aa, synthetic construct
QVQLVQSGAEVKKPXXSVKLSCKASGFNIKDTYMHWVRQAPGQGLEWIGRIDPENGNTKYDPKFQGRATITAYKSINTAYLELSRLRSDDTAVYYCASGPNYYGTTWFAYWGQGTTVTVSS

SEQ ID NO: 228 - protein, 99 aa, synthetic construct
KASGFNIKDTYMHWVRQAPGQGLEWIGRIDPENGNTKYDPKFQGRATITAYKSINTAYLELSRLRSDDTAVYYCASGPNYYGTTWFAYWGQGTTVTVSS

SEQ ID NO: 229 - protein, 121 aa, synthetic construct
QVQLXQSGAEVKXPGASVKLSCKASGFNIKDTYMHWVRQAPGQGLEWIGRIDPENGNTKYDPKFQGRATITAYKSINTAYLELSRLRSDDTAVYYCASGPNYYGTTWFAYWGQGTTVTVSS

SEQ ID NO: 230 - protein, 121 aa, synthetic construct
QVQLVQSGAEVKKPGASVKLSCKASGFNIKDTYMHWVRQAPGQGLEWIGRIXXENGNTKYDPKFQGRATITAYKSINTAYLELSRLRSDDTAVYYCASGPNYYGTTWFAYWGQGTTVTVSS

SEQ ID NO: 231 - protein, 113 aa, synthetic construct
QVQLVQSGAEVKKPGASGFNIKDTYMHWVRQAPGQGLEWIGRIDPENGNTKYDPKFQGRATITAYKSINTAYLELSRLRSDDTAVYYCASGPNYYGTTWFAYWGQGTTVTVSS

SEQ ID NO: 232 - protein, 111 aa, synthetic construct
EIVLTQSPSTLSASVGDRVTITCRASHGISNYLAWYQQKPGRAPKLLIYKASTLHIGVPSRFSGSGSGTEFALTISSLQPDDFSTYYCQQYNSYLPHTFGQGTRLEIKAAA

SEQ ID NO: 233 - protein, 111 aa, synthetic construct
KIVLTQSPSTLSASVGDRVTITCRASHGISNYLAWYQQKPGRAPKLLIYKASTLHIGVPSRFSGSGSGTEFALTISSLQPDDFSTYYCQQYISYLPHTFGQGTRLEIKAAA

SEQ ID NO: 234 - protein, 26 aa, synthetic construct
VDLKSGGSTSGSGKPGSGEGSSGSAR

SEQ ID NO: 235 - protein, 26 aa, synthetic construct
VDLKSGGSTSDSGKPGSGEGSSGSAR

SEQ ID NO: 236 - protein, 110 aa, synthetic construct
ETTLTQSPSSLSASVGDRITITCRASRAIAKYLAWFQQKPGKAPKSLIYSASTLQSGVPSRFNGSGSGTEFTLTISSLQPEDFATYYCQQTNSFPPTFGQGTRLEIKAAA

SEQ ID NO: 237 - protein, 110 aa, synthetic construct
DIVMTQSPSSLSASIGDRVTITCRASQSISYYLNWYQQKPGKAPKVLIFGASDLQSGVPSRFSGSGSGTEFTLTISSLQPEDFATYYCQQSCRTPRTFGQGTKVEIKAAA

SEQ ID NO: 238 - protein, 110 aa, synthetic construct
EIVLTQSPSSLSASVGDRVTITCRASQTILNFLHWYQQKPGKAPKLLIYDASTLQSGVPSRFSGSASGTDFTLSISNLQPEDFATYYCQQSYSVPRTFGQGTKVEIKAAA

SEQ ID NO: 239 - protein, 110 aa, synthetic construct
EVVMTQSPSSVSASVGGRVTITCRASQGISSYLNWYQQKPGKAPKLLIYAASSLQSGVPSRFSGSGSGTDFTFTISSLQSEDFATYYCQQYYSYPFTFGRGTKVEIKAAA

SEQ ID NO: 240 - protein, 110 aa, synthetic construct
DVVMTQSPSSLSASVGDRVTITCRASQGIRNDLGWYQQKPGKAPKSLIYAASSLQSGVPSRFSGSGSGTGFTLTISSLQPEDFATYYCLQHNSYPFTFGPGTKVEIKAAA

SEQ ID NO: 241 - protein, 110 aa, synthetic construct
DIPMTQSPSSLSASVGDRVTITCQASQGISNYLTWYQQKPGKAPKLLIYAASSLQSGVPSRFSGSGSGTDFTLTISSLQPEDFATYYCQQSNSTPQTFGQGTKVEIKAAA

SEQ ID NO: 242 - protein, 110 aa, synthetic construct
DIQMTQSPSSLSASVGDRVTISCRASQAIRNDLGWYQQKPGQAPRLLIYAASILQSGAPSRFSGSGSETTFTLTIRSLQPDDFATYYCQQSFSVPLTFGGGTKVEIKAAA

SEQ ID NO: 243 - protein, 110 aa, synthetic construct
DIVMTQSPSSLSPSVGDRVTITCRASQTISSYLNWYQQKPGKAPQLLIYAASSLQSGVPSRFSGSGSGTEFTLTISSLQPEDFATCYCQQLNSYPLTFGGGTKVEIKAAA

SEQ ID NO: 244 - protein, 110 aa, synthetic construct
DVVMTQSPSTLSASVGDRVTITCRASQIISSYLAWYQQKPGQAPRLLIYGASSLQSGVPLRFSGSGSGTDFNITISNLQPGDFATYYCQRYNSPPFTFGGGTKVDIKAAA

SEQ ID NO: 245 - protein, 7 aa, synthetic construct
EIKRAAA

SEQ ID NO: 246 - protein, 116 aa, synthetic construct
EVQLQQSGDDLVKPGASVKMXCKASGYTFTSYWMHWVKQRPGQGLEWIGYINPSTGYTEYNQKFKDKATLTADKSSSTAYMQLSSLTSEDSAVYYCARGGNYVDYWGQGTTLTVSS

SEQ ID NO: 247 - protein, 116 aa, synthetic construct
QVQLVQSGAEVKKPGASVKMSCKASGYTFTSYWMHWVRQAPGQGLEWIGYINPSTGYTEYNQKFKDRATLTADKSTSTAYMELRSLRSDDTAVYYCARGGNYVDYWGQGTTLTVSS

SEQ ID NO: 248 - protein, 108 aa, synthetic construct
DVVMTQSPSFLSASVGDRVTVTCRASQSIGSWLAWYQQKPGKAPKLLIYAASSLQSGVPSRFSASGSGTDFTLTISSLQPDDFATYFCHRLNSYPLTFGGGTKVEIKR

SEQ ID NO: 249 - protein, 108 aa, synthetic construct
DIQMTQSPSTLSASVGERVTITCRASQGISSWLAWYQQKPGKAPKRLIYAVSTLQTGVPSRFSGSGSGTDLTLTINSLQPEDFATYYCQHLNSYPLTFGGGTKVEIKR

SEQ ID NO: 250 - protein, 107 aa, synthetic construct
DIQMTQSPSTLSASVGERVTITCRASQGISSWLAWYQQKPGKAPKRLIYAVSTLQTGVPSRFSGSGSGTDLTLTINSLQPEDFATYYCQHLNSYPLTFGGGTKVEIK

SEQ ID NO: 251 - protein, 110 aa, synthetic construct
DIQMTQSPSTLSASVGERVTITCRASQGISSWLAWYQQKPGKAPKRLIYAVSTLQTGVPSRFSGSGSGTDLTLTINSLQPEDFATYYCQHLNSYPLTFGGGTKVEIKAAA

SEQ ID NO: 252 - protein, 110 aa, synthetic construct
DIQMTQSPSTLSASVGERVTITCRASQDISSWLAWYQQKPGKAPKRLIYAVSTLQTGVPSRFSGSGSGTDLTLTINSLQPEDFATYYCQDLNSYPLTFGGGTKVEVKAAA

SEQ ID NO: 253 - protein, 110 aa, synthetic construct
DIQMTQSPSTLSASVGERVTITCRASHGISSWLAWYQQKPGKAPKRLIYAVSTLQTGVPSRFSDSGSGTDLTLTINSLQPEDFATYYCQDLNSYPLSFGGGTKVEIKAAA

SEQ ID NO: 254 - protein, 116 aa, synthetic construct
DIVMTQSPDSLAVSLGERATINCKSSQSVLYSSNNKNYLAWYQQKPGQPPKLLIYWASTRESGVPDRFSGSGSGTDFTLTISILQAEDVAVYYCQQYYSTPYTFGQGTKVDIKAAA

SEQ ID NO: 255 - protein, 111 aa, synthetic construct
DVVMTQSPSTLSASVGDRVTITCRASENINNYLSWYQQKPGKAPKFLINAASSLQSGVPSRFSGSGSGTDFTLTISSLQPEDFASYYCQQVNIFPPKTFGQGTKVEIKAAA

SEQ ID NO: 256 - protein, 110 aa, synthetic construct
DVVMTQSPSSLSASVGDRVTITCRASQGTSSWLAWYQQKPEKAPKSLIYATSSLQSGVPSRFSGSGSGTDFTLTISYLQSDDFATYYCQQYLEYPVTFGGGTKVEIKAAA

SEQ ID NO: 257 - protein, 109 aa, synthetic construct
XXVMTQSPSSLSASVGDRVTITCQASQDISNYLNWYQQKPGKAPKLLIYDASNLQTGVPSRFXGSGSGTDFTLTISSLQPEDFATYYCQQHXSFPLTFGGGTKVEIKAA

SEQ ID NO: 258 - protein, 110 aa, synthetic construct
EIVLTQSPSSVSASVGDRVTITCRASQGISXWLAWYQQKPGKAPKFLIYDASSLQSGVPSKFSGSGSGTDFTLTISSLQPEDFATYYCQKYNSAPFTFGQGTKVEIKAAA

SEQ ID NO: 259 - protein, 110 aa, synthetic construct
EXVMTQSPSSLSASVGDRVTITCRASQGIRNHLGWYQQKPGKAPKRLIYAASSLQSGVPSRFSGSGSGTEFTLTISSLQPEDFATYYLQHNYYTPLTFGGGTKVEIKAAA

SEQ ID NO: 260 - protein, 110 aa, synthetic construct
DIVMTQSPSSLSASVGDRVTITCRASQGVRNHLGWFQQKPGKAPKSLIYAASSLQSGVPSKFSGSGSGTEFTLTISRLQPEDFATYYCLQHNSYPLTFGGGTKVEIKAAA

SEQ ID NO: 261 - protein, 110 aa, synthetic construct
DVVMTQSPSSLSASVGDRVTITCRASQSVSSYLNWYQQKPGKAPKLLIYAASSLQNGVPSRFSGSGSGTDFTLTISSLQPEDFATYYCQQSYSTPLTFGGGTKVEIKAAA

SEQ ID NO: 262 - protein, 111 aa, synthetic construct
EIVLTQSPSSLSAFVGDRVTISCRASQSIRHYVTWYQQRPGKAPELLISGASRLQSGVPSWFSGSGSGTDFTLTISSLQPEDFATYYCQQSYSTPLWTFGQGTKVEIKAAA

SEQ ID NO: 263 - protein, 111 aa, synthetic construct
DVVMTQSPSTLSASVGDRVTITCRASQGISSWLAWYQQKPGKAPNLLIYAASSLQSGVPDRFSGSGXGTEFTLTISGLQPEDFATYYCQQLNSYPMYTFGQGTKVEIKAAA

SEQ ID NO: 264 - protein, 110 aa, synthetic construct
DVVMTQSPSSVSASVGDRVTITCRASQGISSWLAWYQQKPGKAPKLLIYATSSLQSGVPSRFSGSGSGTEFTLTISSLQPEDFATYYCLHHNSYPLTFGQGTKVEIKAAA

SEQ ID NO: 265 - protein, 126 aa, synthetic construct
EVQLQQSGPELVKPGASVKISCKASGYSFTGYFMHWVKQSHVKSLEWIGRINPYNGYDIIYNQNFKDKASLTVDKSSSTAYMELHSLTSEDSAVYYCASAVYYCASLFGLYYFDYWGQGTTLTVSS

SEQ ID NO: 266 - protein, 126 aa, synthetic construct
EVQLVQSGAEVKKPGASTKISCKASGYSFTGYFMHWVRQAPGKGLEWIGRINPYNGYDIIYNQNFKDRATLTVDKSSSTAYMELSSLRSEDSAVYYCATAVYYCASLFGLYYFDYWGQGTTLTVSS

SEQ ID NO: 267 - protein, 109 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELKAAA

SEQ ID NO: 268 - protein, 110 aa, synthetic construct
EIVLTQSPSSLSASVGDRVTITCRASQSISSYLNWYQQKPGKAPKRLIYAASSLQSGVPSRFSGSGSGTEFTLTISSLQPEDFATYYCLQHNTYPWTFGQGTKVEIKAAA

SEQ ID NO: 269 - protein, 110 aa, synthetic construct
DVVMTQSPSSVSASVGDRVTITCRASQRISNHLNWYQQKPGKAPKRLIYAASSLESGVPSRFSGSGSGTEFTLTISSLQPEDFATYFCLQHNSYPYTFGQGTKVEIKAAA

SEQ ID NO: 270 - protein, 110 aa, synthetic construct
DIVQMQSPSSLSASVGDRVTITCRASQSISSYLNWYQQKPGKAPKRLIYAASSLQSGVPSRFSGSGSGTEFTLTISSLQPEDFATYYCLQHNSYPWTFGQGTKVEIKAAA

SEQ ID NO: 271 - protein, 110 aa, synthetic construct
DIVQMQSPSSVSASVGDRVTITCRASQSIYSYLNWYQQKPGKAPKRLIYAASSLQSGVPSRFSGSGSGTEFTLTISSLQPEDFATYYCLQHNSYPWTFGQGTKLEIKAAA

SEQ ID NO: 272 - protein, 119 aa, synthetic construct
QVQLVQSGAEVKKPGASVKVSCKASDYSFTDYNIYWVRQAPGQGLEWIGYIDPYNGGTSYNQKFKGRATLTVDKSISTAYMELSRLRSDDTAVYYCALYGYDPDYIDYWGQGTTLTVSS

SEQ ID NO: 273 - protein, 119 aa, synthetic construct
QVQPVQSGAEVKKPGASVKVSCKASDYSFTDYNIYWVRQAPGQGLEWIGYIDPYNGGTSYNQKYRGRATLTVDKSINTAYMELSRLRSDDTAVYYCALYGYDPDYIEYWGQGTTLTVSS

SEQ ID NO: 274 - protein, 119 aa, synthetic construct
QVQLVQSGAEVKKPGASVKVSCKASDYSFTDYNIYWVRQAPGQGLEWIGYIDPYNGGTSYNQKFKGRATLTVDKSISTAYMELSRLRSDDTAVYYCALYGYDPDYIDYWGPGTTLTVSS

SEQ ID NO: 275 - protein, 106 aa, synthetic construct
DIVLTQSPASMAASPGQRVTITCSASSSVSYLHWFQQKPGQSPKLWIYSTSNLASGVPARFSGSGSGTDYTLTINPMEANDTATYYCQQRSSYPYTFGQGTKLEIK

SEQ ID NO: 276 - protein, 109 aa, synthetic construct
DIVLTQSPASMAVSPGQRVTITCSASSSVSYLHWFQQKPGQSPKLWIYSTSNLASGVPARFSGSGSGTDYTLTINPMEAEDTATYYCQQRSSYPYTFGQGTKLEIKAAA

SEQ ID NO: 277 - protein, 109 aa, synthetic construct
DIVLTQSPASMAASPGQRVTITCSASSSVSYLHWFQQKPGQSPKLWIYSTSNLASGVPARFSGSGSGTDYTLTINPMEAEDTATYYCQQRSSYPYTFGQGTKLEIKAAA

SEQ ID NO: 278 - protein, 109 aa, synthetic construct
DIVLTQSPASMAASPGQRVTITCSASSSVSYLHWFQQKPGQSPKLMIYGTSNLASGVPVRFSGSGSGTSYSLTINPMEAEDTATYYCQQRSSYPYTFGQGTKLEIKAAA